(19) United States
(12) Patent Application Publication
(10) Pub. No.: US 2025/0115596 A1
(43) Pub. Date: Apr. 10, 2025

(54) ESTERS OF 8-METHYL-8-AZABICYCLO[3.2.1] OCTAN-3-YL 3-HYDROXY-2-PHENYLPROPANOATE

(71) Applicant: ADS Therapeutics LLC, Irvine, CA (US)

(72) Inventors: Jinsong Ni, Irvine, CA (US); Rong Yang, Irvine, CA (US); Wenkui Fang, Irvine, CA (US)

(21) Appl. No.: 18/729,941

(22) PCT Filed: Jan. 19, 2023
(86) PCT No.: PCT/US2023/011146

§ 371 Date: Jul. 18, 2024

Related U.S. Application Data
(60) Provisional application No. 63/301,657, filed on Jan. 21, 2022.

(51) Int. Cl. C07D 451/10; A61K 31/46; A61P 27/02

(52) U.S. Cl. CPC C07D 451/10; A61K 31/46; A61P 27/02

(57) ABSTRACT

In one general aspect, the present disclosure provides fatty acid esters of atropine, or a pharmaceutically acceptable salt thereof. A pharmaceutical composition comprising these esters is also provided. In one example, the composition comprises a semifluorinated alkane vehicle, such as perfluorohexyloctane (F6H8). Method of using the fatty esters of atropine include medical fields where atropine is known to be useful. In one example, the disclosure provides methods of using the compound as cycloplegic agent and/or as mydriatic.

### CLAIM OF PRIORITY

This application claims priority to U.S. Provisional Patent Application Ser. No. 63/301,657, filed on Jan. 21, 2022, the entire contents of which are hereby incorporated by reference.

### TECHNICAL FIELD

This disclosure relates to esters, and in particular to fatty acid esters, of 8-methyl-8-azabicyclo[3.2.1]octan-3-yl 3-hydroxy-2-phenylpropanoate (e.g., atropine), processes for preparing them, pharmaceutical compositions containing them, and their use as pharmaceutical agents to treat and prevent diseases in which muscarinic acetylcholine receptors are implicated.

### BACKGROUND

Muscarinic acetylcholine receptors, or mAChRs, are acetylcholine receptors that form G protein-coupled receptor complexes in the cell membranes of certain neurons, such as ganglia. There are five subtypes of muscarinic acetylcholine receptors, named M₁-M₅. M₁, M₃, M₅ receptors are coupled with G_{q} proteins, while M₂ and M₄ receptors are coupled with G_{i/o} proteins. M₁ receptors are typically implicated in excitatory post-synaptic potential, secretion from salivary glands and stomach, higher cognitive processes such as learning and spatial memory, and in analgesia. M₂ receptors are typically implicated in slowing heart rate, reducing contractile forces of atrium, reducing conduction velocity of atrioventricular node, and in homotropic inhibition. M₃ receptors are typically implicated in smooth muscle contraction, bronchoconstriction and increased lung secretion, increase in intracellular calcium in vascular endothelium, increased endocrine and exocrine gland secretions (e.g., salivary glands and stomach), increased gastrointestinal motility, eye accommodation (miosis when M₃ is stimulated or mydriasis when M₃ is inhibited), iris sphincter muscle functioning, cerebral vascular vasodilation, systemic vascular vasodilation (depending on the location of M₃), and in inducing emesis. M₄ receptors are typically implicated in locomotion (e.g., activation of the receptor causes decreased locomotion). M₅ receptors are implicated in numerous CNS functions. Atropine, for example, is a competitive antagonist of the muscarinic acetylcholine receptor types M₁, M₂, M₃, M₄ and M₅, and as such, may modulate any of the aforementioned physiological processes.

### SUMMARY

The instant disclosure is based, at least in part, on a realization that fatty acid esters, including unsaturated fatty acid esters, of (1R,3r,5S)-8-methyl-8-azabicyclo[3.2.1]octan-3-yl 3-hydroxy-2-phenylpropanoate (also known as atropine) are useful as prodrugs. Upon hydrolytic and/or enzymatic cleavage of the ester functionality, the parent compound (e.g., atropine) is released to act as a competitive antagonist of the muscarinic acetylcholine receptor types M₁, M₂, M₃, M₄ and M₅. What is more, and importantly here, the fatty acid (e.g., saturated or unsaturated) ester prodrugs of the parent compound (e.g., atropine) show superior solubility to atropine in fluorinated solvents, such as a semifluorinated alkane solvent F6H8 (perfluorohexyloctane). F6H8 is a pharmaceutically acceptable amphiphilic liquid with two mutually immiscible moieties (hydrocarbon segment RH and perfluorinated segment RF) that are bound covalently. Hence, these fluorinated solvents are advantageously useful as liquid vehicles for the delivery of the ester prodrugs of atropine to patients in need thereof. In one aspect, it was surprisingly found that fatty acid esters (e.g., unsaturated and/or saturated fatty acid esters) of atropine are about 10×, about 100×, or about 1,000× more soluble in semifluorinated alkane solvents such as F6H8, compared to the atropine. In one example, it was surprisingly found that saturated fatty acid esters of atropine are about 10×, about 100×, or about 1,000× more soluble in semifluorinated alkane solvents such as F6H8, compared to the atropine. In another example, it was surprisingly found that fatty acid esters of atropine are about 10×, about 100×, about 1,000×, or about 10,000× more soluble in semifluorinated alkane solvents such as F6H8, compared to atropine.

In one general aspect, the present disclosure provides a compound of Formula (I):

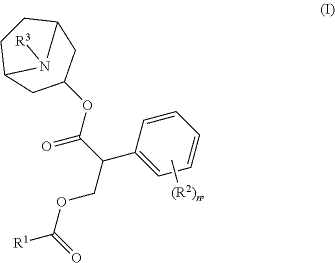

or a pharmaceutically acceptable salt thereof, wherein R¹, R², n, and R³ are as described herein.

In another general aspect, the present disclosure provides a pharmaceutical composition comprising a compound of Formula (I), or a pharmaceutically acceptable salt thereof, and a pharmaceutically acceptable carrier.

In some embodiments, the pharmaceutically acceptable carrier comprises a semifluorinated alkane. In some embodiments, the semifluorinated alkane is selected from perfluorohexyloctane (F6H8), perfluorohexylhexane (F6H6), perfluorobutylbutane (F4H4), and perfluorohexylethane (F6H2). In some embodiments, the pharmaceutical composition is an ophthalmic composition. Suitable examples of ophthalmic compositions include eye drops comprising an emulsion, a suspension, or a solution of the compound of Formula (I), or a pharmaceutically acceptable salt thereof.

In another general aspect, the present disclosure provides a method of treating or preventing a disease or condition where muscarinic acetylcholine receptor is implicated, the method comprising administering to a subject in need thereof a therapeutically effective amount of a compound of Formula (I), or a pharmaceutically acceptable salt thereof, or a pharmaceutical composition comprising same.

In some embodiments, the compound of Formula (I), or a pharmaceutically acceptable salt thereof, or a pharmaceutical composition comprising same are used for causing cycloplegic refraction in the eye of the subject, for causing mydriasis in the eye of the subject, for treating amblyopia or lazy eye in children, for relieving vitreous floater symptoms, for treating or preventing painful ciliary muscle spasm or for treating myopia progression in pediatric subjects.

In some embodiments, the compound of Formula (I), or a pharmaceutically acceptable salt thereof, or a pharmaceutical composition comprising same are used for treating or preventing a heart condition, such as a low heart rate (bradycardia), for reducing salivation and bronchial secretions (e.g., before surgery), or for treating or preventing is hyperhidrosis or a poisoning. In these embodiments, the compound of Formula (I), or a pharmaceutically acceptable salt thereof, or a pharmaceutical composition comprising same, are administered to the subject by oral, intramuscular, intravenous, subcutaneous, intraosseous, or endotracheal route, or by inhalation.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### BRIEF DESCRIPTION OF DRAWINGS

FIG. 1 is a line plot showing pupil diameter change in rabbits following ophthalmic administration of vehicle, atropine oleate, atropine linoleate, and atropine sulfate.

### DETAILED DESCRIPTION

In some embodiments, the present disclosure provides, inter alia, fatty acid ester prodrugs of tropane alkaloids such as atropine. Without being bound by any theory or speculation, it is believed and understood that upon administration of the ester to a patient and subsequent hydrolytic and/or enzymatic cleavage, the parent alkaloid is released from its prodrug form to act as a competitive antagonist of the muscarinic acetylcholine receptor types M₁, M₂, M₃, M₄ and M₅. As such, the compounds of the present disclosure are useful in treating or preventing diseases, disorders, and conditions where the acetylcholine receptors are implicated.

In some embodiments, the compounds of the instant disclosure are useful in treating uveitis and/or for causing cycloplegic refraction and/or mydriasis (dilation of pupil) in the eye of the patient. When used as cycloplegic agents, the compounds within the instant claims temporarily paralyze ciliary muscle of the eye and concomitantly paralyze the accommodation reflex. This is useful, for example, during eye examination, to obtain accurate refraction and the true refractive error of the eye, e.g., in young children. The compounds can useful for treating amblyopia or lazy eyes in children. The compounds are useful for relieving vitreous floater symptoms. The compounds can also alleviate a painful ciliary muscle spasm in the eye. When used as a mydriatic, for the dilation of the pupil, the instant compounds allow for examination of the retina and other deep structures of the eye. As mydriatics, the compounds are also useful to prevent myopia progression in children. When used to treat or prevent a condition in the eye of the patient, the compound may be administered and/or applied as any one of the topical/ophthalmic compositions as described herein (e.g., as an eye drop of an emulsion, a suspension, or a solution).

In some embodiments, the compounds of the instant disclosure are useful in any medical field where atropine, scopolamine, or hyoscyamine are advantageously used to treat or prevent a condition. In some embodiments, the compounds are useful for treating or preventing various heart conditions. For example, the compounds can treat or prevent the symptoms of low heart rate (bradycardia). In some embodiments, the compounds are useful to treat bradycardia when the heart rate of the patient is less than about 60 beats per minute. For this application, the compound can be administered by an IV injection. For symptomatic bradycardia, the usual dosage of atropine is from about 0.5 mg to about 1 mg IV push, which may be repeated every 3 to 5 minutes up to a total dose of about 3 mg (maximum dose of about 0.04 mg/kg). The dosage of the compound of this disclosure may be adjusted accordingly based on the difference in molecular weight of atropine and the ester compound of this disclosure. The compounds of this disclosure are also useful in treating second-degree heart block Mobitz Type 1, and also third degree heart block with a high purkinje or AV-nodal escape rhythm. In some embodiments, the compounds can be used to prevent a low heart rate during intubation of children.

In some embodiments, the compounds of the instant disclosure are useful to reduce salivation and bronchial secretions (e.g., before a surgery). Atropine's actions on the parasympathetic nervous system inhibit salivary and mucus glands. Hence, the compound of Formula (I) may inhibit sweating via the sympathetic nervous system. This can be useful in treating hyperhidrosis, and can prevent the death rattle of dying patients.

In some embodiments, the compounds of the instant disclosure are useful as antidote to poisoning. For example, atropine serves as a treatment for poisoning by organophosphate insecticides and nerve agents, such as tabun, sarin, soman, and VX. In a developed case of nerve-gas poisoning, maximum atropinization is desirable. The compound of Formula (I) can be used in conjunction with the oxime pralidoxime chloride, or any other of the anti-poisoning dots provided herein. For the systemic indications, the compounds can be administered by any of the routes described herein, for example, by oral, intramuscular, intravenous, subcutaneous, intraosseous, or endotracheal route, or by inhalation.

### Therapeutic Compounds

In one general aspect, the present disclosure provides a compound of Formula (I):

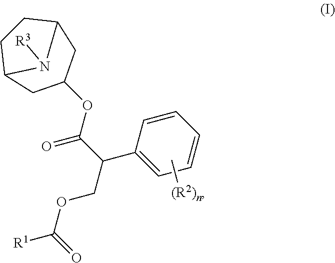

or a pharmaceutically acceptable salt thereof, wherein:
R³ is selected from H, C₁₋₆ alkyl, and C₁₋₆ haloalkyl;
each R² is independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino;
n is selected from 0, 1, 2, 3, 4, and 5; and
R¹ is selected from C₇₋₃₄ alkyl, C₇₋₃₄ alkenyl, C₇₋₃₄ alkynyl, C₃₋₁₀ cycloalkyl, 4-10 membered heterocycloalkyl, 5-14 membered heteroaryl, and C₆₋₁₀ aryl, wherein said C₇₋₃₄ alkyl, C₇₋₃₄ alkenyl, and C₇₋₃₄ alkynyl are each optionally substituted with 1 or 2 substituents independently selected from C₃₋₁₀ cycloalkyl, 4-10 membered heterocycloalkyl, 5-14 membered heteroaryl, and C₆₋₁₀ aryl, and each of said C₃₋₁₀ cycloalkyl, 4-10 membered heterocycloalkyl, 5-14 membered heteroaryl, and C₆₋₁₀ aryl is optionally substituted with 1, 2, or 3 substituents independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino.

In some embodiments, the compound of Formula (I) is not atropine oleate or hyoscyamine oleate. In some embodiments, the compound of Formula (I) is not atropine oleate. In some embodiments, the compound of Formula (I) is not hyoscyamine oleate.

In some embodiments, R¹ is selected from C₇₋₃₄ alkyl, C₇₋₃₄ alkenyl, and C₇₋₃₄ alkynyl.

In some embodiments, R¹ is selected from C₇₋₂₁ alkyl, C₇₋₂₁ alkenyl, and C₇₋₂₁ alkynyl. In some embodiments, R¹ is selected from C₇₋₂₁ alkyl, C₂₋₂₁ alkenyl (e.g., comprising only one double bond), and C₂₋₂₁ alkynyl.

In some embodiments, R¹ is selected from C₃₋₁₀ cycloalkyl, 4-10 membered heterocycloalkyl, 5-14 membered heteroaryl, and C₆₋₁₀ aryl, each of which is optionally substituted with 1, 2, or 3 substituents independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino. In some embodiments, R¹ is C₃₋₁₀ cycloalkyl. In some embodiments, R¹ is 4-10 membered heterocycloalkyl. In some embodiments, R¹ is 5-14 membered heteroaryl. In some embodiments, R¹ is C₆₋₁₀ aryl.

In some embodiments, R¹ is selected from C₇₋₃₄ alkyl, C₇₋₃₄ alkenyl, and C₇₋₃₄ alkynyl, each of which is optionally substituted with 1 or 2 substituents independently selected from C₃₋₁₀ cycloalkyl, 4-10 membered heterocycloalkyl, 5-14 membered heteroaryl, and C₆₋₁₀ aryl (and each of these cyclic groups is optionally substituted as described above).

In some embodiments, R¹ is C₁₋₆ alkyl, optionally substituted with C₃₋₁₀ cycloalkyl, 4-10 membered heterocycloalkyl, 5-14 membered heteroaryl, or C₆₋₁₀ aryl, each of which is optionally substituted with 1, 2, or 3 substituents independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino. In some embodiments, R¹ is C₁₋₆ alkyl optionally substituted with C₃₋₁₀ cycloalkyl. In some embodiments, R¹ is C₁₋₆ alkyl optionally substituted with 4-10 membered heterocycloalkyl. In some embodiments, R¹ is C₁₋₆ alkyl optionally substituted with 5-14 membered heteroaryl. In some embodiments, R¹ is C₁₋₆ alkyl optionally substituted with C₆₋₁₀ aryl.

In some embodiments, R¹ is C₁₋₃₄ alkyl. In some embodiments, R¹ is C₇₋₃₄ alkyl. In some embodiments, R¹ is C₇₋₂₁ alkyl.

In some embodiments, R¹ is C₁₋₆ alkyl. For example, R¹ is selected from methyl, ethyl, propyl, isopropyl, n-butyl, sec-butyl, tert-butyl, iso-butyl, n-pentyl, neo-pentyl, or n-hexyl. In some embodiments, the compounds of Formula (I) where R¹ is C₁₋₆ alkyl are administered to a patient to treat or prevent an ophthalmic condition and/or in a pharmaceutical formulation with a semifluorinated alkane liquid vehicle as described herein.

In some embodiments, R¹ is C₇₋₁₁ alkyl. For example, R¹ is a residue of a saturated medium-chain fatty acid, such as caproic acid (hexanoic acid), caprylic acid (octanoic acid), pelargonic acid (nonanoic acid), capric acid (decanoic acid), or undelyc acid (undecanoic acid).

In some embodiments, R¹ is C₁₂₋₂₁ alkyl. For example, R¹ is a residue of a saturated long-chain fatty acid, such as lauric acid (dodecanoic acid), tridecylic acid (tridecanoic acid), myristic acid (tetradecanoic acid), pentadecylic acid (pentadecanoic acid), palmitic acid (hexadecanoic acid), margaric acid (heptadecanoic acid), stearic acid (octadecanoic acid), nonadecylic acid (nonadecanoic acid), arachidic acid (eicosanoic acid), or heneicosylic acid (heneicosanoic acid).

In some embodiments, R¹ is C₂₂₋₃₄ alkyl. For example, R¹ is a residue of a saturated very long-chain fatty acid, such as lignoceric acid (tetracosanoic acid), cerotic acid (hexacosanoic acid), montanic acid (octacosanoic acid), lacceroic acid (dotriacontanoic acid), or geddic acid (tetratriacontanoic acid).

In some embodiments, R¹ is C₂₋₃₄ alkenyl. In some embodiments, R¹ is C₇₋₃₄ alkenyl. In some embodiments, R¹ is C₇₋₂₁ alkenyl. In some embodiments, any of the alkenyl chains of R¹ comprises one, two, three, four, five, or six double bonds.

In some embodiments, R¹ is C₂₋₆ alkenyl. In some embodiments, R¹ is C₂₋₆ alkenyl comprising one double bond. In some embodiments, R¹ is C₄₋₆ alkenyl comprising two double bonds. For example, R¹ is selected from vinyl, propenyl, butenyl, or hexadienyl.

In some embodiments, R¹ is C₇₋₁₁ alkenyl. In some embodiments, R¹ is C₇₋₁₁ alkenyl comprising one double bond. In some embodiments, R¹ is C₇₋₁₁ alkenyl comprising two double bonds. For example, R¹ is a residue of an unsaturated medium-chain fatty acid, such as 5-caproleic acid (dec-5-enoic acid), 4-caproleic (dec-4-enoic) acid, or 9-caproleic (dec-9-enoic) acid.

In some embodiments, R¹ is C₁₂₋₂₁ alkenyl. In some embodiments, R¹ is C₁₂₋₂₁ alkenyl comprising one double bond. In some embodiments, R¹ is C₁₂₋₂₁ alkenyl comprising two double bonds. For example, R¹ is a residue of an unsaturated long-chain fatty acid, such as lauroleic (9-dodecenoic) acid, sapienic (hexadec-6-enoic) acid, palmitoleic (hexadec-9-enoic) acid, myristoleic (tetradec-9-enoic) acid, oleic (Z-octadec-9-enoic) acid, and linoleic (octadeca-9,12-dienoic) acid, palmitoleic (hexadec-9-enoic) acid, elaidic (E-octadec-9-enoic) acid, vaccenic (octadec-11-enoic) acid, linoelaidic acid, linolenic (octadeca-9,12-dienoic) acid, vaccenic (octadec-11-enoic) acid, petroselinic (octadec-6-enoic acid) acid, oct-3-enoic acid, or arachidonic (icosa-5,8,11,14-tetraenoic) acid. In some embodiments, any of double bonds within the alkeynyl or alkynyl of R¹ is in E or Z configuration.

In some embodiments, R¹ is C₂₂₋₃₄ alkenyl. For example, R¹ is a residue of a unsaturated very long-chain fatty acid, such as nervonic acid (Z-tetracos-15-enoic acid), ximenic acid (Z-17-hexacosenoic acid), or lumequeic acid (Z-21-triacontenoic acid).

In some embodiments, R¹ is C₂₋₃₄ alkynyl. In some embodiments, R¹ is C₇₋₃₄ alkynyl.

In some embodiments, R¹ is C₇₋₂₁ alkynyl. In some embodiments, the any of the alkynyl chains of R¹ comprises one, two, or three triple bonds. In one example, the alkynyl chain comprises one triple bond and one double bond. In another example, the alkynyl chain comprises one triple bond and two double bonds.

In some embodiments, n is selected from 0, 1, 2, and 3. In some embodiments, n is selected from 1, 2, and 3. In some embodiments, n is 0.

In some embodiments, R² is selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, and di(C₁₋₃ alkyl)amino.

In some embodiments, R² is selected from C₁₋₃ alkyl, C₁₋₃ haloalkyl, C₁₋₃ alkoxy, C₁₋₃ haloalkoxy, OH, NO₂, CN, halo, and amino.

In some embodiments, R³ is H.

In some embodiments, R³ is C₁₋₆ alkyl (e.g., methyl, ethyl, propyl, or isopropyl).

In some embodiments, R³ is C₁₋₆ haloalkyl (e.g., trifluoromethyl, 2,2,2-trifluoroethyl, or 2-fluoroethyl).

In some embodiments, n is 0 and R³ is C₁₋₃ alkyl.

In some embodiments, the compound of Formula (I) has formula:

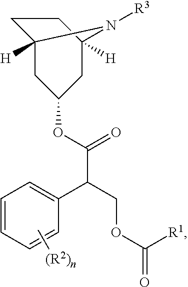

or a pharmaceutically acceptable salt thereof.

In some embodiments, the compound of Formula (I) has formula:

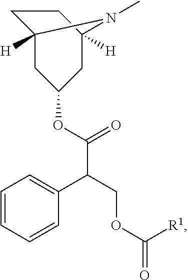

or a pharmaceutically acceptable salt thereof.

In some embodiments, the compound of Formula (I) has formula:

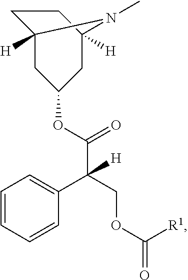

or a pharmaceutically acceptable salt thereof.

In some embodiments, the compound of Formula (I) has formula:

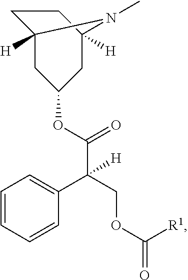

or a pharmaceutically acceptable salt thereof.

In some embodiments, the compound of Formula (I) is selected from atropine 5-caproleate, atropine 4-caproleate, atropine 5-lauroleate, atropine sapienate, atropine palmitoleate, atropine myristoleate, atropine oleate, atropine vaccenate, atropine petroselinate, atropine 3-octenoate, atropine linoleate, atropine caprate, atropine caprylate, atropine laurate, atropine myristate, and atropine palmitate, or a pharmaceutically acceptable salt thereof.

In some embodiments, the compound of Formula (I) is selected from any one of the following compounds:

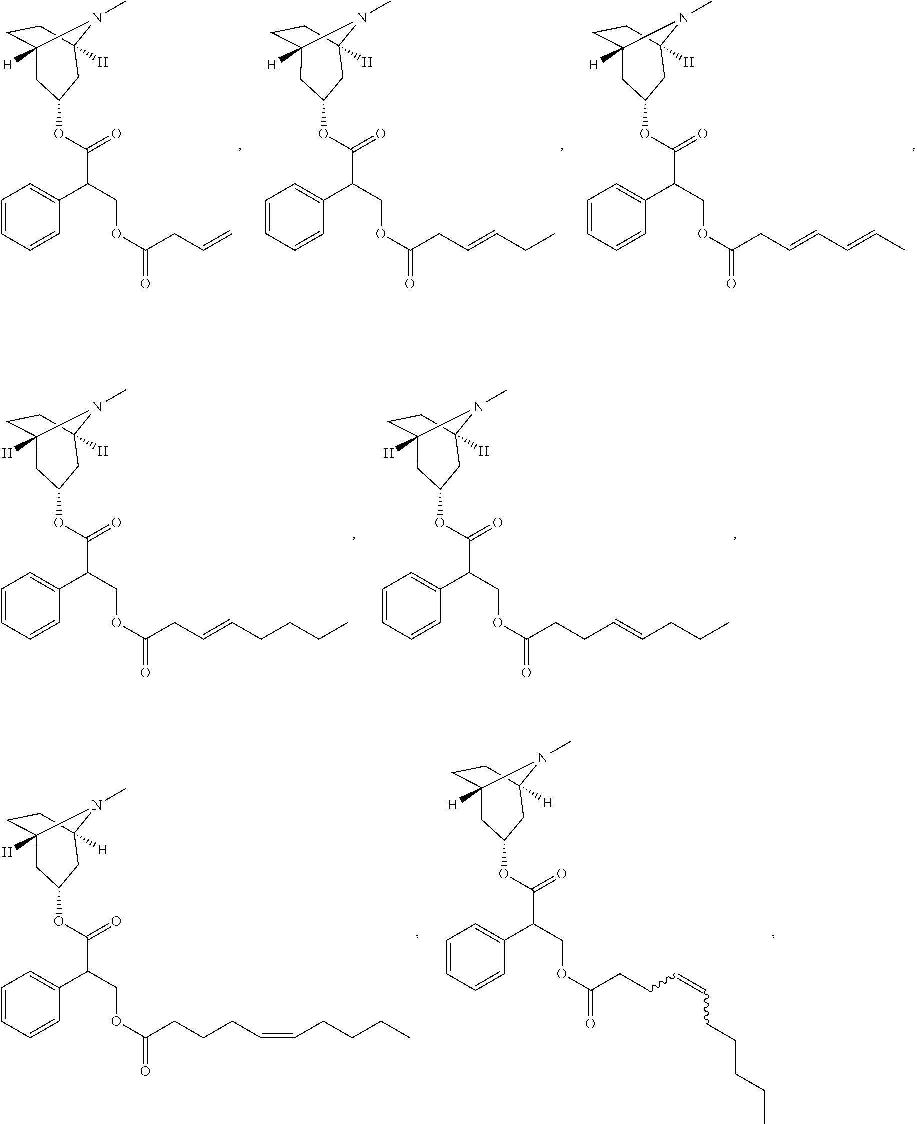

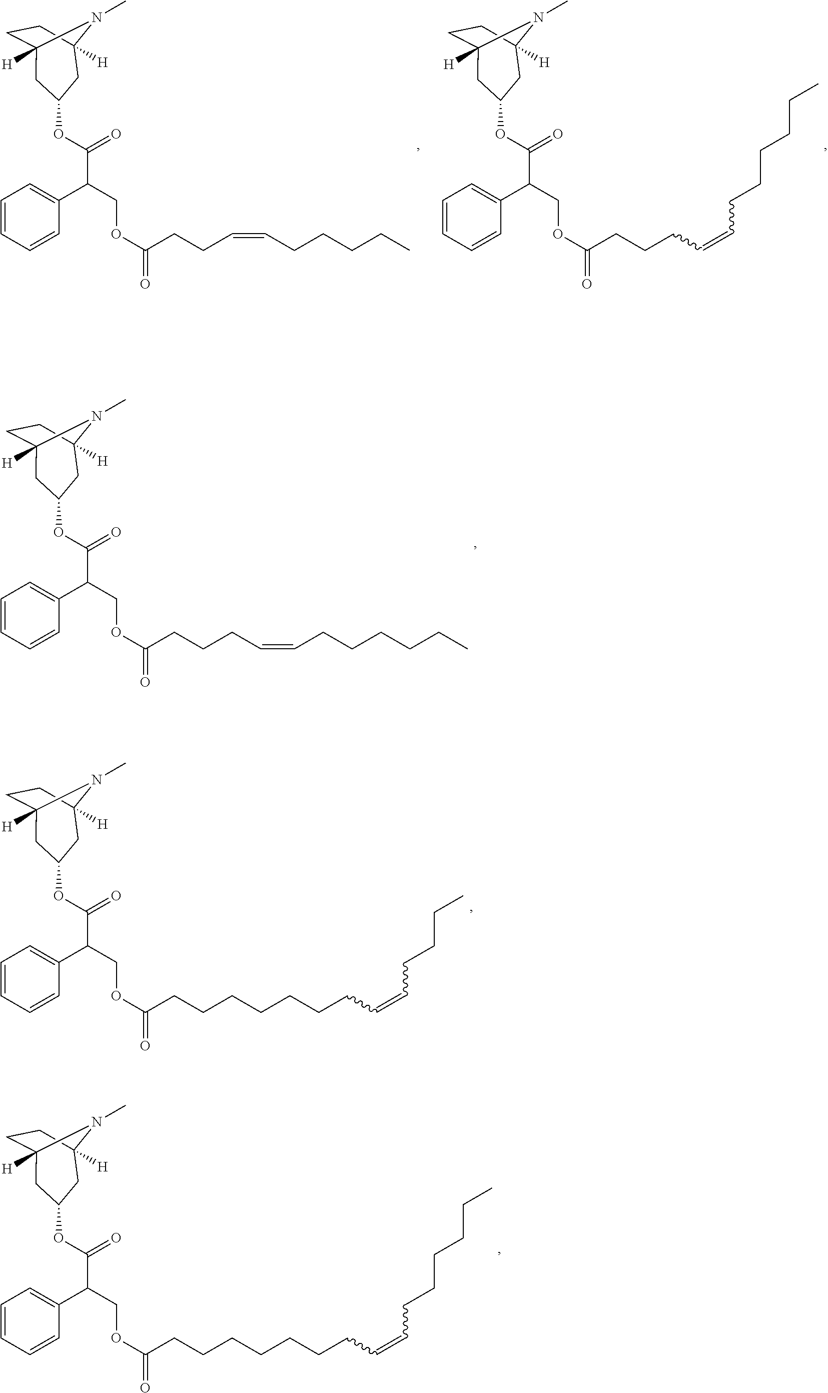

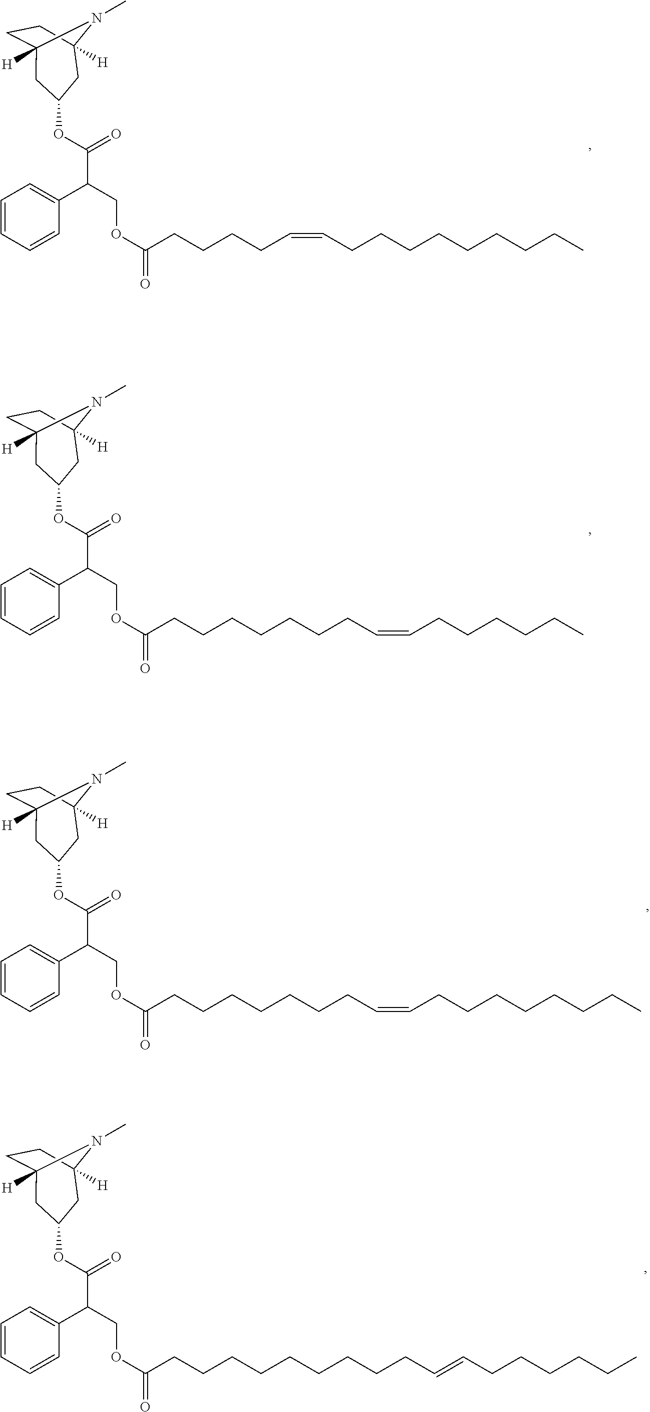 [SMILES: CCCCCCCCC/C=C\CCCCC(=O)OCC(C(=O)O[C@H]1C[C@H]2CC[C@@H](C1)N2C)c1ccccc1 ; CCCCCC/C=C\CCCCCCCC(=O)OCC(C(=O)O[C@H]1C[C@H]2CC[C@@H](C1)N2C)c1ccccc1 ; CCCCCCCC/C=C\CCCCCCCC(=O)OCC(C(=O)O[C@H]1C[C@H]2CC[C@@H](C1)N2C)c1ccccc1 ; CCCCCC/C=C/CCCCCCCCCC(=O)OCC(C(=O)O[C@H]1C[C@H]2CC[C@@H](C1)N2C)c1ccccc1]

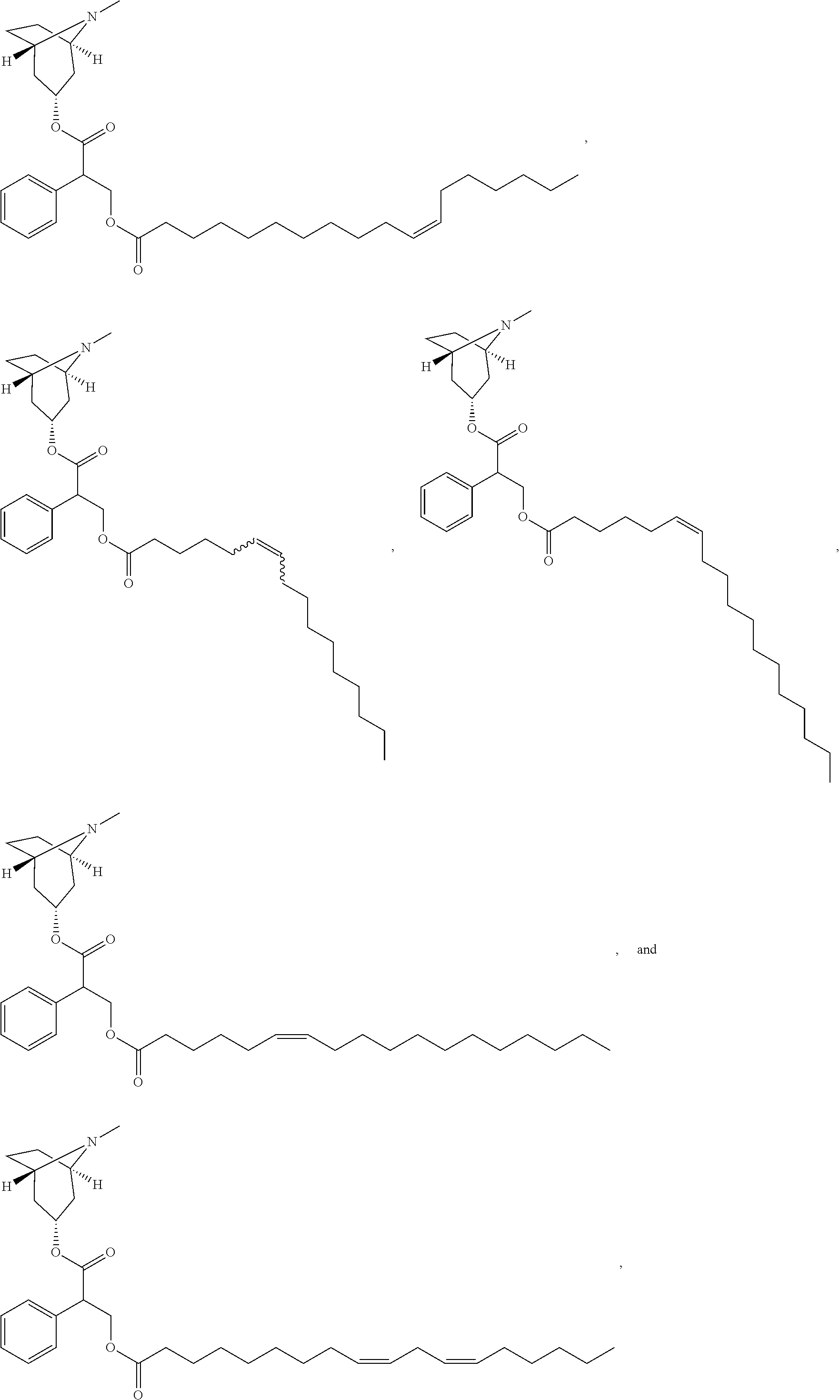 [SMILES: CCCCCC/C=C\CCCCCCCCCC(=O)OCC(C(=O)O[C@H]1C[C@H]2CC[C@@H](C1)N2C)c1ccccc1 ; CCCCCCCCCC=CCCCCC(=O)OCC(C(=O)O[C@H]1C[C@H]2CC[C@@H](C1)N2C)c1ccccc1 ; CCCCCCCCCCC/C=C\CCCCC(=O)OCC(C(=O)O[C@H]1C[C@H]2CC[C@@H](C1)N2C)c1ccccc1 ; CCCCCCCCCCC/C=C\CCCCC(=O)OCC(C(=O)O[C@H]1C[C@H]2CC[C@@H](C1)N2C)c1ccccc1 ; CCCCC/C=C\C/C=C\CCCCCCCC(=O)OCC(C(=O)O[C@H]1C[C@H]2CC[C@@H](C1)N2C)c1ccccc1]

or a pharmaceutically acceptable salt thereof.

In some embodiments, the compound of Formula (I) is selected from any one of the following compounds:

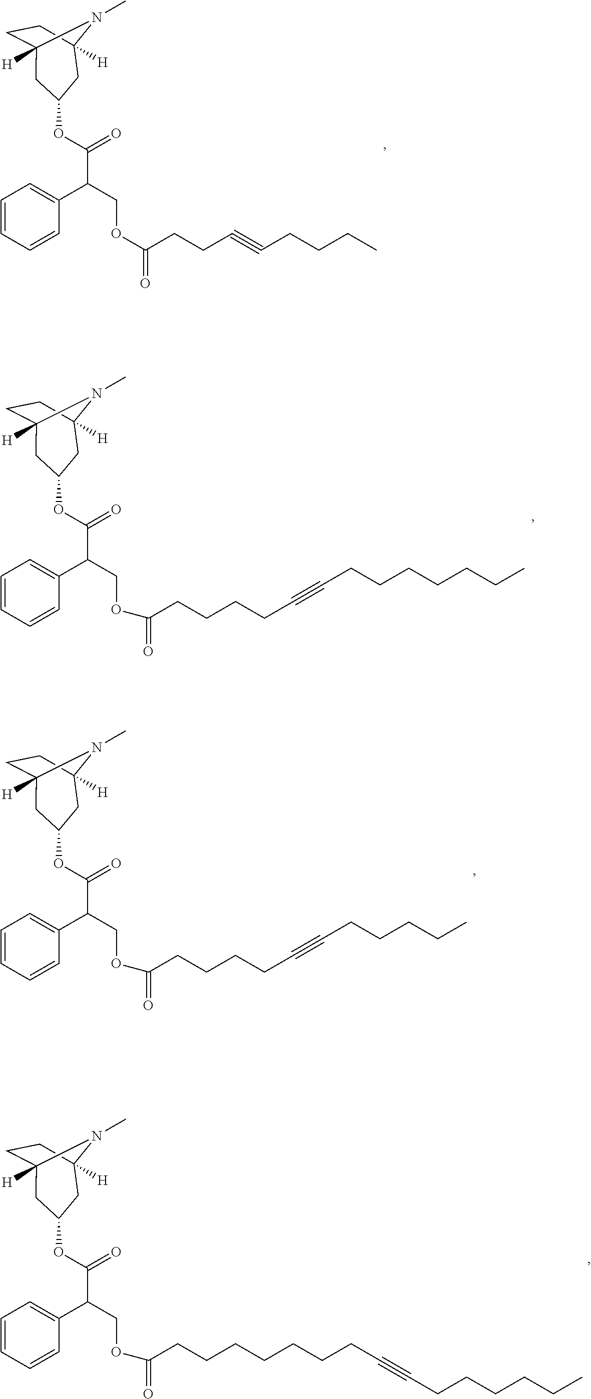 [SMILES: CCCCC#CCCC(=O)OCC(C(=O)O[C@H]1C[C@H]2CC[C@@H](C1)N2C)c1ccccc1 ; CCCCCCCC#CCCCCC(=O)OCC(C(=O)O[C@H]1C[C@H]2CC[C@@H](C1)N2C)c1ccccc1 ; CCCCCC#CCCCCC(=O)OCC(C(=O)O[C@H]1C[C@H]2CC[C@@H](C1)N2C)c1ccccc1 ; CCCCCCC#CCCCCCCCC(=O)OCC(C(=O)O[C@H]1C[C@H]2CC[C@@H](C1)N2C)c1ccccc1]

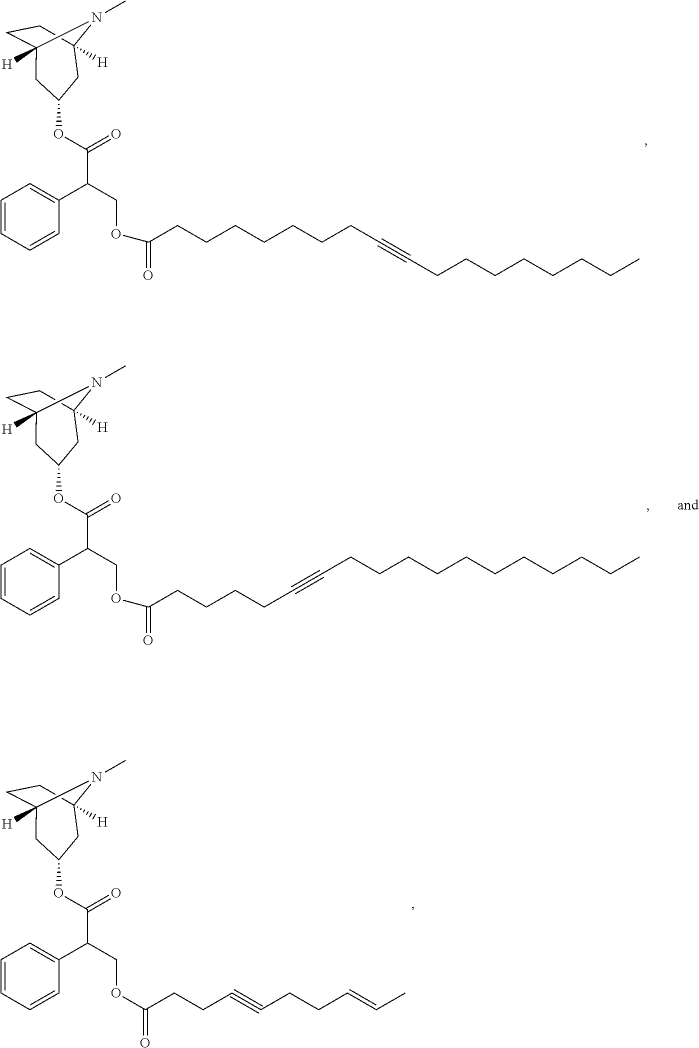 [SMILES: CCCCCCCCC#CCCCCCCCC(=O)OCC(C(=O)O[C@H]1C[C@H]2CC[C@@H](C1)N2C)c1ccccc1 ; CCCCCCCCCCCC#CCCCCC(=O)OCC(C(=O)O[C@H]1C[C@H]2CC[C@@H](C1)N2C)c1ccccc1 ; C/C=C/CCC#CCCC(=O)OCC(C(=O)O[C@H]1C[C@H]2CC[C@@H](C1)N2C)c1ccccc1]

or a pharmaceutically acceptable salt thereof.

In some embodiments, the compound of Formula (I) is selected from any one of the following compounds:

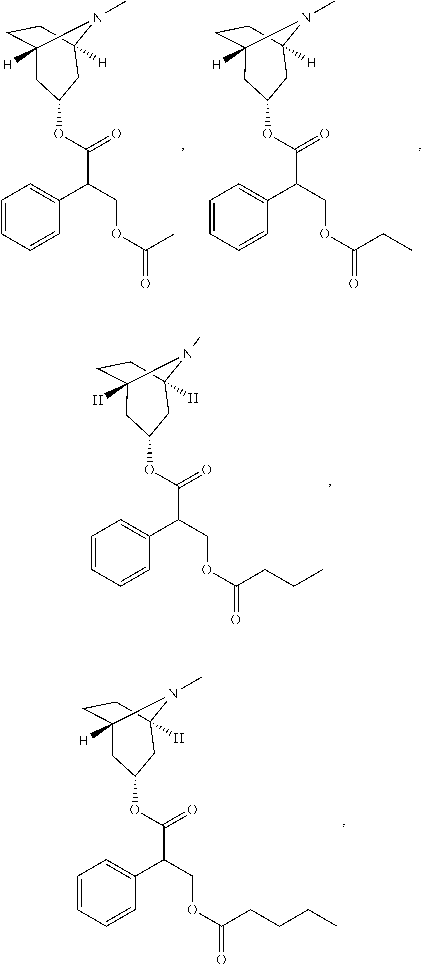

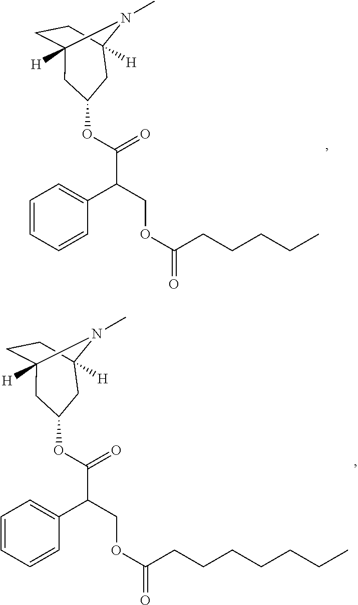 [SMILES: CCCCCC(=O)OCC(C(=O)O[C@H]1C[C@H]2CC[C@@H](C1)N2C)c1ccccc1 ; CCCCCCCC(=O)OCC(C(=O)O[C@H]1C[C@H]2CC[C@@H](C1)N2C)c1ccccc1]

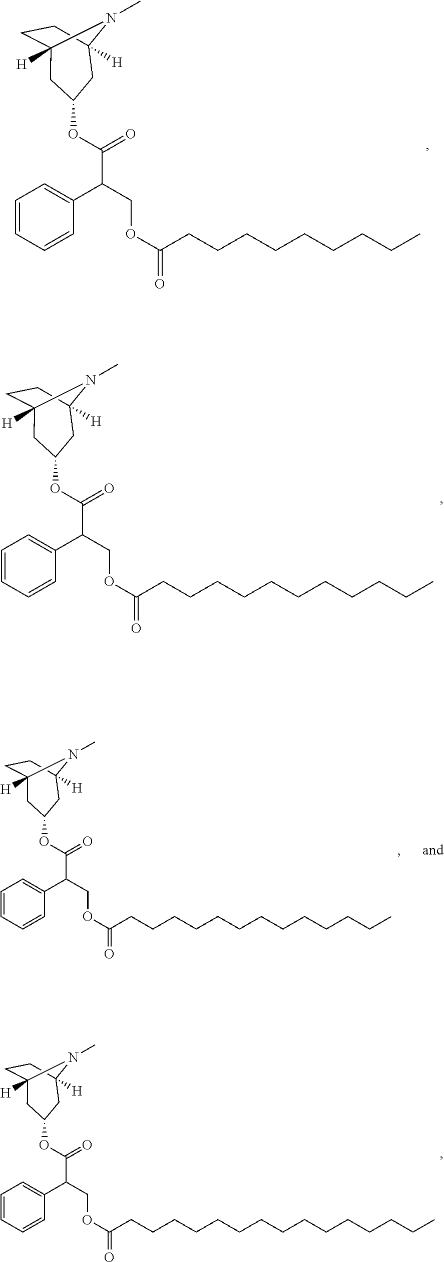 [SMILES: CCCCCCCCCC(=O)OCC(C(=O)O[C@H]1C[C@H]2CC[C@@H](C1)N2C)c1ccccc1 ; CCCCCCCCCCCC(=O)OCC(C(=O)O[C@H]1C[C@H]2CC[C@@H](C1)N2C)c1ccccc1 ; CCCCCCCCCCCCCC(=O)OCC(C(=O)O[C@H]1C[C@H]2CC[C@@H](C1)N2C)c1ccccc1 ; CCCCCCCCCCCCCCCC(=O)OCC(C(=O)O[C@H]1C[C@H]2CC[C@@H](C1)N2C)c1ccccc1]

or a pharmaceutically acceptable salt thereof

### Pharmaceutically Acceptable Salts

In some embodiments, a salt of a compound of Formula (I) is formed between an acid and a basic group of the compound, such as an amino functional group, or a base and an acidic group of the compound, such as a carboxyl functional group. According to another embodiment, the compound is a pharmaceutically acceptable acid addition salt. The salts retain the desired biological activity of the above identified compounds and exhibit minimal or no undesired toxicological effects.

The acid addition salt form of a compound of Formula (I) can be obtained by treating the free base with an appropriate acid such as an inorganic acid, e.g., hydrochloric acid, hydrobromic acid, sulfuric acid, phosphoric acid, nitric acid and the like; or an organic acid such as for example, acetic acid, hydroxyacetic acid, propanoic acid, lactic acid, pyruvic acid, malonic acid, fumaric acid, maleic acid, oxalic acid, tartaric acid, succinic acid, malic acid, ascorbic acid, benzoic acid, tannic acid, pamoic acid, citric acid, methylsulfonic acid, ethanesulfonic acid, benzenesulfonic acid, formic and the like (Handbook of Pharmaceutical Salts, P. Heinrich Stahal & Camille G. Wermuth (Eds), Verlag Helvetica Chemica Acta-Zurich, 2002, 329-345).

Compounds of Formula (I) and their salts can be in the form of a solvate, which is included within the scope of the present invention. Such solvates include for example hydrates, alcoholates (e.g., ethanol solvate), and the like. Compounds according to the present invention may exist in different polymorphic forms. Although not explicitly indicated in the above formula, such forms are intended to be included within the scope of the present disclosure.

### Methods of Making

Compounds of Formula (I), including salts thereof, can be prepared using known organic synthesis techniques and can be synthesized according to any of numerous possible synthetic routes. For example, the compounds described herein may be prepared using methods and procedures similar to those of Examples 1-2 herein. A person skilled in the art knows how to select and implement appropriate synthetic protocols, and appreciates that the processes described are not the exclusive means by which compounds provided herein may be synthesized, and that a broad repertoire of synthetic organic reactions is available to be potentially employed in synthesizing compounds provided herein.

Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The synthetic scheme set forth below, illustrates how compounds according to the invention can be made (wherein R¹ refers to any of the R¹ groups described herein for the compound of Formula (I)):

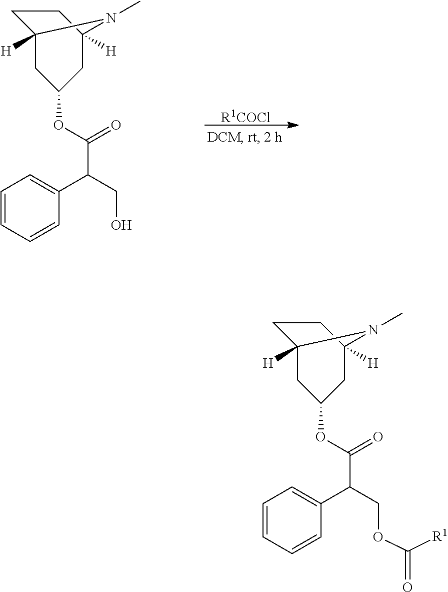

Those skilled in the art will be able to routinely modify and/or adapt the following scheme to synthesize any compounds of the invention covered by Formula I. The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4^{th} Ed., Wiley & Sons, Inc., New York (2006).

### Pharmaceutical Compositions and Formulations

The instant disclosure provides a pharmaceutical composition comprising an effective amount of a compound of the present disclosure or a pharmaceutically acceptable salt thereof; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament. The pharmaceutical composition may also comprise any one of the additional therapeutic agents described herein.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

In some embodiments, the pharmaceutically acceptable carrier is a semifluorinated alkane fluid. In one example, a partially fluorinated alkane compound useful in the formulations of this disclosure has a formula CF₃(CF₂)ₘ(CH₂)ₙCH₃. In some embodiments, m is an integer from 1 to 10, and n is an integer from 1 to 10. For example, m is can be 1, 2, 3, 4, 5, or 6. In another example, n can be 1, 2, 3, 4, 5, or 6. Suitable examples of partially fluorinated alkane compounds include perfluorohexyloctane (F6H8), perfluorohexylhexane (F6H6), perfluorobutylbutane (F4H4), and perfluorohexylethane (F6H2). In some embodiments, the concentration of the compound within the instant claims in a pharmaceutical formulation comprising partially fluorinated alkane vehicle liquid is from about 1 mg/mL to about 10 mg/mL. For example, the concentration is about 1 mg/mL, about 2 mg/mL, about 3 mg/mL, about 4 mg/mL, about 5 mg/mL, about 6 mg/mL, or about 7 mg/mL. In one example, the formulation comprising a semifluorinated alkane fluid as a pharmaceutically acceptable carrier may be any one of the ophthalmic formulations described herein. In another example, the formulation comprising a semifluorinated alkane fluid as a pharmaceutically acceptable carrier is an injectable composition suitable for intramuscular, intravenous, or subcutaneous administration.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Suitable examples of acceptable routes of administration include buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

See, for example, U.S. Pat. No. 6,803,031. Additional formulations and methods for intranasal administration are found in Ilium, L., J Pharm Pharmacol, 56, 3-17, 2004 and Ilium, L., Eur J Pharm Sci, 11, 1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, or soap. In some embodiments, the composition is in any form commonly employed in the art of cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

In some embodiments, the topical composition is an ophthalmic formulation, e.g., for intraocular administration. Suitable examples of ophthalmic formulations include eye drops, saline drops, suspensions, ointments, emulsions, nanoemulsions, oil, gel, hydrogel, solutions, Any one of these ophthalmic formulations can be administered directly to the cornea, pupil, retina, choroid, sclera, and/or iris of the eye, using, for example, a plastic or latex applicator (e.g., a single-use applicator), an eye dropper, a glass pipette, or a rubber bulb. These formulations may contain, for example, a partially fluorinated alkane liquid vehicle, as described above. In addition, the ophthalmic formulation may contain water, saline, a dextrose solution, a cellulose or its derivative, such as hydroxypropylmethylcellulose, a surfactant such as polysorbate 20, a chelating agent, or a cyclodextrin.

### Dosages and Regimens

In the pharmaceutical compositions of the present application, a compound of the present disclosure (e.g., a compound of Formula (I)) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound in the ophthalmic formulation (e.g., Formula (I) or a pharmaceutically acceptable salt thereof) can range, for example, from about 0.0001% to about 10% (e.g., from about 0.0001% to about 1%; from about 0.0010% to about 10%; from about 0.010% to about 10%; from about 0.10% to about 1%). In some embodiments, an effective amount of a compound of Formula (I) is about 0.001%, about 0.010%, about 0.10%, or about 0.10%, or about 10%.

In some embodiments, an effective amount (e.g., in the systemic formulation) of the compound (e.g., Formula (I) or a pharmaceutically acceptable salt thereof) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, two times daily, three times daily, or four times daily) or non-daily basis (e.g., every other day, every two days, every three days, once weekly, twice weekly, once every two weeks, once a month).

### Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

### Combinations

The compounds of the present disclosure can be used on combination with at least one medication or therapy useful, e.g., in treating or preventing a disease is which a muscarinic acetylcholine receptor is implicated. Suitable examples of such medications include muscarinic acetylcholine receptor agonists and/or antagonists, or pharmaceutically acceptable salts thereof.

Suitable examples of muscarinic receptor agonists include acetylcholine, arecoline, oxotremorine, muscarine, carbachol, nebracetam, bethanechol, pilocarpine, and desmethylclozapine, or a pharmaceutically acceptable salt thereof. Suitable examples of muscarinic receptor antagonists include atropine, hyoscyamine, scopolamine, diphenhydramine, dimenhydrinate, dicycloverine, tolterodine, oxybutynin, ipratropium, pirenzepine, telenzepine, tripitramine, gallamine, methoctramine, darifenacin, tiotropium, chlorpromazine, and haloperidol, or a pharmaceutically acceptable salt thereof.

In some embodiments, the compound of formula (I) can be administered in combination with a cycloplegic agent, for example, atropine, cyclopentolate, homatropine, scopolamine, or tropicamide, or a pharmaceutically acceptable salt thereof. In some embodiments, the compound of formula (I) can be administered in combination with a mydriatic, such as tropicamide, phenylephrine, cyclopentolate, atropine, scopolamine, or homatropine, or a pharmaceutically acceptable salt thereof

In some embodiments, the compound of formula (I) can be administered in combination with an agent to treat bradycardia, such as atropine or another anticholinergic, an inotrope (dopamine, dobutaime, dopexamine, adrenaline, isoproterenol, noradrenaline, or a pharmaceutically acceptable salt thereof).

In some embodiments, the compound of formula (I) can be administered in combination with an agent for hyperhidrosis, such as formaldehyde, aluminium chlorohydrate, or an anticholinergic (propantheline, glycopyrronium bromide or glycopyrrolate, oxybutynin, methantheline, or benzatropine).

In some embodiments, the compound of formula (I) can be administered in combination with an antidote to poisoning, such as atropine, benztropine, botulinum antitoxin, cysteine, bicarbonate, glucagon, or pyridoxine, or a pharmaceutically acceptable salt thereof.

The compound of the present disclosure may be administered to the patient simultaneously with the additional therapeutic agent (in the same pharmaceutical composition or dosage form or in different compositions or dosage forms) or consecutively (the additional therapeutic agent may be administered in a separate pharmaceutical composition or dosage form before or after administration of the compound of the present disclosure).

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

The term “alkyl,” as used herein, refers to saturated, monovalent hydrocarbon moieties having linear or branched moieties or combinations thereof and containing the specified (e.g., 2 to 22) carbon atoms. One methylene (—CH₂—) group, of the alkyl can be replaced by oxygen, sulfur, sulfoxide, nitrogen, carbonyl, carboxyl, sulfonyl, or by a divalent C₃₋₈ cycloalkyl. Alkyl groups can be independently substituted by halogen atoms, hydroxyl groups, cycloalkyl groups, amine groups, heterocyclic groups, carboxylic acid groups, phosphonic acid groups, sulphonic acid groups, phosphoric acid groups, nitro groups, amide groups, sulfonamides groups.

The term “alkenyl,” as used herein, refers to a monovalent or divalent hydrocarbon radical having the specified (e.g., 2 to 22) carbon atoms, derived from a saturated alkyl, having at least one double bond (e.g., one, two, three, four, five, or six double bonds). The alkenyl can be in the E or Z configuration (e.g., each of the double bonds in the alkenyl can independently be in E or Z configuration). Alkenyl groups can be substituted by C₁₋₈ alkyl.

The term “alkynyl,” as used herein, refers to a monovalent or divalent hydrocarbon radical having the specified (e.g., 2 to 20) carbon atoms, derived from a saturated alkyl, having at least one triple bond (e.g., one, two, three, four, five, or six triple bonds). Alkynyl groups can optionally be substituted by C₁₋₈ alkyl.

The term “cycloalkyl,” as used herein, refers to a monovalent or divalent group of, e.g., 3 to 8 carbon atoms, derived from a saturated cyclic hydrocarbon. Cycloalkyl groups can be monocyclic or polycyclic. Cycloalkyl can be independently substituted by halogen, nitro groups, cyano groups, —OC₁₋₆ alkyl groups, —SC₁₋₆ alkyl groups, —C₁₋₆ alkyl groups, —C₂₋₆ alkenyl groups, —C₂₋₆ alkynyl groups, C₃₋₈ cycloalkyl groups, carboxylic acid groups, ester groups, ketone groups, aldehyde groups, amide groups, amine groups, sulfonamide groups or hydroxyl groups.

The term “cycloalkenyl,” as used herein, refers to a monovalent or divalent group of e.g. 3 to 8 carbon atoms, derived from a saturated cycloalkyl having at least one double bond and/or at least one triple bond. Cycloalkenyl groups can be monocyclic or polycyclic. Cycloalkenyl groups can be independently substituted by halogen atoms, nitro groups, cyano groups, —OC₁₋₆ alkyl groups, —SC₁₋₆ alkyl groups, —C₁₋₆ alkyl groups, —C₂₋₆ alkenyl groups, —C₂₋₆ alkynyl groups, carboxylic acid groups, ester groups, ketone groups, aldehyde groups, amide groups, amine groups, sulfonamide groups, C₃₋₈ cycloalkyl groups or hydroxyl groups.

The term “heterocycle,” as used herein, refers to a 3 to 10 membered ring, which can be aromatic or non-aromatic, saturated or non-saturated, monocyclic or polycyclic, containing at least one heteroatom selected form O or N or S or combinations of at least two thereof, interrupting the carbocyclic ring structure. The heterocyclic ring can be interrupted by a C═O; the S heteroatom can be oxidized. Heterocycles can be monocyclic or polycyclic. Heterocyclic ring moieties can be substituted by halogen, nitro groups, cyano groups, —OC₁₋₆ alkyl groups, —SC₁₋₆ alkyl groups, —C₁₋₆ alkyl groups, —C₂₋₆ alkenyl groups, —C₂₋₆ alkynyl groups, carboxylic acid groups, ester groups, ketone groups, aldehyde groups, amide groups, amine groups, sulfonamide groups, C₃₋₈ cycloalkyl groups or hydroxyl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

The term “aryl” as used herein, refers to an organic moiety derived from an aromatic hydrocarbon consisting of a ring containing 6 to 10 carbon atoms by removal of one hydrogen. Aryl can be monocyclic or polycyclic. Aryl can be substituted by halogen atoms, nitro groups, cyano groups, —OC₁₋₆ alkyl groups, —SC₁₋₆ alkyl groups, —C₁₋₆ alkyl groups, —C₂₋₆ alkenyl groups, —C₂₋₆ alkynyl groups, carboxylic acid groups, ester groups, ketone groups, aldehyde groups, amide groups, amine groups, sulfonamide groups, C₃₋₈ cycloalkyl groups or hydroxyl groups. Usually aryl is phenyl. Preferred substitution site on aryl are meta and para positions.

The term “cyano” as used herein, represents a group of formula “—CN”.

The term “nitro” as used herein, represents a group of formula “—NO₂”.

The term “amide” as used herein, represents a group of formula “—C(O)NR^{x}R^{y},” wherein R^{x} and R^{y} can be the same or independently H, alkyl, aryl, cycloalkyl, cycloalkenyl, heterocyle as defined above.

The term “amine” or “amino” as used herein, represents a group of formula “—NR^{x}R^{y}”, wherein R^{x} and R^{y} can be the same or independently H, alkyl, aryl, cycloalkyl, cycloalkenyl, heterocyle as defined above.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

The term “ketone” as used herein, represents an organic compound having a carbonyl group linked to a carbon atom such as —(CO)R^{x} wherein R^{x} can be alkyl, aryl, cycloalkyl, cycloalkenyl, heterocyle as defined above.

The term “aldehyde” as used herein, represents a group of formula “—C(O)H”.

The term “ester” as used herein, represents a group of formula “—C(O)OR^{x}”, wherein R^{x} can be alkyl, aryl, cycloalkyl, cycloalkenyl, heterocyle as defined above.

The term “sulfonamide” as used herein, represents a group of formula “—S(O)₂NR^{X}R” wherein R^{x} and R^{y} can be the same or independently H, alkyl, aryl, cycloalkyl, cycloalkenyl, heterocyle as defined above.

The term “sulfoxide” as used herein, represents a group of formula “—S═O”.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, “halo” refers to F, Cl, Br, or I. In some embodiments, a halo is F, C₁, or Br.

As used herein, the term “carboxy” refers to a —C(O)OH group.

As used herein, the term “carbamyl” to a group of formula —C(O)NH₂. As used herein, the term “aminosulfonyl” refers to a group of formula —S(O)₂NH₂. As used herein, the term “aminosulfonylamino” refers to a group of formula —NHS(O)₂NH₂.

The formula “H”, as used herein, represents a hydrogen atom.

The formula “O”, as used herein, represents an oxygen atom.

The formula “N”, as used herein, represents a nitrogen atom.

The formula “S”, as used herein, represents a sulfur atom.

The term “compound” as used herein is meant to include all stereoisomers, geometric isomers, tautomers, and isotopes of the structures depicted, including racemic mixtures. Compounds herein identified by name or structure as one particular tautomeric form are intended to include other tautomeric forms unless otherwise specified. Compound names were generated using software such as Chem Bio Draw Ultra version 14.0, and the name accuracy was not further verified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration. Some compounds of Formula I and some of their intermediates have at least one stereogenic center in their structure. This stereogenic center may be present in an R or S configuration, said R and S notation is used in correspondence with the rules described in Pure Appli. Chem. (1976), 45, 11-13.

The present invention includes all pharmaceutically acceptable isotopically enriched compounds. Any compound of the invention may contain one or more isotopic atoms enriched or different than the natural ratio such as deuterium ²H (or D) in place of protium ¹H (or H) or use of ¹³C enriched material in place of ¹²C and the like. Similar substitutions can be employed for N, O and S. The use of isotopes may assist in analytical as well as therapeutic aspects of the invention. For example, use of deuterium may increase the in vivo half-life by altering the metabolism (rate) of the compounds of the invention. These compounds can be prepared in accord with the preparations described by use of isotopically enriched reagents.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lacta-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the eye tissue with a compound of the invention includes the administration of a compound of the present invention to an individual or patient, such as a human, having the eye tissue in need of treatment, as well as, for example, introducing a compound of the invention into a sample containing a cellular or purified preparation containing the ocular tissue.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans. In some embodiment, a human subject may be of any age, for example, from 0 to 100 years old. As used herein, “pediatric subject” and “child” are used interchangeably and refer to subjects from 0 to 18 years old, for example, 1, 2, 5, 10, 12, or 14 years old.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### Numbered Embodiments

The compounds of Formula (I), pharmaceutically acceptable salts thereof, compositions (including pharmaceutical compositions) comprising same, as well as the methods of their use, may be described with reference to the following numbered Embodiments.

Embodiment 1 is a compound of Formula (I):

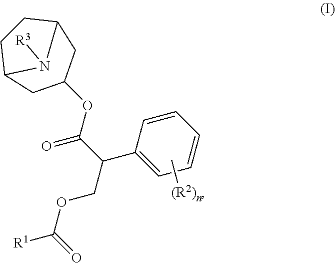

or a pharmaceutically acceptable salt thereof, wherein:
R³ is selected from H, C₁₋₆ alkyl, and C₁₋₆ haloalkyl;
each R² is independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino;
n is selected from 0, 1, 2, 3, 4, and 5; and
R¹ is selected from C₇₋₃₄ alkyl, C₇₋₃₄ alkenyl, C₇₋₃₄ alkynyl, C₃₋₁₀ cycloalkyl, 4-10 membered heterocycloalkyl, 5-14 membered heteroaryl, and C₆₋₁₀ aryl, wherein said C₇₋₃₄ alkyl, C₇₋₃₄ alkenyl, and C₇₋₃₄ alkynyl are each optionally substituted with 1 or 2 substituents independently selected from C₃₋₁₀ cycloalkyl, 4-10 membered heterocycloalkyl, 5-14 membered heteroaryl, and C₆₋₁₀ aryl, and each of said C₃₋₁₀ cycloalkyl, 4-10 membered heterocycloalkyl, 5-14 membered heteroaryl, and C₆₋₁₀ aryl is optionally substituted with 1, 2, or 3 substituents independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino;
with a proviso that the compound of Formula (I) is not atropine oleate or hyoscyamine oleate.

Embodiment 2 is the compound of Embodiment 1, having formula:

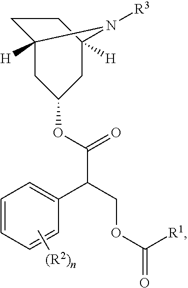

or a pharmaceutically acceptable salt thereof.

Embodiment 3 is the compound of Embodiment 1 or 2, wherein R³ is C₁₋₆ alkyl.

Embodiment 4 is the compound of any one of Embodiments 1-3, wherein n is selected from 1, 2, and 3.

Embodiment 5 is the compound of Embodiment 4, wherein R² is selected from C₁₋₃ alkyl, C₁₋₃ haloalkyl, C₁₋₃ alkoxy, C₁₋₃ haloalkoxy, OH, NO₂, CN, halo, and amino.

Embodiment 6 is the compound of any one of Embodiments 1-3, wherein n is 0.

Embodiment 7 is the compound of any one of Embodiments 1-6, wherein the compound of Formula (I) has formula:

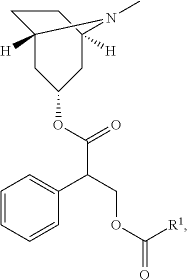

or a pharmaceutically acceptable salt thereof.

Embodiment 8 is the compound of any one of Embodiments 1-6, wherein the compound of Formula (I) has formula:

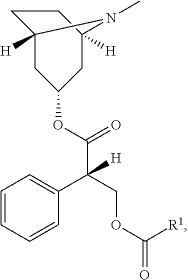

or a pharmaceutically acceptable salt thereof.

Embodiment 9 is the compound of any one of Embodiments 1-6, wherein the compound of Formula (I) has formula:

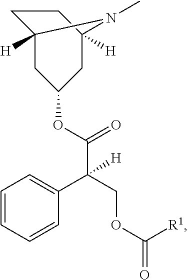

or a pharmaceutically acceptable salt thereof.

Embodiment 10 is the compound of any one of Embodiments 1-9, wherein R¹ is selected from C₇₋₂₁ alkyl, C₇₋₂₁ alkenyl, and C₇₋₂₁ alkynyl.

Embodiment 11 is the compound of any one of Embodiments 1-9, wherein R¹ is selected from C₇₋₃₄ alkenyl and C₇₋₃₄ alkynyl.

Embodiment 12 is the compound of any one of Embodiments 1-9, wherein R¹ is selected from C₇₋₂₁ alkenyl and C₇₋₂₁ alkynyl.

Embodiment 13 is the compound of Embodiment 12, wherein R¹ is C₇₋₂₁ alkenyl.

Embodiment 14 is the compound of Embodiment 13, wherein R¹ is C₇₋₂₁ alkenyl comprising one double bond.

Embodiment 15 is the compound of Embodiment 13, wherein R¹ is C₇₋₂₁ alkenyl comprising two double bonds.

Embodiment 16 is the compound of Embodiment 13, wherein R¹ is C₁₂₋₂₁ alkenyl comprising one double bond.

Embodiment 17 is the compound of Embodiment 13, wherein R¹ is C₁₂₋₂₁ alkenyl comprising two double bonds.

Embodiment 18 is the compound of Embodiment 12, wherein R¹ is C₇₋₂₁ alkynyl.

Embodiment 19 is the compound of any one of Embodiments 1-9, wherein R¹ is C₇₋₂₁ alkyl.

Embodiment 20 is the compound of Embodiment 1, wherein the compound is selected from any one of the following compounds:

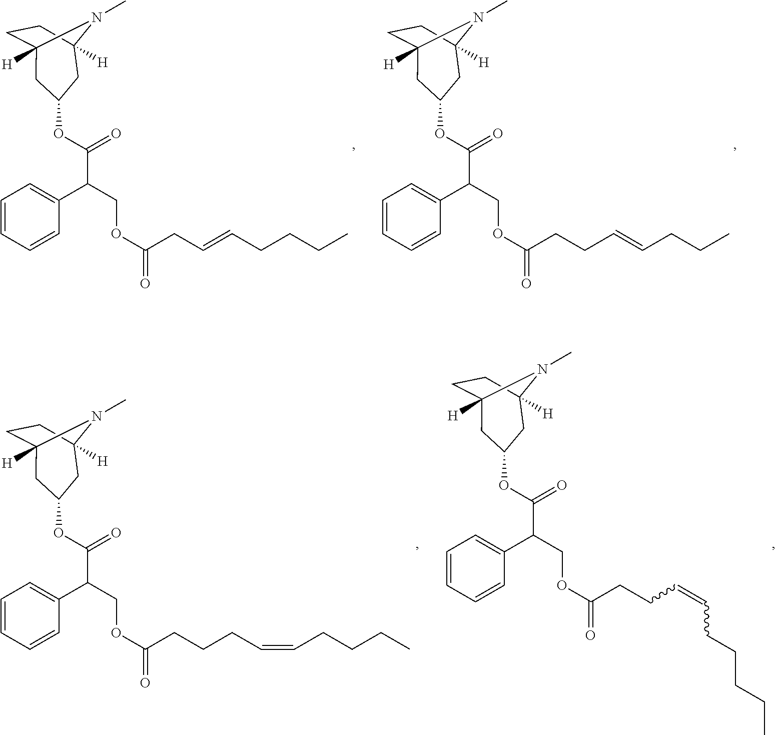

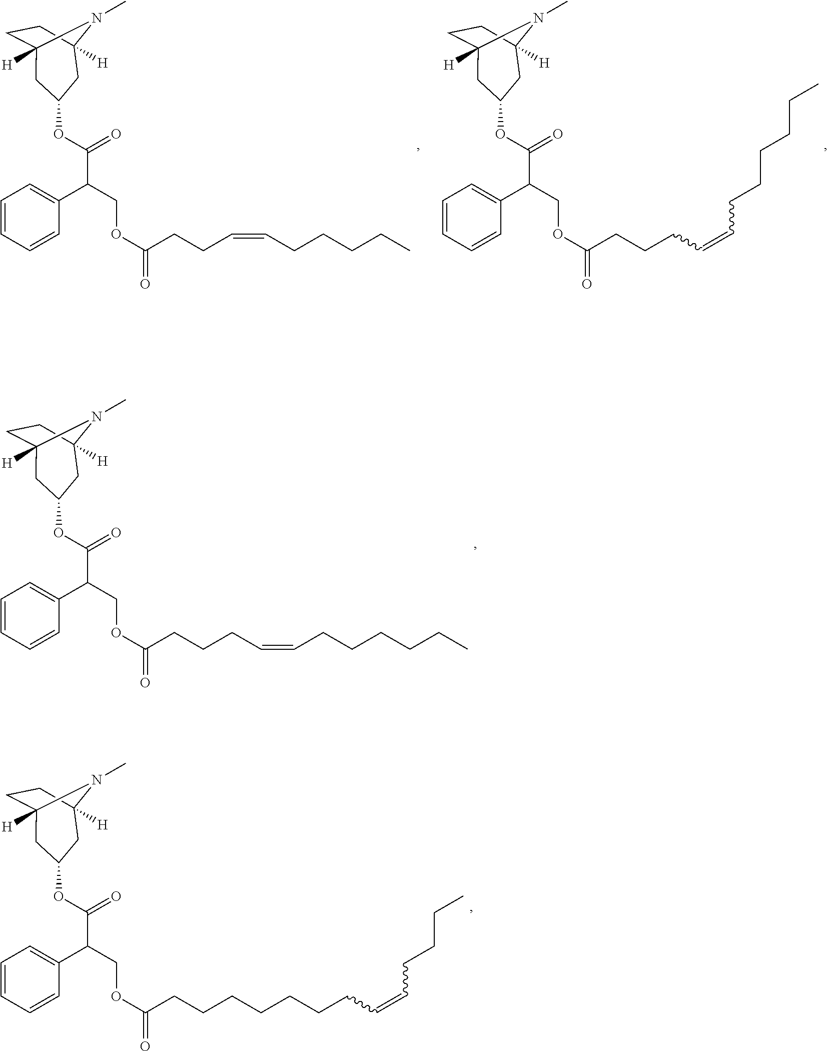

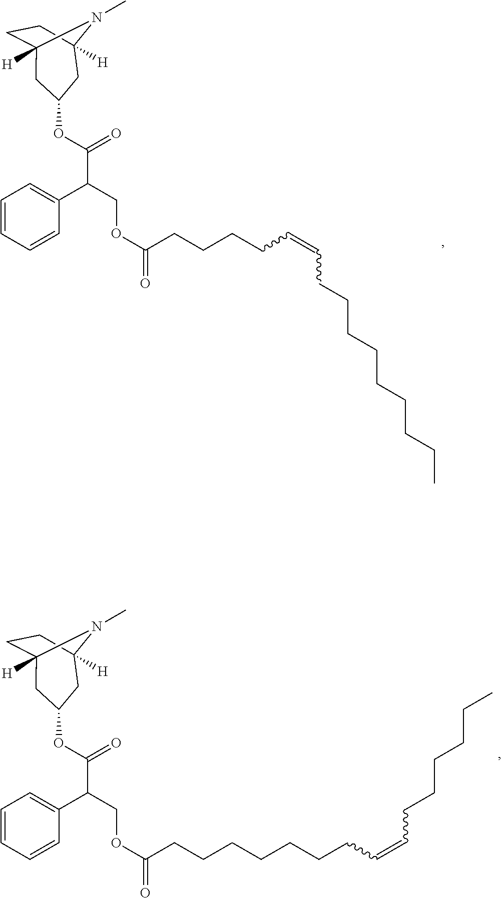 [SMILES: CCCCCCCCCC=CCCCCC(=O)OCC(C(=O)O[C@H]1C[C@H]2CC[C@@H](C1)N2C)c1ccccc1 ; CCCCCCC=CCCCCCCCC(=O)OCC(C(=O)O[C@H]1C[C@H]2CC[C@@H](C1)N2C)c1ccccc1]

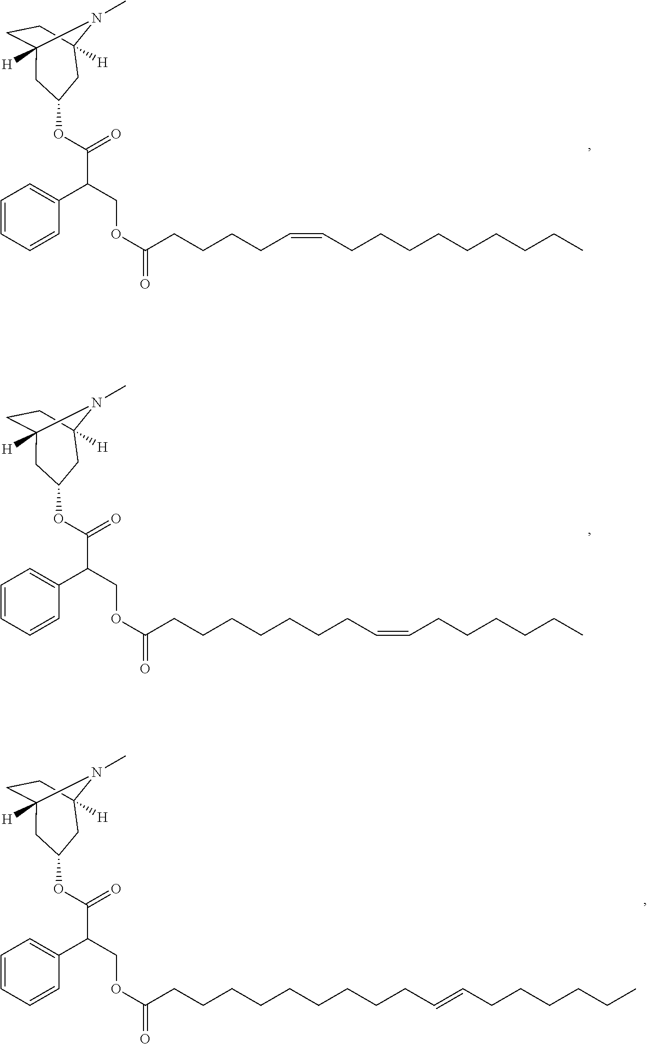 [SMILES: CCCCCCCCC/C=C\CCCCC(=O)OCC(C(=O)O[C@H]1C[C@H]2CC[C@@H](C1)N2C)c1ccccc1 ; CCCCCC/C=C\CCCCCCCC(=O)OCC(C(=O)O[C@H]1C[C@H]2CC[C@@H](C1)N2C)c1ccccc1 ; CCCCCC/C=C/CCCCCCCCCC(=O)OCC(C(=O)O[C@H]1C[C@H]2CC[C@@H](C1)N2C)c1ccccc1]

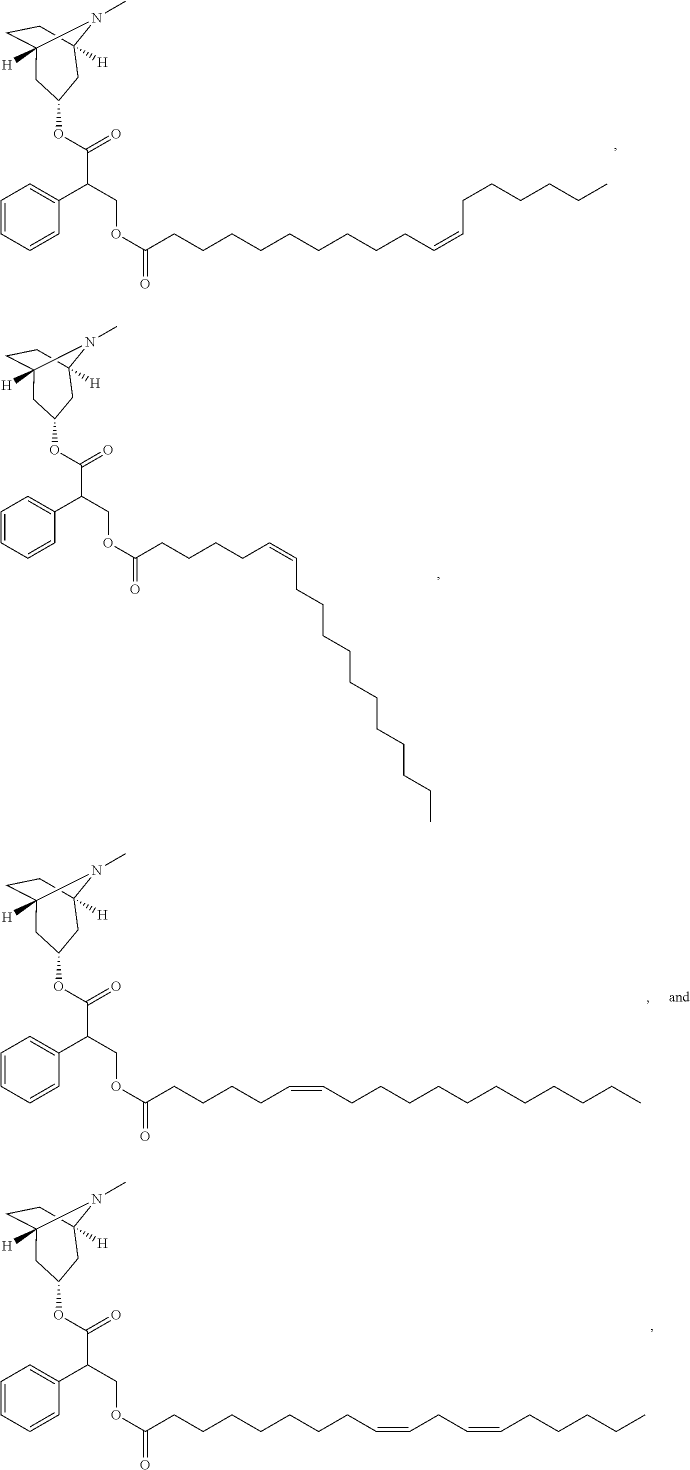 [SMILES: CCCCCC/C=C\CCCCCCCCCC(=O)OCC(C(=O)O[C@H]1C[C@H]2CC[C@@H](C1)N2C)c1ccccc1 ; CCCCCCCCCCC/C=C\CCCCC(=O)OCC(C(=O)O[C@H]1C[C@H]2CC[C@@H](C1)N2C)c1ccccc1 ; CCCCCCCCCCC/C=C\CCCCC(=O)OCC(C(=O)O[C@H]1C[C@H]2CC[C@@H](C1)N2C)c1ccccc1 ; CCCCC/C=C\C/C=C\CCCCCCCC(=O)OCC(C(=O)O[C@H]1C[C@H]2CC[C@@H](C1)N2C)c1ccccc1]

or a pharmaceutically acceptable salt thereof.

Embodiment 21 is the compound of Embodiment 1, selected from any one of the following compounds:

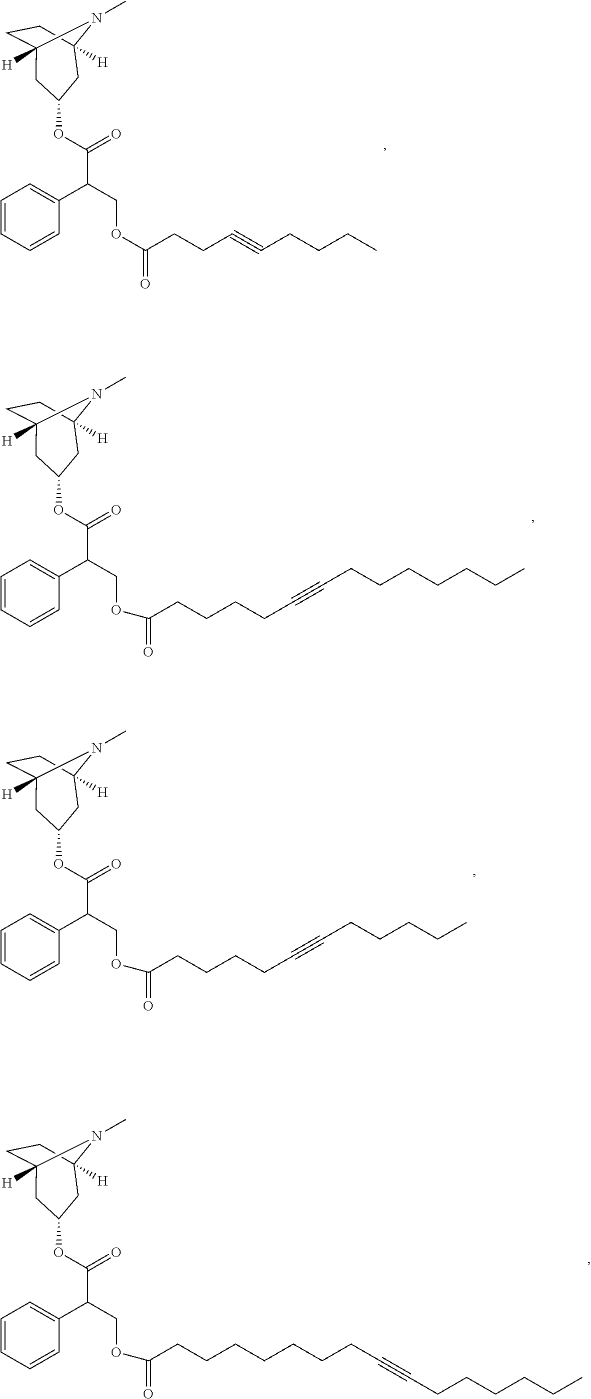 [SMILES: CCCCC#CCCC(=O)OCC(C(=O)O[C@H]1C[C@H]2CC[C@@H](C1)N2C)c1ccccc1 ; CCCCCCCC#CCCCCC(=O)OCC(C(=O)O[C@H]1C[C@H]2CC[C@@H](C1)N2C)c1ccccc1 ; CCCCCC#CCCCCC(=O)OCC(C(=O)O[C@H]1C[C@H]2CC[C@@H](C1)N2C)c1ccccc1 ; CCCCCCC#CCCCCCCCC(=O)OCC(C(=O)O[C@H]1C[C@H]2CC[C@@H](C1)N2C)c1ccccc1]

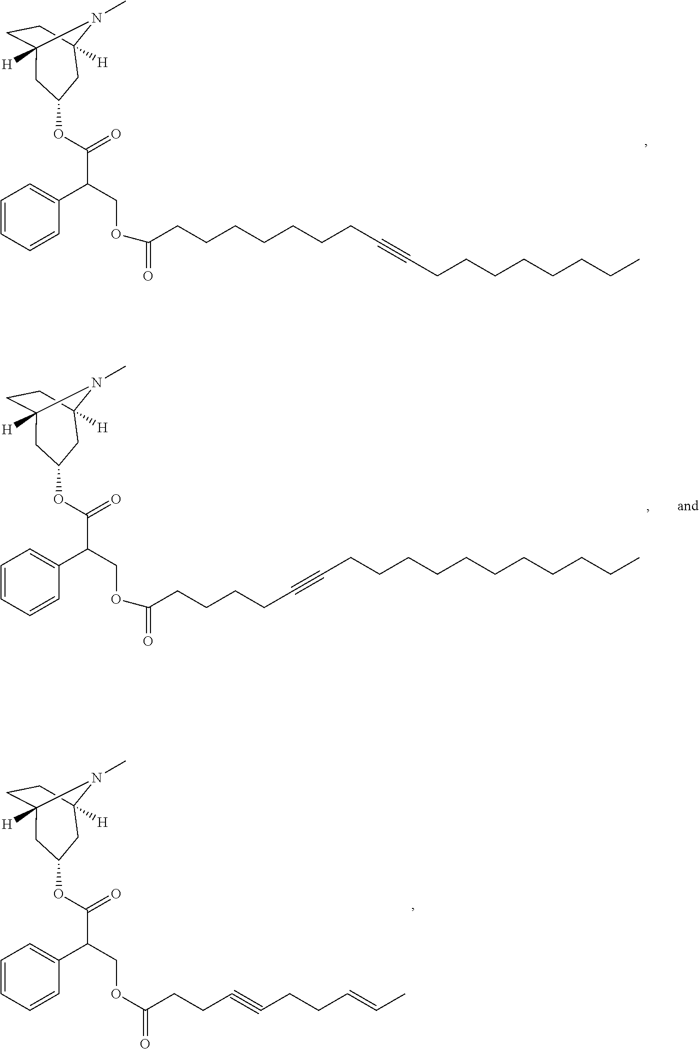 [SMILES: CCCCCCCCC#CCCCCCCCC(=O)OCC(C(=O)O[C@H]1C[C@H]2CC[C@@H](C1)N2C)c1ccccc1 ; CCCCCCCCCCCC#CCCCCC(=O)OCC(C(=O)O[C@H]1C[C@H]2CC[C@@H](C1)N2C)c1ccccc1 ; C/C=C/CCC#CCCC(=O)OCC(C(=O)O[C@H]1C[C@H]2CC[C@@H](C1)N2C)c1ccccc1]

or pharmaceutically acceptable salt thereof.

Embodiment 22 is the compound of Embodiment 1, wherein the compound is selected from any one of the following compounds:

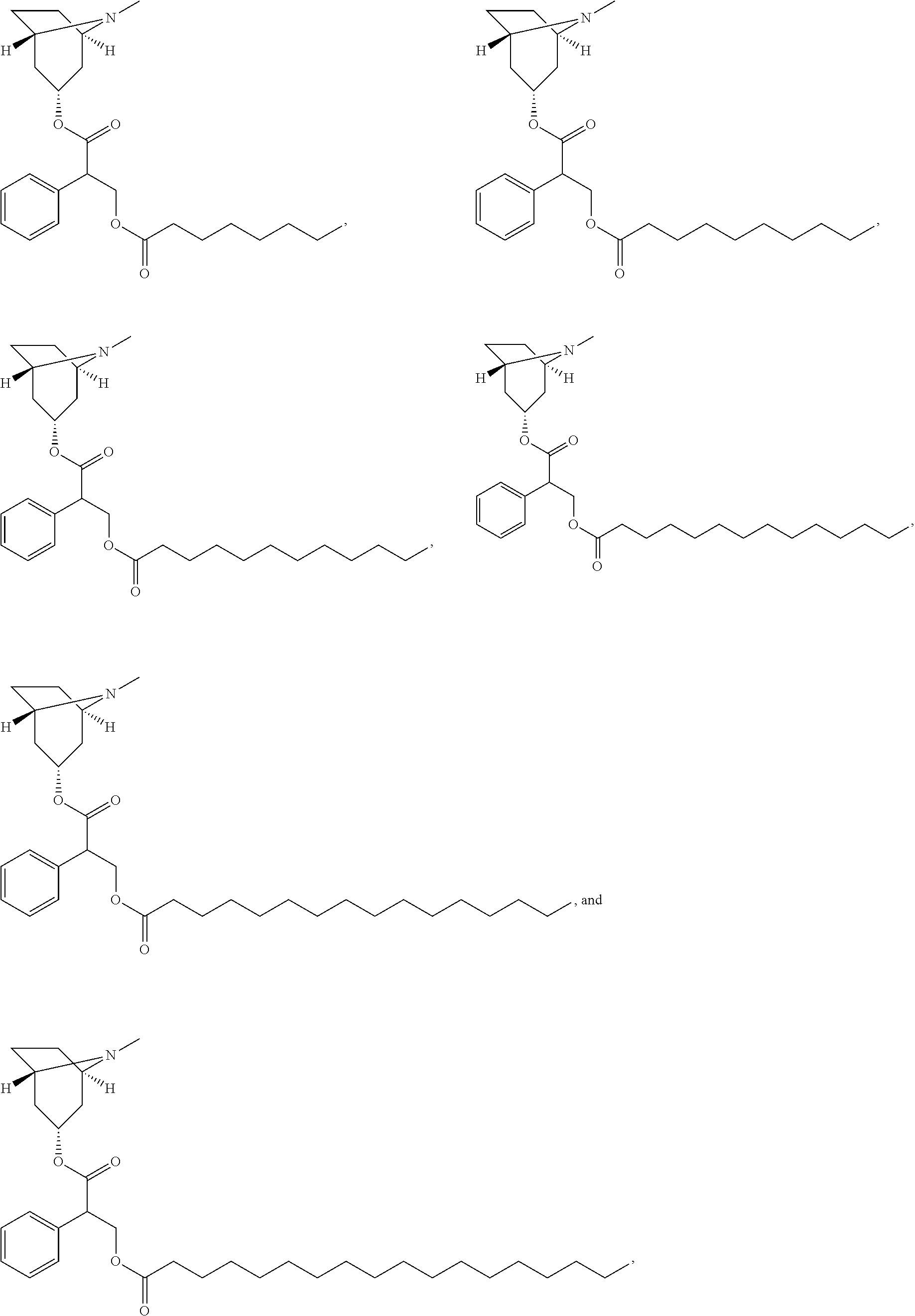

or a pharmaceutically acceptable salt thereof.

Embodiment 23 is a pharmaceutical composition comprising a compound of any one of Embodiments 1-22, or a pharmaceutically acceptable salt thereof, and a pharmaceutically acceptable carrier.

Embodiment 24 is the pharmaceutical composition of Embodiment 23, wherein the pharmaceutically acceptable carrier comprises a semifluorinated alkane.

Embodiment 25 is the pharmaceutical composition of Embodiment 24, wherein the semifluorinated alkane is selected from perfluorohexyloctane (F6H8), perfluorohexylhexane (F6H6), perfluorobutylbutane (F4H4), and perfluorohexylethane (F6H2).

Embodiment 26 is a method of treating or preventing a disease or condition where muscarinic acetylcholine receptor is implicated, the method comprising administering to a subject in need thereof a therapeutically effective amount of a compound of any one of Embodiments 1-22, or a pharmaceutically acceptable salt thereof, or a pharmaceutical composition of any one of Embodiments 23-25.

Embodiment 27 is the method of Embodiment 26, wherein the compound of any one of Embodiments 1-22, or a pharmaceutically acceptable salt thereof, is used for causing cycloplegic refraction in the eye of the subject.

Embodiment 28 is the method of Embodiment 26, wherein the compound of any one of Embodiments 1-22, or a pharmaceutically acceptable salt thereof, is used for causing mydriasis in the eye of the subject.

Embodiment 29 is the method of Embodiment 26, wherein the disease or condition is a painful ciliary muscle spasm in the eye.

Embodiment 30 is the method of Embodiment 26, wherein the compound of any one of Embodiments 1-22, or a pharmaceutically acceptable salt thereof, is used to relieve the eye floater symptoms.

Embodiment 31 is the method of Embodiment 26, wherein the compound of any one of Embodiments 1-22, or a pharmaceutically acceptable salt thereof, is used as a patching therapy for children with amblyopia or lazy eye.

Embodiment 32 is the method of Embodiment 26, wherein the disease or condition is myopia progression.

Embodiment 33 is the method of any one of Embodiments 27-32, wherein the pharmaceutical composition of any one of Embodiments 23-25 is an ophthalmic composition.

Embodiment 34 is the method of Embodiment 33, wherein the ophthalmic composition comprises eye drops comprising an emulsion, a suspension, or a solution of the compound of any one of Embodiments 1-22, or a pharmaceutically acceptable salt thereof.

Embodiment 35 is the method of Embodiment 26, wherein the disease or condition is a heart condition.

Embodiment 36 is the method of Embodiment 35, wherein the heart condition is a low heart rate (bradycardia).

Embodiment 37 is the method of Embodiment 26, wherein the compound of any one of Embodiments 1-22, or a pharmaceutically acceptable salt thereof, is used to reduce salivation and bronchial secretions.

Embodiment 38 is the method of Embodiment 26, wherein the disease or condition is hyperhidrosis.

Embodiment 39 is the method of Embodiment 26, wherein the disease or condition is poisoning.

Embodiment 40 is the method of any one of Embodiments 35-39, wherein the compound of any one of Embodiments 1-22, or a pharmaceutically acceptable salt thereof, is administered to the subject by oral, intramuscular, intravenous, subcutaneous, intraosseous, or endotracheal route, or by inhalation.

Embodiment 41 is a method for causing a condition selected from cycloplegic refraction and mydriasis in the eye of the subject, for relieving an eye floater symptom in a subject, for patching therapy for children with amblyopia or lazy eye, or for treating or preventing a condition selected from painful ciliary muscle spasm in the eye and a myopia progression, the method comprising administering to an eye of the subject an ophthalmic composition comprising a compound of Formula (I):

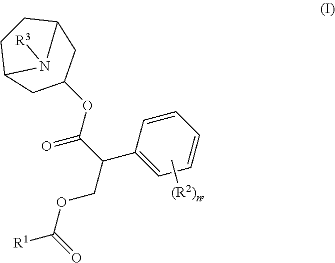

or a pharmaceutically acceptable salt thereof, wherein:
R³ is selected from H, C₁₋₆ alkyl, and C₁₋₆ haloalkyl;
each R² is independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino;
n is selected from 0, 1, 2, 3, 4, and 5; and
R¹ is selected from C₇₋₃₄ alkyl, C₇₋₃₄ alkenyl, C₇₋₃₄ alkynyl, C₃₋₁₀ cycloalkyl, 4-10 membered heterocycloalkyl, 5-14 membered heteroaryl, and C₆₋₁₀ aryl, wherein said C₇₋₃₄ alkyl, C₇₋₃₄ alkenyl, and C₇₋₃₄ alkynyl are each optionally substituted with 1 or 2 substituents independently selected from C₃₋₁₀ cycloalkyl, 4-10 membered heterocycloalkyl, 5-14 membered heteroaryl, and C₆₋₁₀ aryl, and each of said C₃₋₁₀ cycloalkyl, 4-10 membered heterocycloalkyl, 5-14 membered heteroaryl, and C₆₋₁₀ aryl is optionally substituted with 1, 2, or 3 substituents independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino.

Embodiment 42 is the method of Embodiment 41, wherein the compound of Formula (I) has formula:

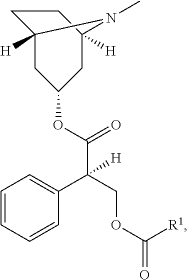

or a pharmaceutically acceptable salt thereof.

Embodiment 43 is the method of Embodiment 42, wherein R¹ is selected from C₁₋₃₄ alkyl, C₂₋₃₄ alkenyl, and C₂₋₃₄ alkynyl.

Embodiment 44 is the method of Embodiment 41, wherein the ophthalmic composition comprises a semifluorinated alkane.

Embodiment 45 is the method of Embodiment 44, wherein the semifluorinated alkane is selected from perfluorohexyloctane (F6H8), perfluorohexylhexane (F6H6), perfluorobutylbutane (F4H4), and perfluorohexylethane (F6H2).

Embodiment 46 is the method of any one of Embodiments 41-45, wherein the compound of Formula (I) is selected from any one of the following compounds:

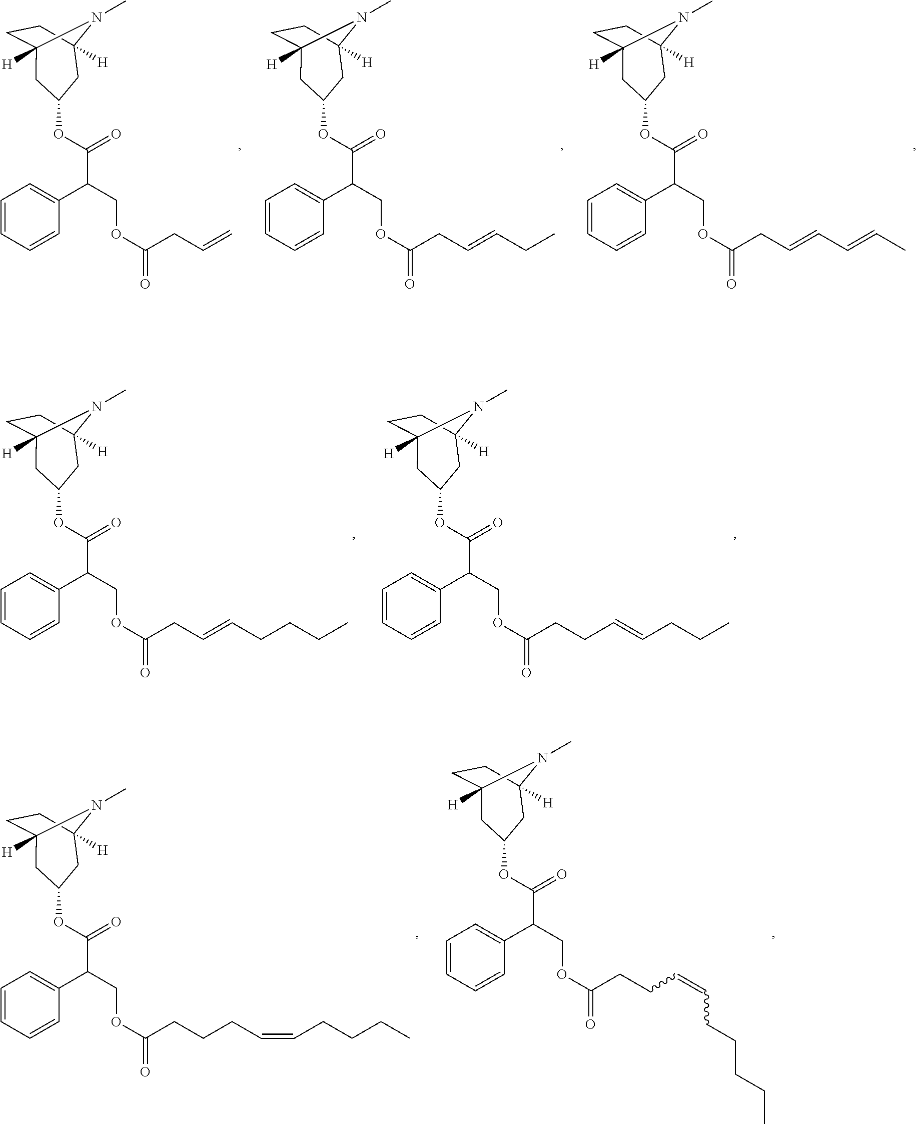

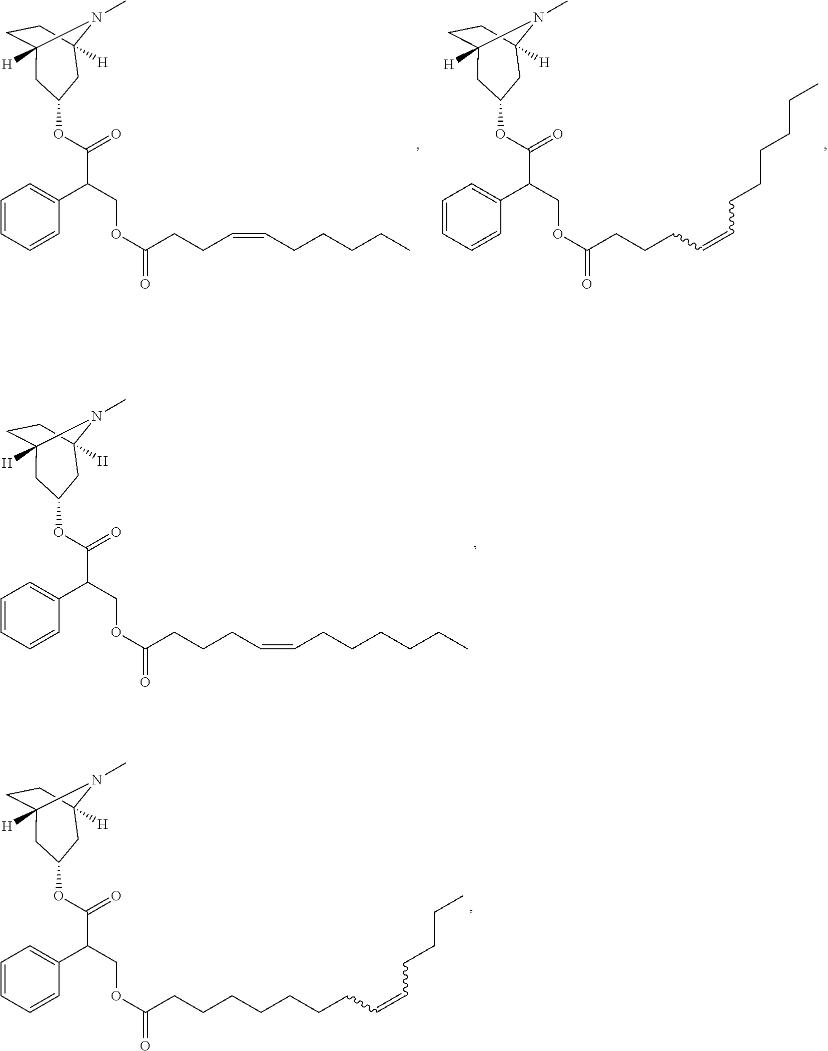

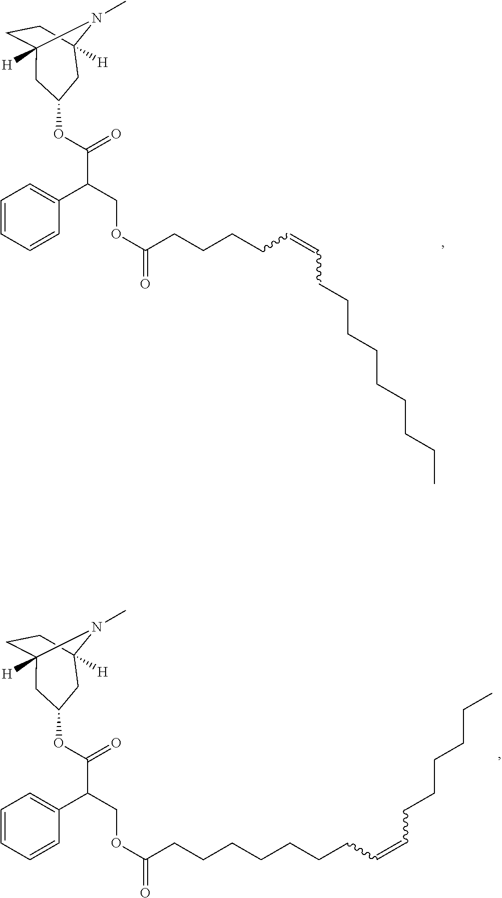 [SMILES: CCCCCCCCCC=CCCCCC(=O)OCC(C(=O)O[C@H]1C[C@H]2CC[C@@H](C1)N2C)c1ccccc1 ; CCCCCCC=CCCCCCCCC(=O)OCC(C(=O)O[C@H]1C[C@H]2CC[C@@H](C1)N2C)c1ccccc1]

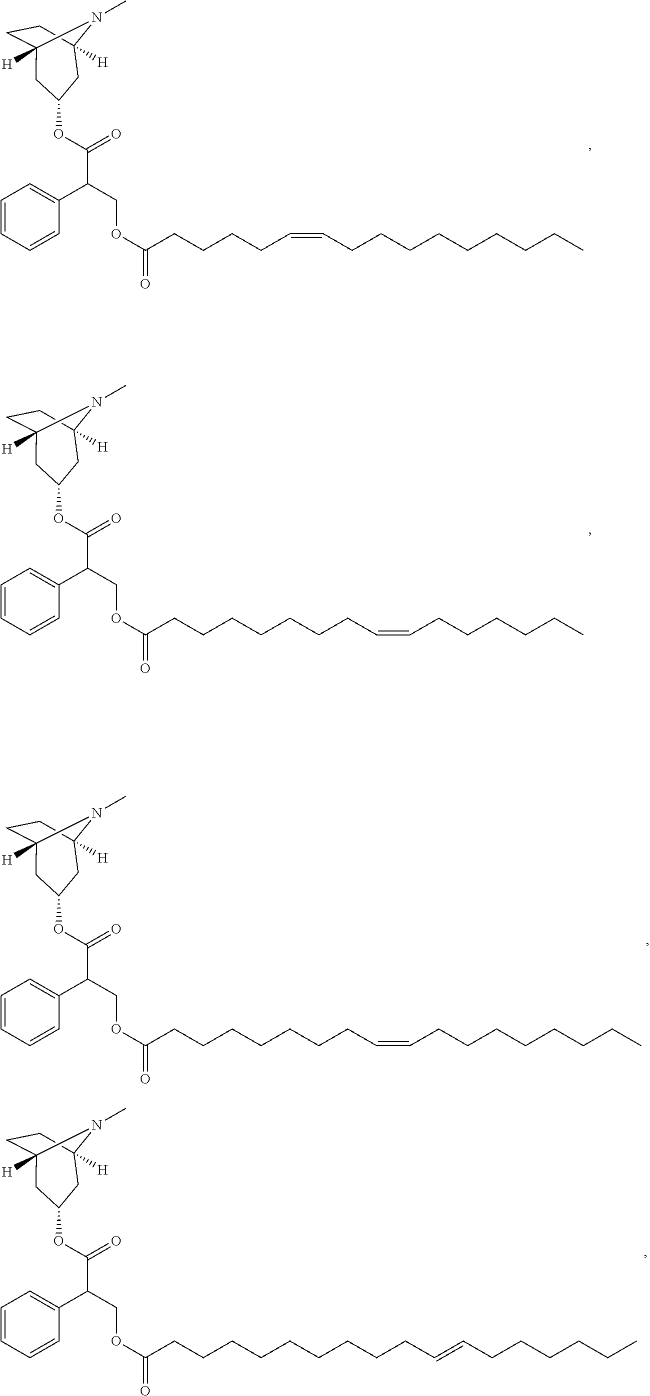

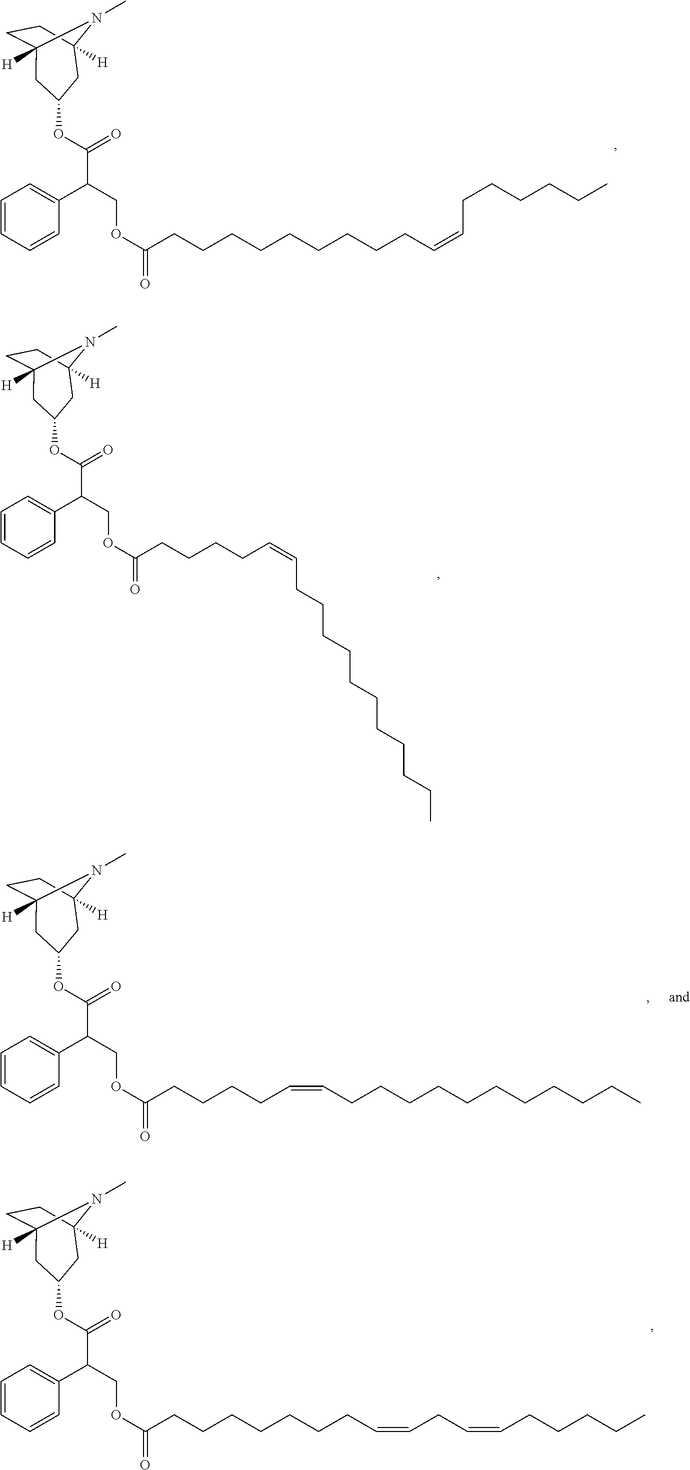 [SMILES: CCCCCC/C=C\CCCCCCCCCC(=O)OCC(C(=O)O[C@H]1C[C@H]2CC[C@@H](C1)N2C)c1ccccc1 ; CCCCCCCCCCC/C=C\CCCCC(=O)OCC(C(=O)O[C@H]1C[C@H]2CC[C@@H](C1)N2C)c1ccccc1 ; CCCCCCCCCCC/C=C\CCCCC(=O)OCC(C(=O)O[C@H]1C[C@H]2CC[C@@H](C1)N2C)c1ccccc1 ; CCCCC/C=C\C/C=C\CCCCCCCC(=O)OCC(C(=O)O[C@H]1C[C@H]2CC[C@@H](C1)N2C)c1ccccc1]

or a pharmaceutically acceptable salt thereof.

Embodiment 47 is the method of any one of Embodiments 41-45, wherein the compound of Formula (I) is selected from any one of the following compounds:

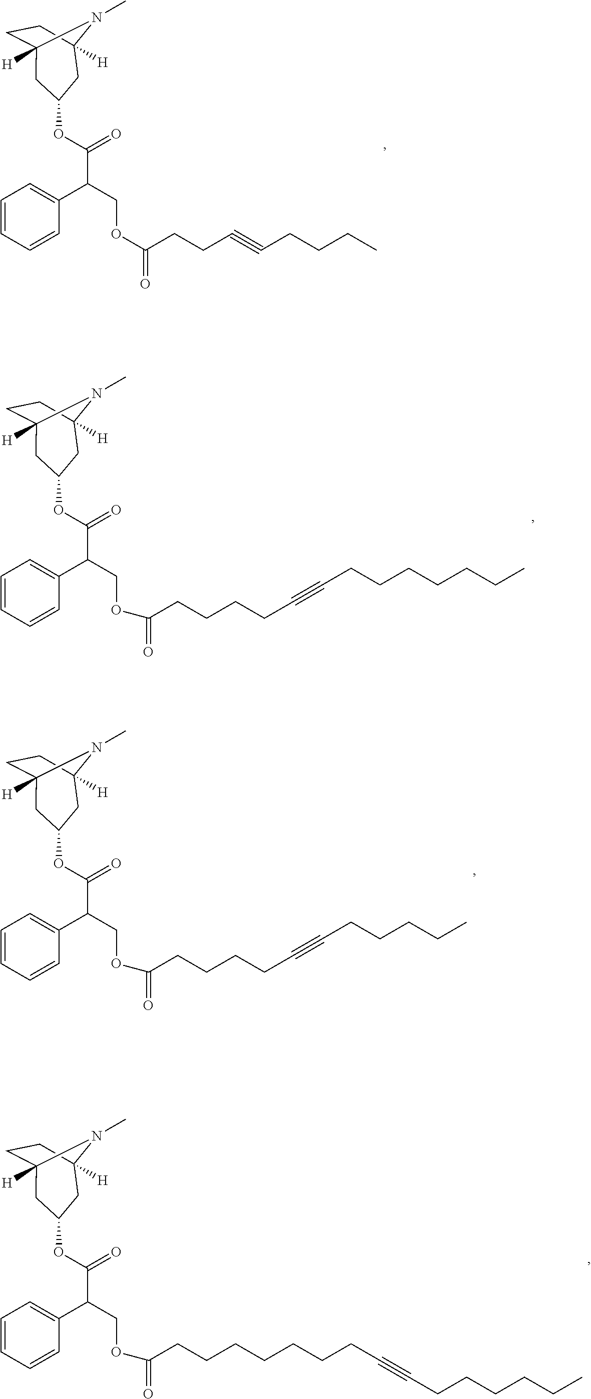 [SMILES: CCCCC#CCCC(=O)OCC(C(=O)O[C@H]1C[C@H]2CC[C@@H](C1)N2C)c1ccccc1 ; CCCCCCCC#CCCCCC(=O)OCC(C(=O)O[C@H]1C[C@H]2CC[C@@H](C1)N2C)c1ccccc1 ; CCCCCC#CCCCCC(=O)OCC(C(=O)O[C@H]1C[C@H]2CC[C@@H](C1)N2C)c1ccccc1 ; CCCCCCC#CCCCCCCCC(=O)OCC(C(=O)O[C@H]1C[C@H]2CC[C@@H](C1)N2C)c1ccccc1]

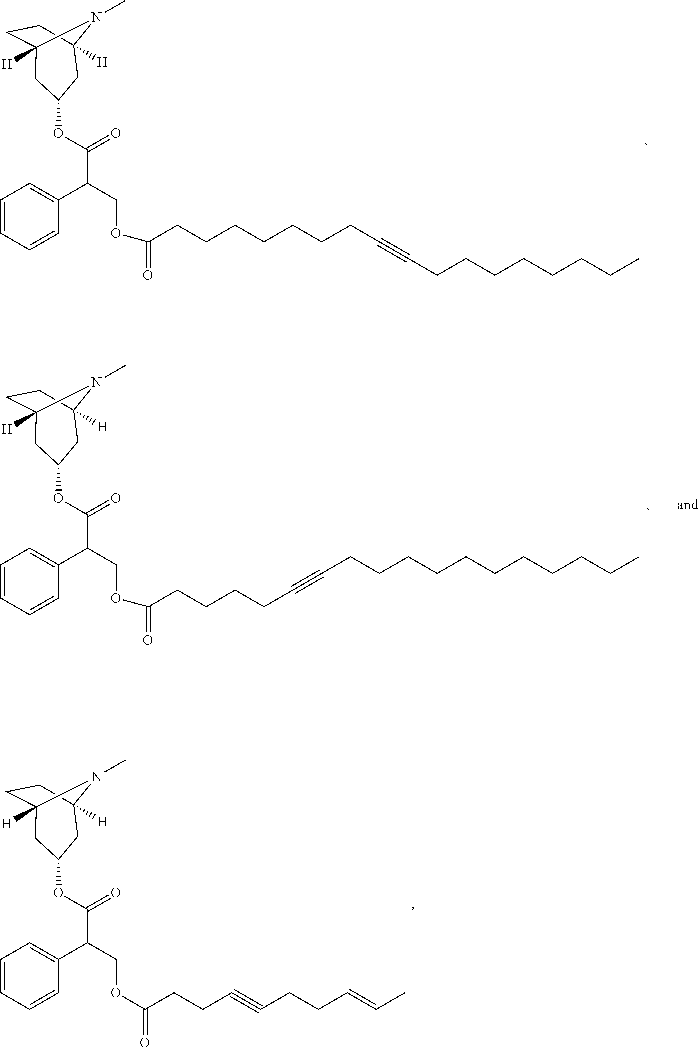 [SMILES: CCCCCCCCC#CCCCCCCCC(=O)OCC(C(=O)O[C@H]1C[C@H]2CC[C@@H](C1)N2C)c1ccccc1 ; CCCCCCCCCCCC#CCCCCC(=O)OCC(C(=O)O[C@H]1C[C@H]2CC[C@@H](C1)N2C)c1ccccc1 ; C/C=C/CCC#CCCC(=O)OCC(C(=O)O[C@H]1C[C@H]2CC[C@@H](C1)N2C)c1ccccc1]

or a pharmaceutically acceptable salt thereof.

Embodiment 48 is the method of any one of Embodiments 41-45, wherein the compound of Formula (I) is selected from any one of the following compounds:

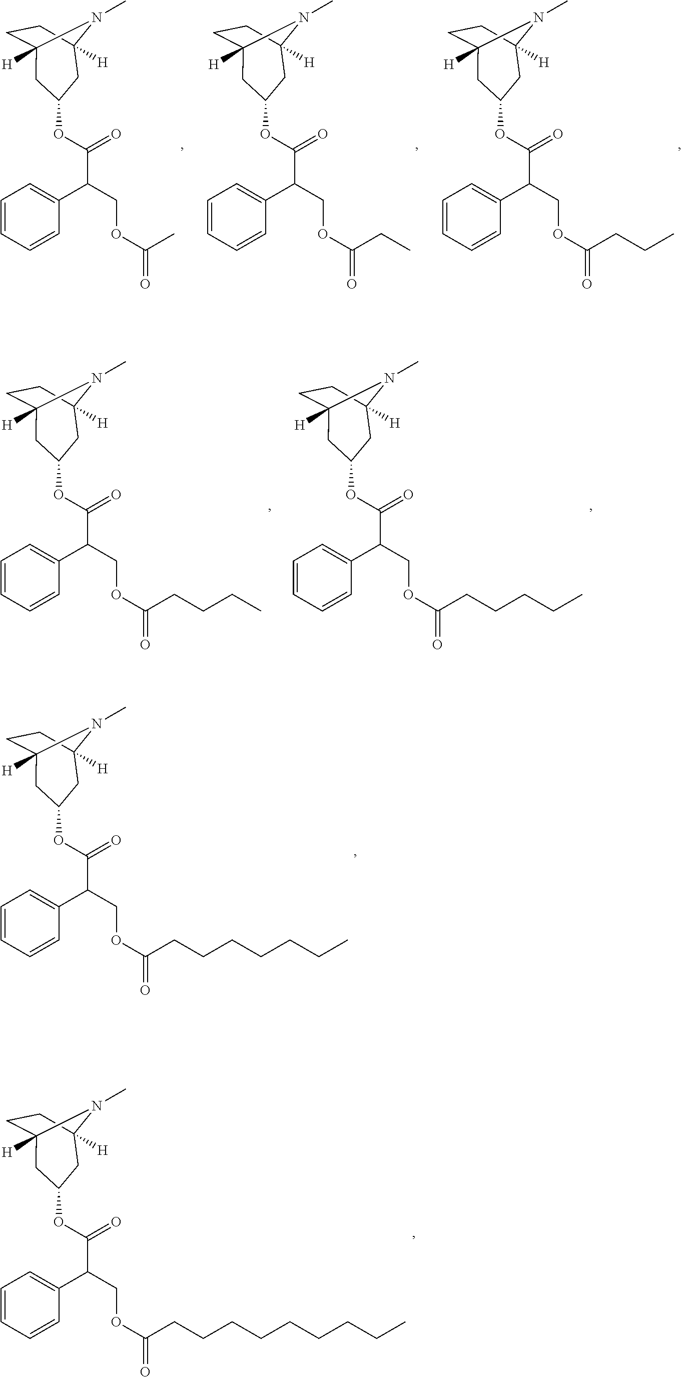

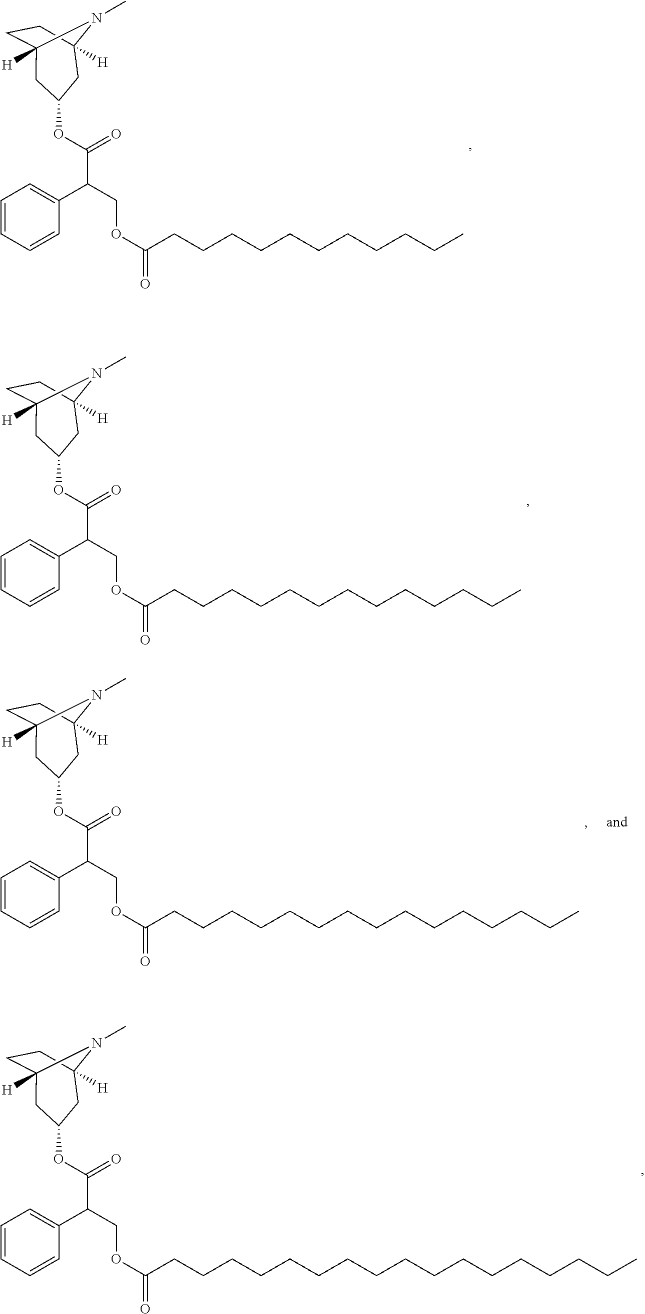

or a pharmaceutically acceptable salt thereof.

Embodiment 49 is a pharmaceutical composition comprising:
i) a compound of Formula (I):

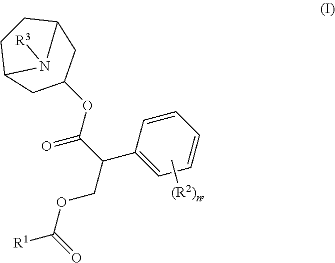

or a pharmaceutically acceptable salt thereof, wherein:
R³ is selected from H, C₁₋₆ alkyl, and C₁₋₆ haloalkyl;
each R² is independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino;
n is selected from 0, 1, 2, 3, 4, and 5; and
R¹ is selected from C₇₋₃₄ alkyl, C₇₋₃₄ alkenyl, C₇₋₃₄ alkynyl, C₃₋₁₀ cycloalkyl, 4-10 membered heterocycloalkyl, 5-14 membered heteroaryl, and C₆₋₁₀ aryl, wherein said C₇₋₃₄ alkyl, C₇₋₃₄ alkenyl, and C₇₋₃₄ alkynyl are each optionally substituted with 1 or 2 substituents independently selected from C₃₋₁₀ cycloalkyl, 4-10 membered heterocycloalkyl, 5-14 membered heteroaryl, and C₆₋₁₀ aryl, and each of said C₃₋₁₀ cycloalkyl, 4-10 membered heterocycloalkyl, 5-14 membered heteroaryl, and C₆₋₁₀ aryl is optionally substituted with 1, 2, or 3 substituents independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino; and
ii) a pharmaceutically acceptable carrier comprising a semifluorinated alkane.

Embodiment 50 is the pharmaceutical composition of Embodiment 49, wherein the semifluorinated alkane is selected from perfluorohexyloctane (F6H8), perfluorohexylhexane (F6H6), perfluorobutylbutane (F4H4), and perfluorohexylethane (F6H2).

Embodiment 51 the pharmaceutical composition of Embodiment 49, wherein R¹ is selected from C₇₋₂₁ alkyl, C₇₋₂₁ alkenyl, and C₇₋₂₁ alkynyl.

Embodiment 52 is the pharmaceutical composition of Embodiment 49, wherein the R¹ is selected from C₇₋₂₁ alkenyl and C₇₋₂₁ alkynyl.

Embodiment 53 is the pharmaceutical composition of Embodiment 49, wherein the compound of Formula (I) is selected from any one of the following compounds:

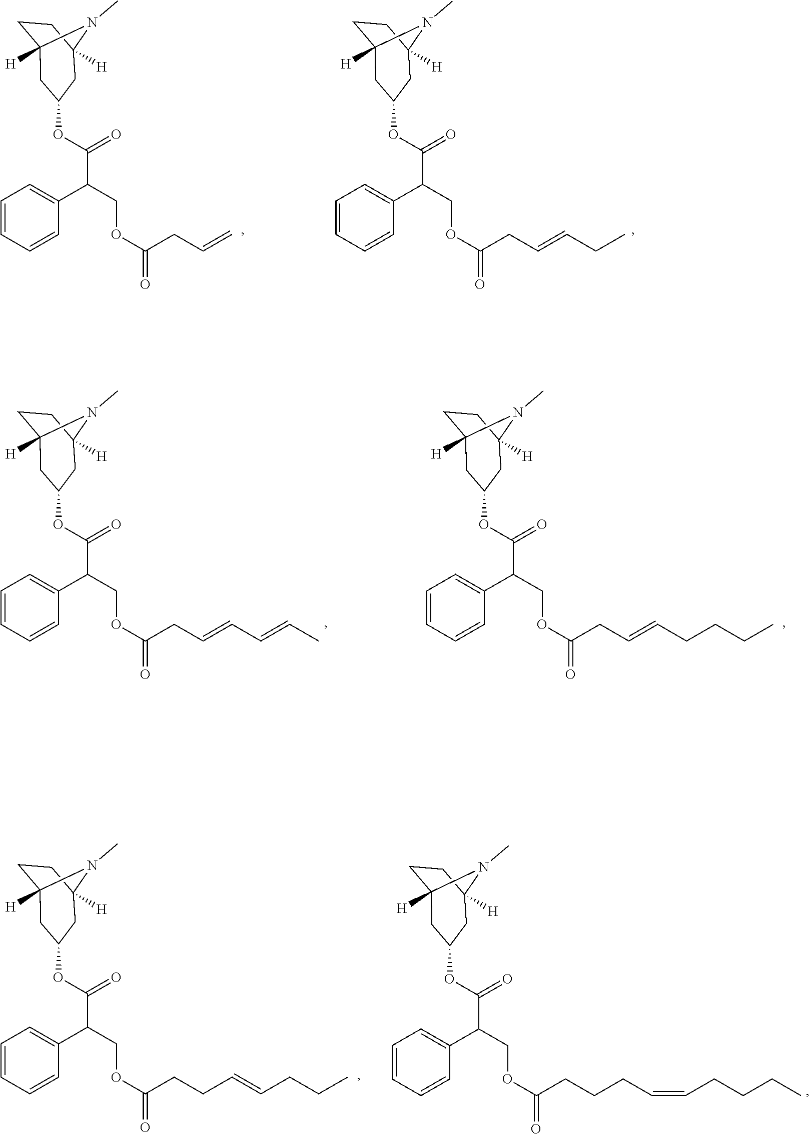

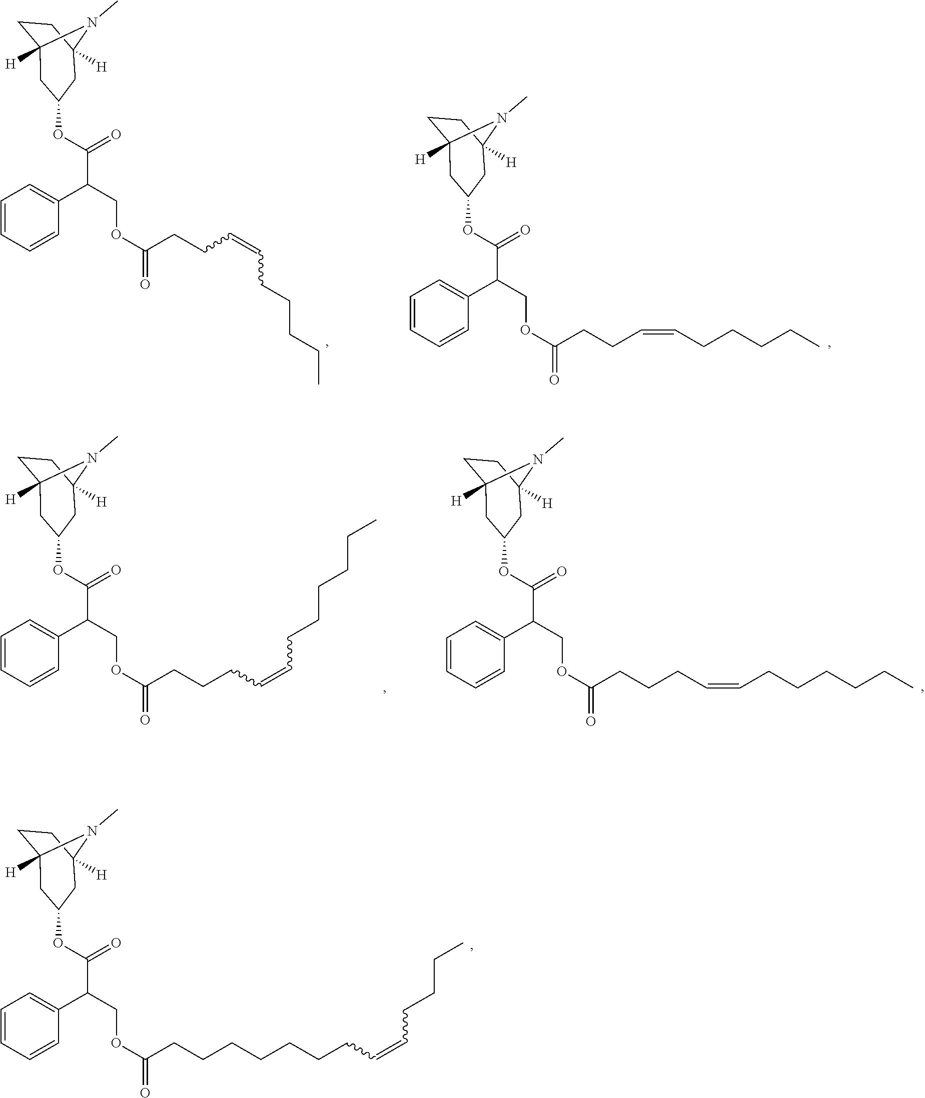 [SMILES: CCCCCC=CCCC(=O)OCC(C(=O)O[C@H]1C[C@H]2CC[C@@H](C1)N2C)c1ccccc1 ; CCCCC/C=C\CCC(=O)OCC(C(=O)O[C@H]1C[C@H]2CC[C@@H](C1)N2C)c1ccccc1 ; CCCCCCC=CCCCC(=O)OCC(C(=O)O[C@H]1C[C@H]2CC[C@@H](C1)N2C)c1ccccc1 ; CCCCCC/C=C\CCCC(=O)OCC(C(=O)O[C@H]1C[C@H]2CC[C@@H](C1)N2C)c1ccccc1 ; CCCCC=CCCCCCCCC(=O)OCC(C(=O)O[C@H]1C[C@H]2CC[C@@H](C1)N2C)c1ccccc1]

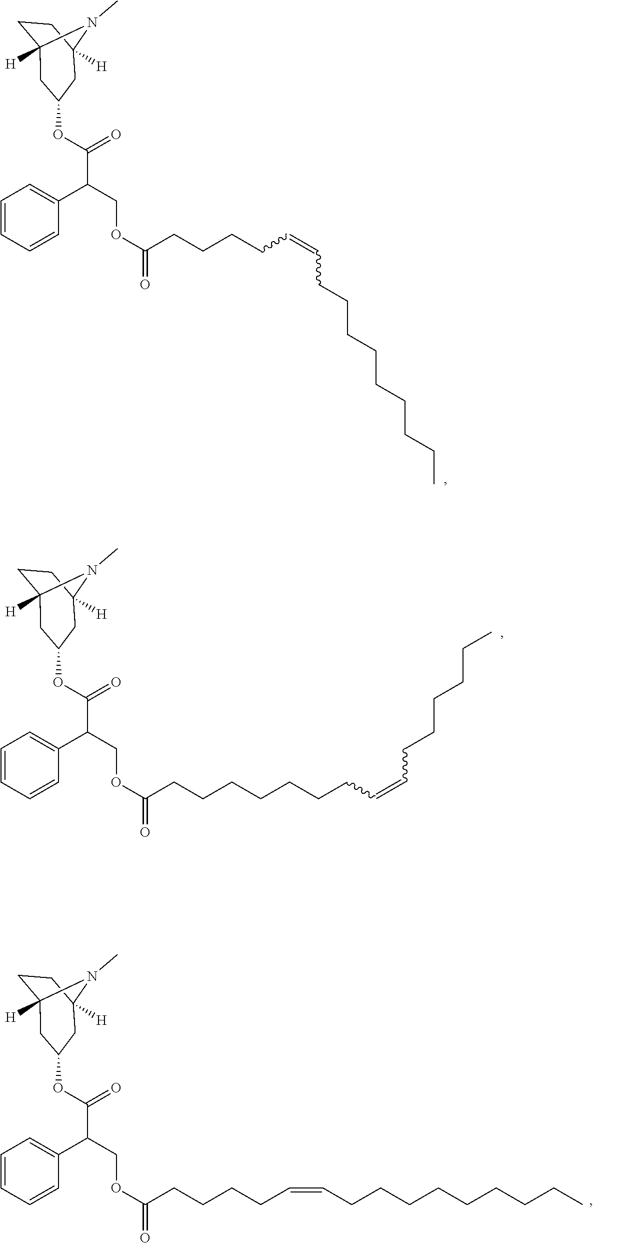 [SMILES: CCCCCCCCCC=CCCCCC(=O)OCC(C(=O)O[C@H]1C[C@H]2CC[C@@H](C1)N2C)c1ccccc1 ; CCCCCCC=CCCCCCCCC(=O)OCC(C(=O)O[C@H]1C[C@H]2CC[C@@H](C1)N2C)c1ccccc1 ; CCCCCCCCC/C=C\CCCCC(=O)OCC(C(=O)O[C@H]1C[C@H]2CC[C@@H](C1)N2C)c1ccccc1]

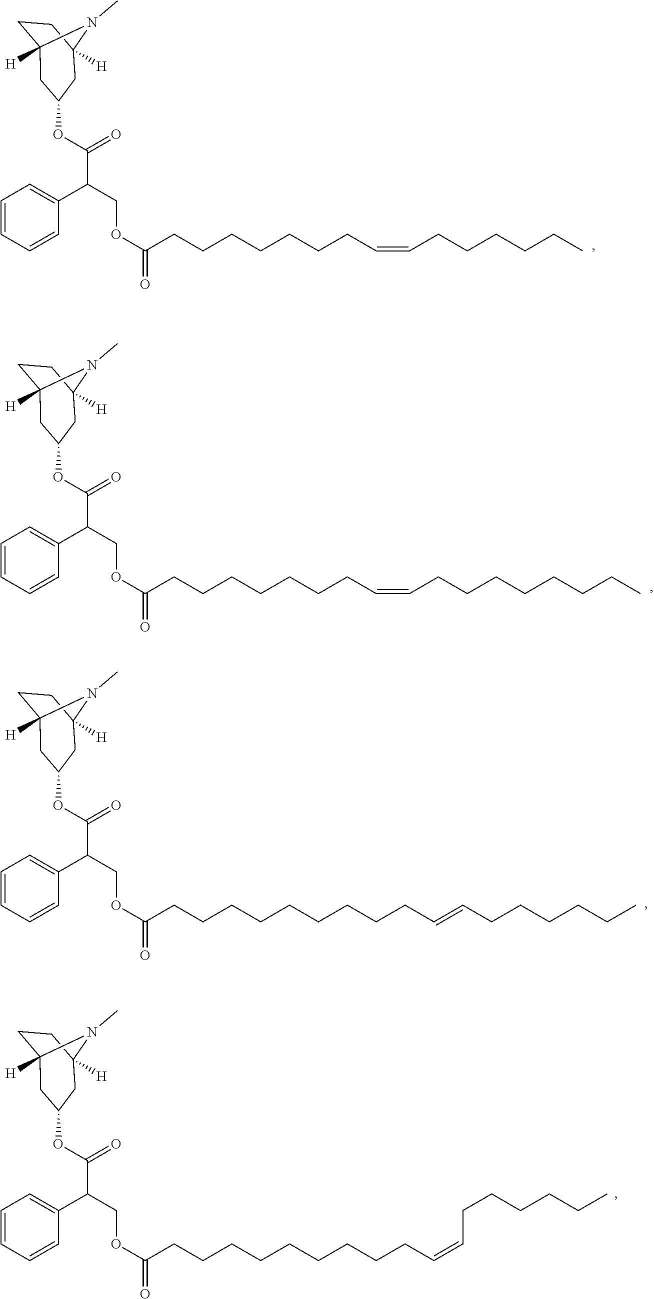 [SMILES: CCCCCC/C=C\CCCCCCCC(=O)OCC(C(=O)O[C@H]1C[C@H]2CC[C@@H](C1)N2C)c1ccccc1 ; CCCCCCCC/C=C\CCCCCCCC(=O)OCC(C(=O)O[C@H]1C[C@H]2CC[C@@H](C1)N2C)c1ccccc1 ; CCCCCC/C=C/CCCCCCCCCC(=O)OCC(C(=O)O[C@H]1C[C@H]2CC[C@@H](C1)N2C)c1ccccc1 ; CCCCCC/C=C\CCCCCCCCCC(=O)OCC(C(=O)O[C@H]1C[C@H]2CC[C@@H](C1)N2C)c1ccccc1]

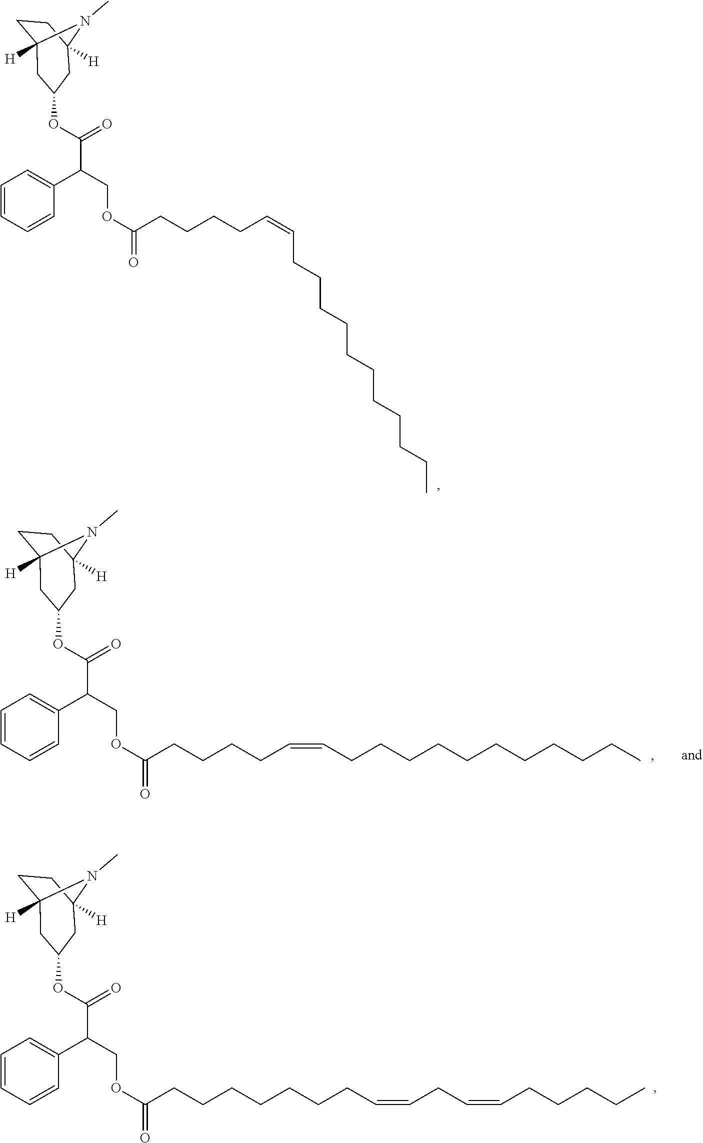 [SMILES: CCCCCCCCCCC/C=C\CCCCC(=O)OCC(C(=O)O[C@H]1C[C@H]2CC[C@@H](C1)N2C)c1ccccc1 ; CCCCCCCCCCC/C=C\CCCCC(=O)OCC(C(=O)O[C@H]1C[C@H]2CC[C@@H](C1)N2C)c1ccccc1 ; CCCCC/C=C\C/C=C\CCCCCCCC(=O)OCC(C(=O)O[C@H]1C[C@H]2CC[C@@H](C1)N2C)c1ccccc1]

or a pharmaceutically acceptable salt thereof.

Embodiment 54 is the pharmaceutical composition of Embodiment 49, wherein the compound of Formula (I) is selected from any one of the following compounds:

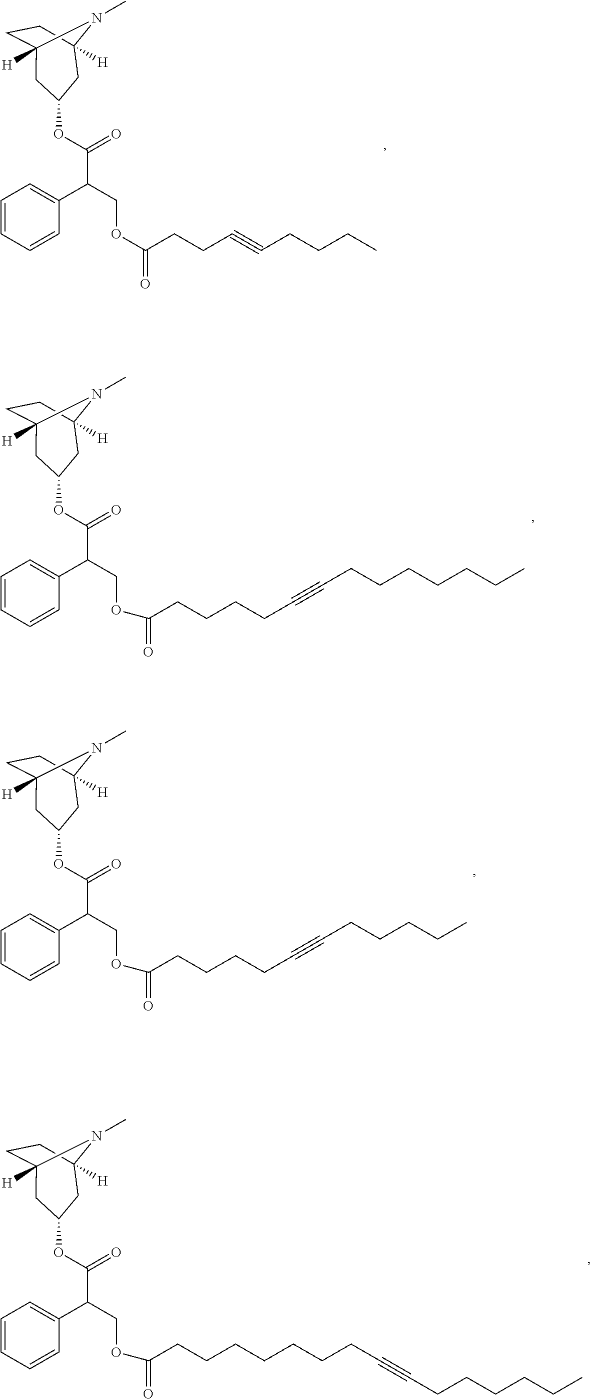 [SMILES: CCCCC#CCCC(=O)OCC(C(=O)O[C@H]1C[C@H]2CC[C@@H](C1)N2C)c1ccccc1 ; CCCCCCCC#CCCCCC(=O)OCC(C(=O)O[C@H]1C[C@H]2CC[C@@H](C1)N2C)c1ccccc1 ; CCCCCC#CCCCCC(=O)OCC(C(=O)O[C@H]1C[C@H]2CC[C@@H](C1)N2C)c1ccccc1 ; CCCCCCC#CCCCCCCCC(=O)OCC(C(=O)O[C@H]1C[C@H]2CC[C@@H](C1)N2C)c1ccccc1]

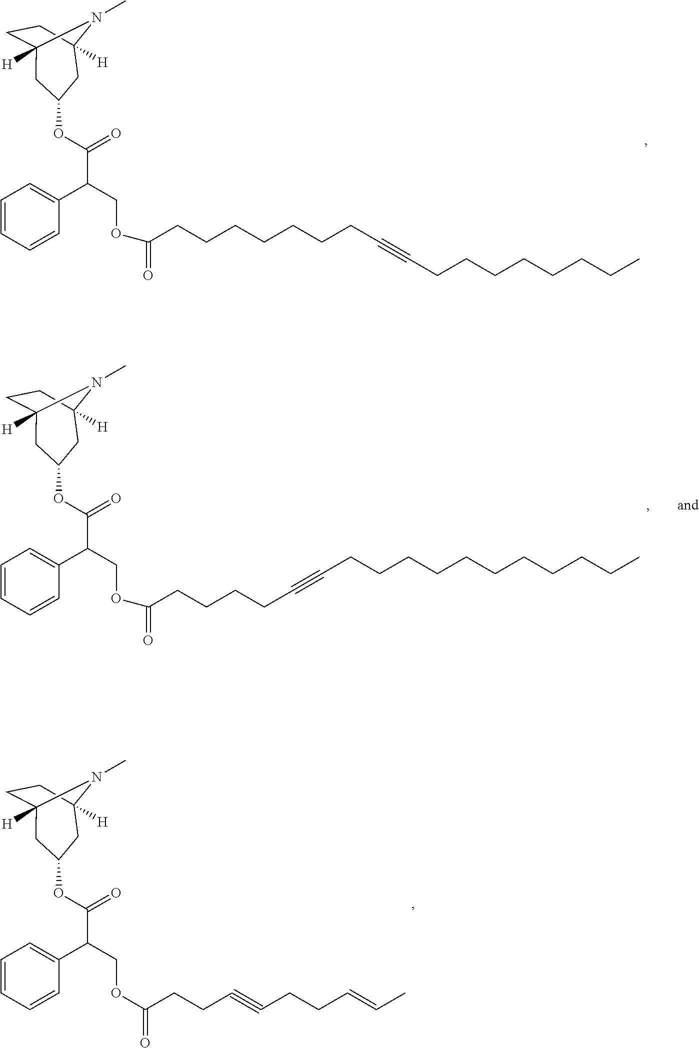 [SMILES: CCCCCCCCC#CCCCCCCCC(=O)OCC(C(=O)O[C@H]1C[C@H]2CC[C@@H](C1)N2C)c1ccccc1 ; CCCCCCCCCCCC#CCCCCC(=O)OCC(C(=O)O[C@H]1C[C@H]2CC[C@@H](C1)N2C)c1ccccc1 ; C/C=C/CCC#CCCC(=O)OCC(C(=O)O[C@H]1C[C@H]2CC[C@@H](C1)N2C)c1ccccc1]

or a pharmaceutically acceptable salt thereof.

Embodiment 55 is the pharmaceutical composition of Embodiment 49, wherein the compound of Formula (I) is selected from any one of the following compounds:

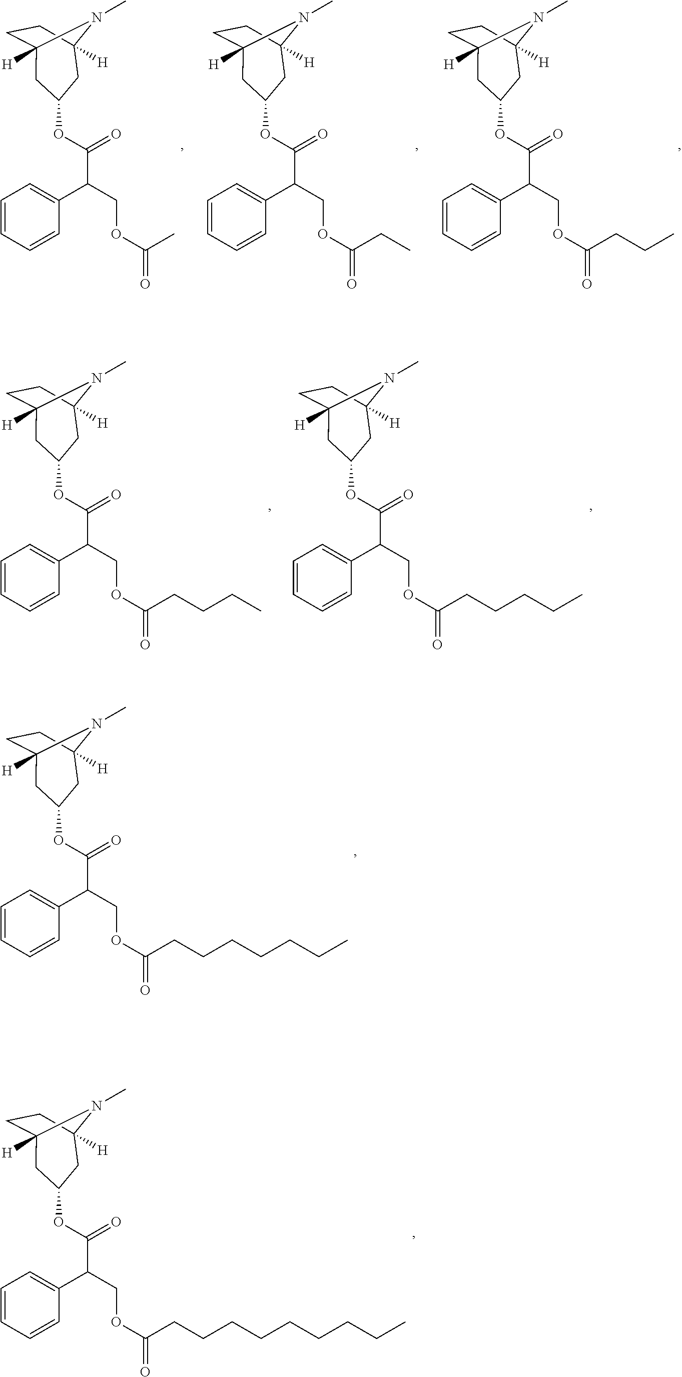

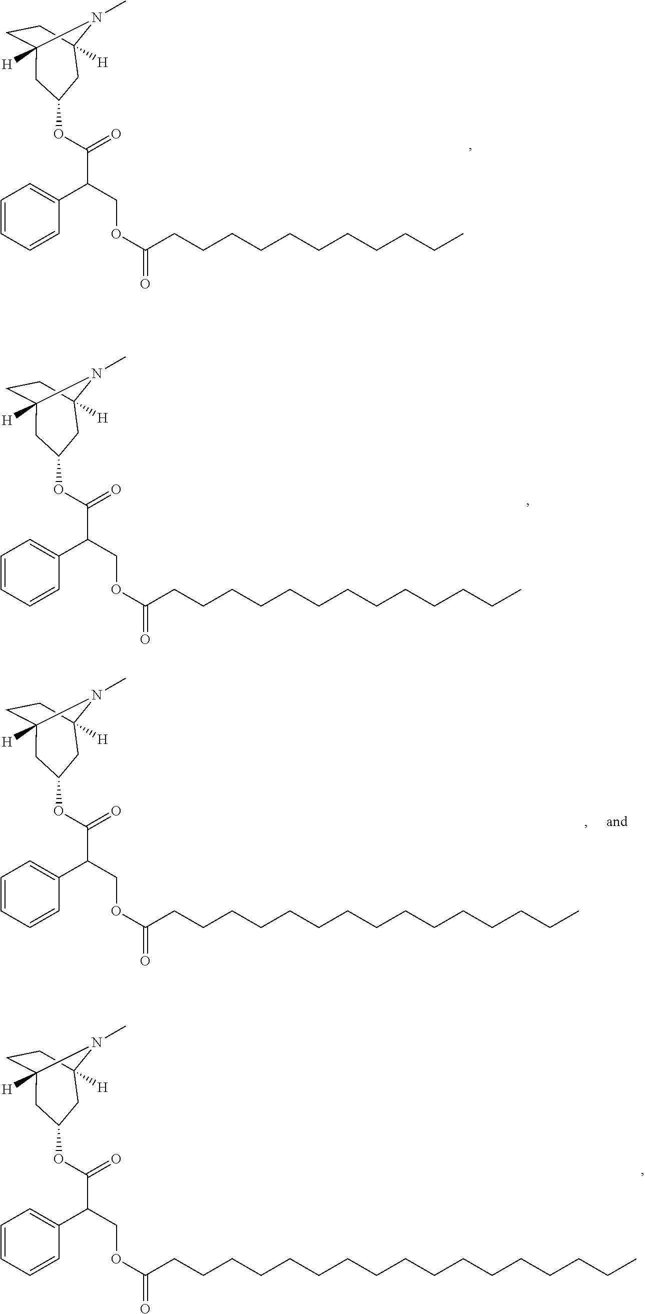

or a pharmaceutically acceptable salt thereof.

Embodiment 56 is a method of treating or preventing a disease or condition where muscarinic acetylcholine receptor is implicated, the method comprising administering to a subject in need thereof a therapeutically effective amount of a pharmaceutical composition of any one of Embodiments 47-53.

Embodiment 57 is the method of Embodiment 56, wherein the pharmaceutical composition is an ophthalmic composition.

### Examples

### General Methods

In general, characterization of the compounds is performed using NMR spectra, which were recorded on 300 and/or 600 MHz Varian and acquired at room temperature. Chemical shifts are given in ppm referenced either to internal TMS or to the solvent signal. All the reagents, solvents, catalysts for which the synthesis is not described are purchased from chemical vendors such as Sigma Aldrich, Fluka, Bio-Blocks, Combi-blocks, TCI, VWR, Lancaster, Oakwood, Trans World Chemical, Alfa, Fisher, Maybridge, Frontier, Matrix, Ukrorgsynth, Toronto, Ryan Scientific, SiliCycle, Anaspec, Syn Chem, Chem-Impex, or MIC-scientific, Ltd; however some known intermediates were or could be prepared according to published procedures. Some compounds of this disclosure can generally be prepared in one step from commercially available literature starting materials. Usually the compounds of the disclosure were purified by column chromatography (Auto-column) on an Teledyne-ISCO CombiFlash with a silica column, unless noted otherwise. The following abbreviations are used throughout the examples:

| | |
|---|---|
| nBu₄NOH | tetrabutylammonium hydroxide |
| DMF | dimethylformamide |
| MPLC | medium-pressure liquid chromatography |
| MeOH | methanol |
| NaCNBH₃ | sodium cyanoborohydride |
| NaOMe | sodium methoxide |
| EtOH | ethanol |
| CDCl₃ | deuterated chloroform |
| NaBH₄ | sodium borohydride |
| MgSO₄ | magnesium sulfate |
| HCl | hydrochloric acid |
| Et₂O | diethyl ether |
| NH₄Cl | ammonium chloride |
| DIBAL—H | diisobutylaluminum hydride |
| K₂CO₃ | potassium carbonate |
| CH₂Cl₂ | dichloromethane |
| CuI | copper iodide |
| NMO | N-methylmorpholine oxide |
| SiO₂ | silica gel, or silica |

### Example 1—Synthesis of 3-(((1R,3r,5S)-8-methyl-8-azabicyclo[3.2.1]octan-3-yl)oxy)-3-oxo-2-phenylpropyl (9Z)-octadec-9-enoate (atropine oleate) (cmpd. 1)

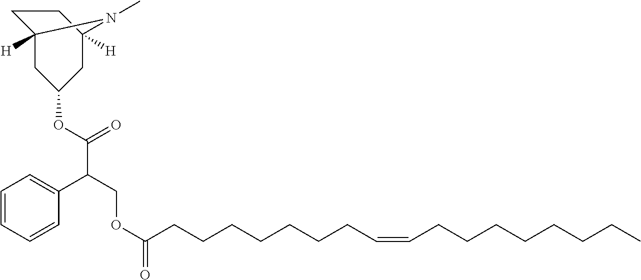 [SMILES: CCCCCCCC/C=C\CCCCCCCC(=O)OCC(C(=O)O[C@H]1C[C@H]2CC[C@@H](C1)N2C)c1ccccc1]

To a solution of (1R,3R,5S)-8-methyl-8-azabicyclo[3.2.1]octan-3-yl 3-hydroxy-2-phenylpropanoate (atropine) (500 mg, 1.29 mmol) in CH₂Cl₂ (10 mL) was added TEA (653 mg, 6.45 mmol) and oleoyl chloride (519 mg, 1.29 mmol). The reaction mixture was stirred at 25° C. for 16 h. The reaction mixture was concentrated in vacuum. The mixture was purified by silica gel column (CH₂Cl₂:MeOH=0 to 10:1) to afford the title compound (120 mg, 28% yield) as a light yellow oil. ¹H NMR (400 MHz, CDCl₃) δ 7.40-7.26 (m, 5H), 5.34 (tt, J=11.2, 5.6 Hz, 2H), 5.14 (t, J=4.2 Hz, 1H), 4.62 (dd, J=11.0, 9.0 Hz, 1H), 4.39 (dd, J=11.0, 6.0 Hz, 1H), 3.92 (dd, J=8.8, 6.0 Hz, 1H), 3.62 (s, 1H), 3.48 (d, J=7.2 Hz, 1H), 2.91 (d, J=52.4 Hz, 2H), 2.63 (s, 3H), 2.27 (dd, J=14.8, 7.4 Hz, 2H), 2.13-1.96 (m, 6H), 1.93 (d, J=16.2 Hz, 1H), 1.85 (s, 1H), 1.75 (d, J=16.0 Hz, 1H), 1.60-1.50 (m, 2H), 1.45-1.16 (m, 22H), 0.88 (t, J=6.8 Hz, 3H). LCMS (ESI) calcd for C₃₅H₅₅NO₄ [M+H]⁺ m/z 554.41, found 554.5.

### Example 2—Synthesis of 3-(((1R,3r,5S)-8-methyl-8-azabicyclo[3.2.1]octan-3-yl)oxy)-3-oxo-2-phenylpropyl (9Z,12Z)-octadeca-9,12-dienoate (atropine linoleate) (cmpd. 2)

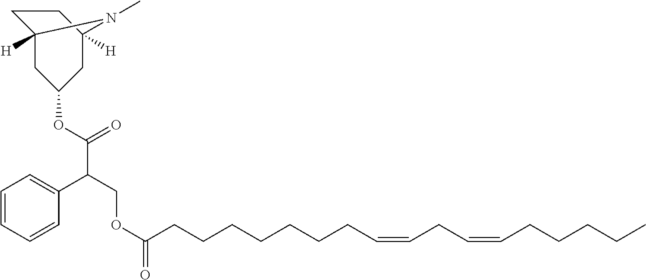 [SMILES: CCCCC/C=C\C/C=C\CCCCCCCC(=O)OCC(C(=O)O[C@H]1C[C@H]2CC[C@@H](C1)N2C)c1ccccc1]

To a solution of atropine (1 g, 0.0035 mol) in CH₂Cl₂ (10 mL) was added 4-DMAP (1.28 g, 0.0105 mol), EDCI (0.34 g, 0.0017 mol) and (9Z,12Z)-octadeca-9,12-dienoic acid (linoleic acid) (1.47 g, 0.0052 mol). The reaction mixture was stirred at 25° C. for 2 h. LCMS showed the reaction worked well. The reaction mixture was poured into water (50 mL) and extracted with EtOAc (3×50 mL). The combined organic layers were dried over anhydrous Na₂SO₄ and concentrated in vacuum. The residue was purified by prep-HPLC (ACN/H₂O (0.1% FA)) to afford the title compound (118 mg, 6% yield) as a yellow solid. Chromatographic columns: Xbridge-C18 150×19 mm, 5 μm. Mobile Phase: ACN/H₂O (0.1% FA), gradient: 30/70. LCMS (ESI) calcd. for C₃₅H₅₃NO₄ [M+H]⁺ m/z 552.4, found 552.4. ¹H NMR (400 MHz, CDCl₃) δ 7.40-7.26 (m, 5H), 5.42-5.26 (m, 4H), 5.10 (s, 1H), 4.62 (dd, J=11.2, 9.2 Hz, 1H), 4.39 (dd, J=11.2, 6.0 Hz, 1H), 3.92 (dd, J=8.8, 6.0 Hz, 1H), 2.77 (t, J=6.4 Hz, 2H), 2.49 (s, 2H), 2.26 (t, J=7.6 Hz, 2H), 2.03 (dt, J=12.4, 5.6 Hz, 6H), 1.85 (d, J=15.6 Hz, 3H), 1.67 (d, J=16.0 Hz, 3H), 1.61-1.49 (m, 3H), 1.43-1.20 (m, 15H), 0.89 (t, J=6.8 Hz, 3H).

### Example 3—Fatty Acid Esters of Atropine

The following compounds 3 (atropine caprate), 4 (atropine caprylate), 5 (atropine laurate), 6 (atropine myristate), 7 (atropine palmitate), 8 (atropine (Z)-dec-5-enoate), 9 (atropine octadec-11-enoate), 10 (atropine (Z)-octadec-6-enoate), 11 (atropine (E)-oct-3-enoate), 12 (atropine octadec-6-ynoate), 13 (atropine dec-4-enoate), 14 (atropine dodec-5-enoate), 15 (atropine hexadec-6-enoate), 16 (atropine hexadec-9-enoate), and 17 (atropine tetradec-9-enoate) were prepared from atropine and the corresponding fatty acids according to the methods and procedures described in examples 1 and 2.

| Cmpd. No. | Chemical structure and IUPAC name |
|---|---|
| 3 | 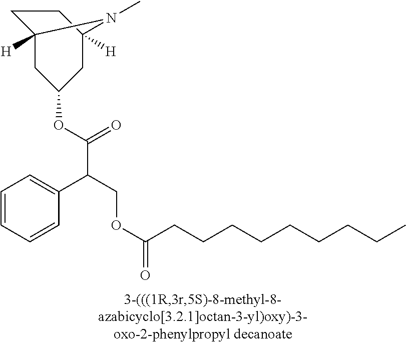 [SMILES: CCCCCCCCCC(=O)OCC(C(=O)O[C@H]1C[C@H]2CC[C@@H](C1)N2C)c1ccccc1] |
| 4 | 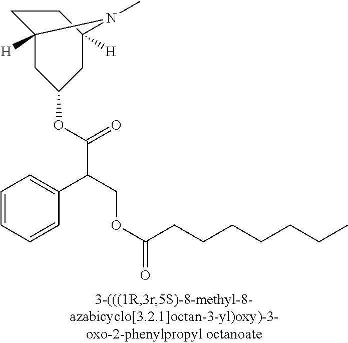 [SMILES: CCCCCCCC(=O)OCC(C(=O)O[C@H]1C[C@H]2CC[C@@H](C1)N2C)c1ccccc1] |
| 5 | 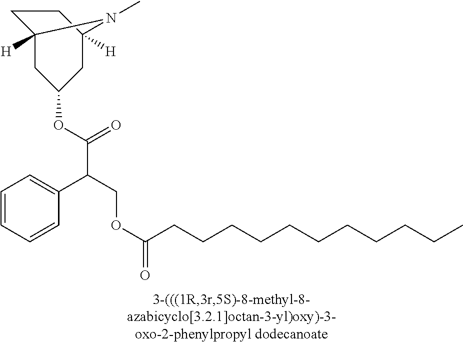 [SMILES: CCCCCCCCCCCC(=O)OCC(C(=O)O[C@H]1C[C@H]2CC[C@@H](C1)N2C)c1ccccc1] |
| 6 | 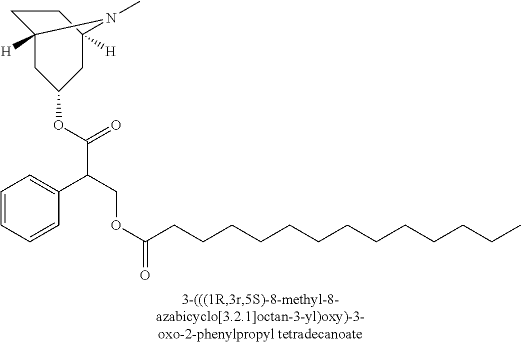 [SMILES: CCCCCCCCCCCCCC(=O)OCC(C(=O)O[C@H]1C[C@H]2CC[C@@H](C1)N2C)c1ccccc1] |
| 7 | 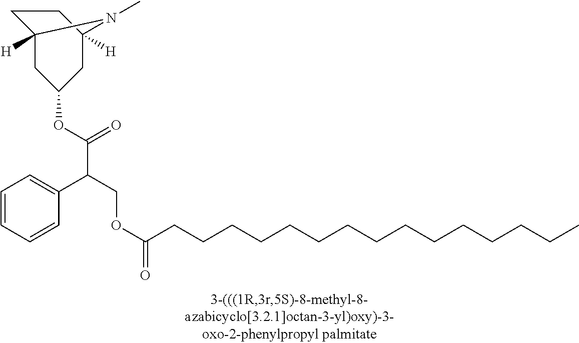 [SMILES: CCCCCCCCCCCCCCCC(=O)OCC(C(=O)O[C@H]1C[C@H]2CC[C@@H](C1)N2C)c1ccccc1] |
| 8 | 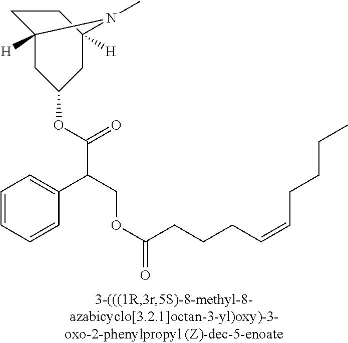 [SMILES: CCCC/C=C\CCCC(=O)OCC(C(=O)O[C@H]1C[C@H]2CC[C@@H](C1)N2C)c1ccccc1] |
| 9 | 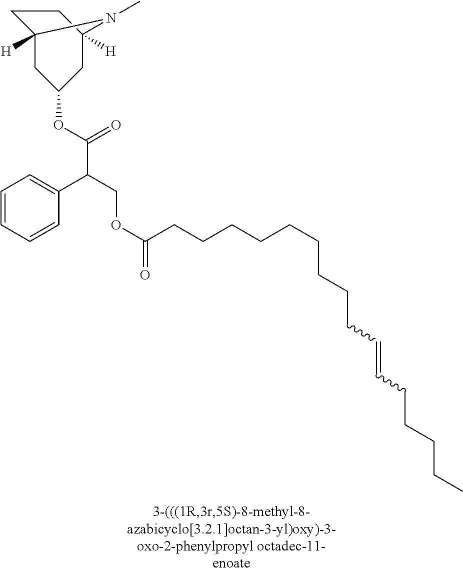 [SMILES: CCCCCC=CCCCCCCCCCC(=O)OCC(C(=O)O[C@H]1C[C@H]2CC[C@@H](C1)N2C)c1ccccc1] |
| 10 | 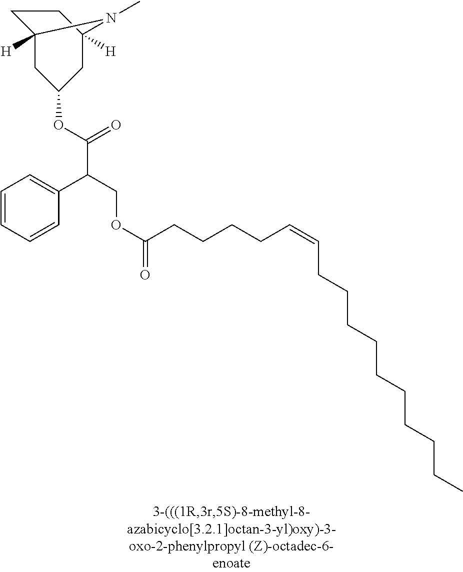 [SMILES: CCCCCCCCCC/C=C\CCCCC(=O)OCC(C(=O)O[C@H]1C[C@H]2CC[C@@H](C1)N2C)c1ccccc1] |
| 11 | 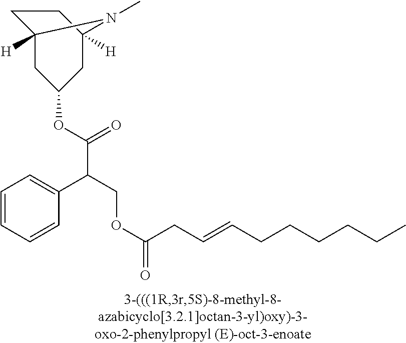 [SMILES: CCCCCC/C=C/CC(=O)OCC(C(=O)O[C@H]1C[C@H]2CC[C@@H](C1)N2C)c1ccccc1] |
| 12 | 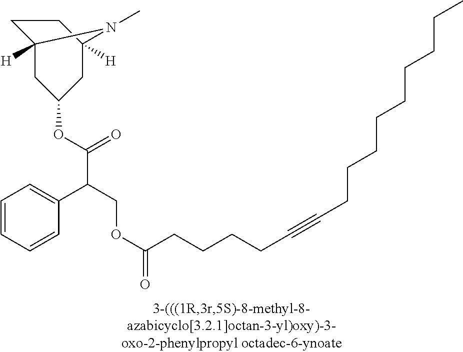 [SMILES: CCCCCCCCCC#CCCCCC(=O)OCC(C(=O)O[C@H]1C[C@H]2CC[C@@H](C1)N2C)c1ccccc1] |
| 13 | 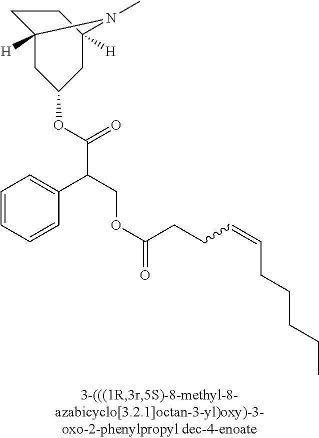 [SMILES: CCCCCC=CCCC(=O)OCC(C(=O)O[C@H]1C[C@H]2CC[C@@H](C1)N2C)c1ccccc1] |
| 14 | 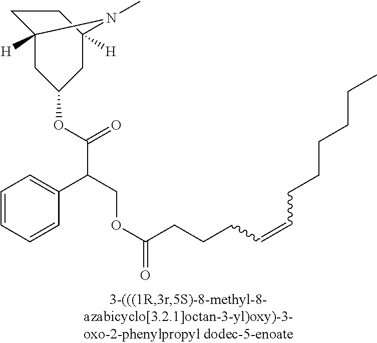 [SMILES: CCCCCCC=CCCCC(=O)OCC(C(=O)O[C@H]1C[C@H]2CC[C@@H](C1)N2C)c1ccccc1] |
| 15 | 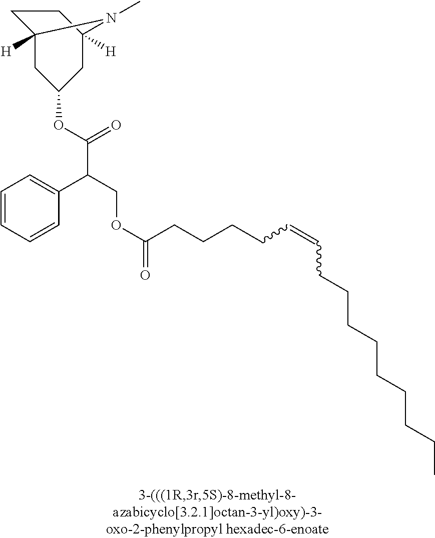 [SMILES: CCCCCCCCCC=CCCCCC(=O)OCC(C(=O)O[C@H]1C[C@H]2CC[C@@H](C1)N2C)c1ccccc1] |
| 16 | 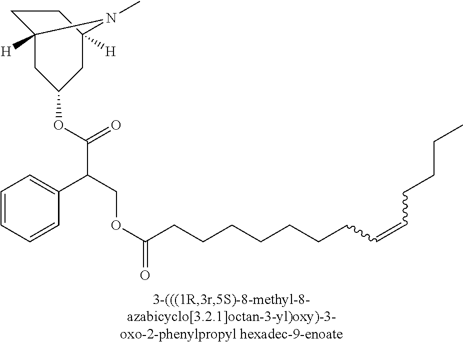 [SMILES: CCCCC=CCCCCCCCC(=O)OCC(C(=O)O[C@H]1C[C@H]2CC[C@@H](C1)N2C)c1ccccc1] |
| 17 | 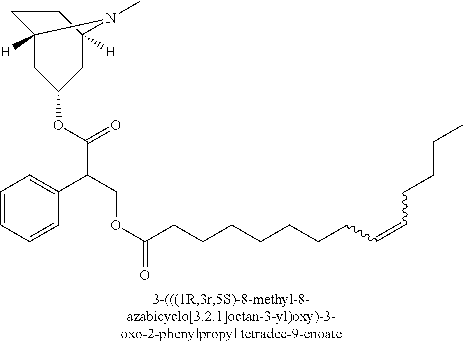 [SMILES: CCCCC=CCCCCCCCC(=O)OCC(C(=O)O[C@H]1C[C@H]2CC[C@@H](C1)N2C)c1ccccc1] |

### Example 4—Dissolution of Atropine Prodrugs in F6H8

Solubility of saturated and unsaturated fatty acid ester prodrugs of atropine (compounds 1-7 as described in examples 1-3) was tested in perfluorohexyloctane (F6H8).

### Preparing HPLC Samples

About 5 mg atropine prodrug compound was weighed into 2 mL centrifuge tube, followed by addition of 1 ml perfluorohexyloctane (F6H8). The mixture was ultrasoned for 3 min and then shaken for 10 min, which was repeated three times. The mixture was then allowed to stand for 30 min, at which time the sample preparation was completed. 100 μL of the mixture were placed in a different centrifuge tube, followed by addition of 0.5 mL acetonitrile. The resultant mixture was shaken for 3 min, then centrifuged for 3 min. The supernatant was taken, followed by addition of 0.5 mL acetonitrile into the centrifuge tube, shaken for 3 min and then centrifuged for 3 min. The formulations above were centrifuged and the supernatants were filtered through 0.45 micron filters without further dilution. Each sample was prepared for HPLC analysis.

### Analyzing the HPLC Samples

The samples were analyzed using a Shim-pack GIST C18, 5 μm HPLC column (250 mm×4.6 mm I.D., 5 μm) and a gradient elution from 100% water to 0.05% trifluoroacetic acid in 100% acetonitrile at a flow rate of 1.1 ml/min for 25 minutes. The chromatograms were monitored at UV at 214 nm. The retention time of the tested compounds were recorded as the following:

| Compd. | HPLC retention time (min.) |
|---|---|
| 1 | 14.3 |
| 2 | 14.3 |
| 7 | 14.5 |
| 9 | 14.7 |
| 13 | 14.1 |
| 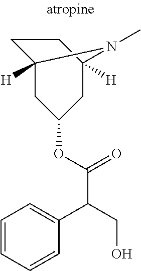 [SMILES: CN1[C@@H]2CC[C@H]1C[C@@H](OC(=O)C(CO)c1ccccc1)C2] | 11.5 |
| 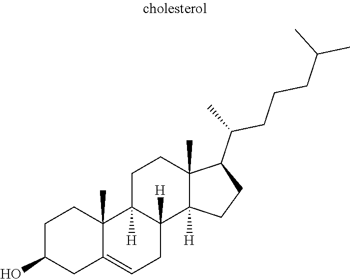 | 13.8 |
| 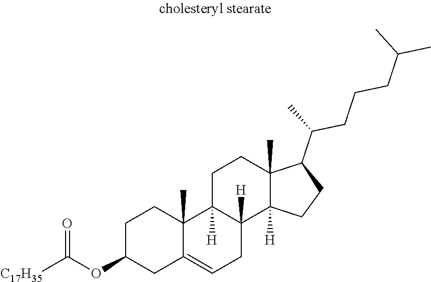 | 16.8 |

### Results of Solubility Test

The solubility of various atropine prodrugs, as well as cholesterol and cholesteryl stearate in F6H8 was determined by HPLC and the results are shown in the Table below:

| compound | solubility (μg/mL) |
|---|---|
| 1 | 5000 |
| 2 | 3870 |
| 7 | 5610 |
| 13 | 4900 |
| 9 | 16180 |
| atropine | 129 |
| cholesterol | 79.65 |
| cholesteryl stearate | 26.23 |

Discussion of Solubility Differences Between Atropine, Cholesterol, and their Esters

The solubility of cholesterol in F6H8 was measured as 79.65 μg/mL and the solubility of cholesteryl stearate, the saturated fatty acid ester of cholesterol, in F6H8, was measured as 26.23 μg/mL. Therefore, both cholesterol and cholesteryl stearate dissolved poorly in F6H8 and the stearate fatty acid esterification of cholesterol did not improve the solubility in F6H8. On the other hand, although the solubility of atropine in F6H8 was poor as measured to be 129 μg/mL, the saturated and unsaturated fatty acid esterification of atropine showed, surprisingly, the marked improvement in the solubility in F6H8.

### Example 5—Formulation of Atropine Oleate in F6H8

The following formulations of atropine prodrugs were prepared in F6H8 matrix. 4 mg of atropine oleate (cmpd. 1) was mixed with 1 mL of perfluorohexyloctane (F6H8), stirred and homogenized for 1 hour to make 0.4% atropine oleate formulation. 0.5 mL of 0.4% atropine oleate formulation was then added with 0.5 mL of perfluorohexyloctane (F6H8) and stirred for 1 hour to make 0.2% atropine oleate formulation. Furthermore, 0.5 mL of 0.2% atropine oleate formulation was added with 0.5 mL of perfluorohexyloctane (F6H8) and stirred for 1 hour to make 0.10% atropine oleate formulation. All three formulations of 0.4%, 0.2% and 0.1% atropine oleate formulations were filtered with 0.2 μm filter to make the final formulations for 0.4%, 0.2% and 0.1% atropine oleate.

### Example 6—Formulation of Atropine Linoleate in F6H8

The following formulations of atropine prodrugs were prepared in F6H8 matrix. 4 mg of atropine linoleate (cmpd. 2) was mixed with 1 mL of perfluorohexyloctane (F6H8), stirred and homogenized for 1 hour to make 0.4% atropine linoleate formulation. Atropine linoleate formulation were filtered with 0.2 μm filter to make the final formulation for 0.4% atropine linoleate.

### Example 7—In Vivo Pharmacology Study in Dutch Belted Rabbits

Three female Dutch Belted rabbits were randomly assigned to each group selected from vehicle, positive control of 0.01% atropine sulfate monohydrate in normal saline, 0.4% atropine oleate, and 0.4% atropine linoleate by Provantis or Excel based on body weight. The test compound was administered to each animal by topical ocular instillation of 40 μL of eye drop formulation just once at day 1. The pupil size of both eyes of all animals were measured at baseline (30 minutes before dosing), 0.5 hour, 1 hour, 2 hour, 4 hour, 6 hour, 8 hour, 12 hour, and 24 hour after dosing. Pupil sizes were measured in both eyes of all animals on 3 separate days during acclimation prior to dosing initiation. Set the light intensity in the procedure room so that the animal's pupil size was about 6-7 mm. Light intensity of the procedure room for pupil size measurement should be maintained constant throughout the pupil size measurement procedures in the study. Pupil size change from baseline was calculated for each eye of each individual animal separately for each postdose timepoint. Mean and standard error of pupil size change from baseline were calculated for each group per postdose timepoint and plotted mean of pupil change from baseline against timepoint comparisons.

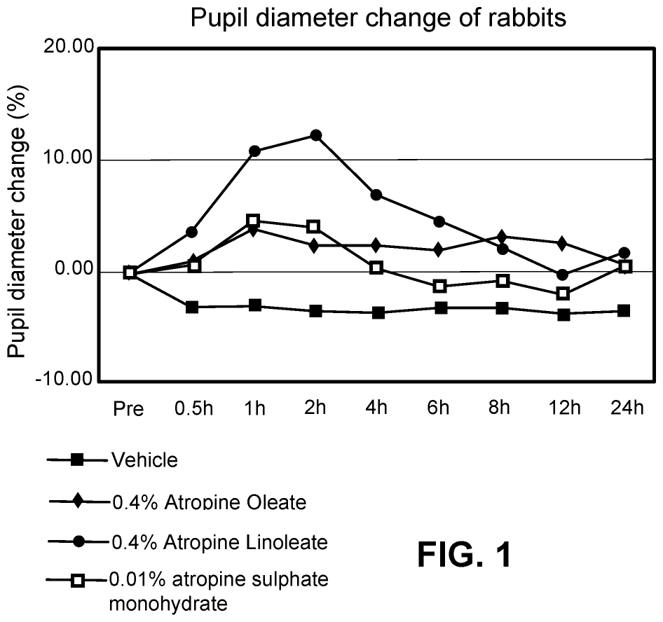

The experimental result was summarized in FIG. 1. Pupil size was dilated at about 5% from baseline at 1 hour or 2 hour post-dose for 0.01% atropine sulfate monohydrate as a positive control while pupil size remained largely unchanged from baseline for vehicle as a negative control. 0.4% atropine oleate in F6H8 formulation showed comparable pupil size dilation as the 0.01% atropine sulfate monohydrate, the positive control, and retained the pupil dilation throughout the measurement period. Meanwhile, 0.4% atropine linoleate showed over 10% change from baseline on the pupil size diameter change and remained significantly dilated from baseline during the measurement period. FIG. 1: pupil size changes from baseline in rabbits after a single eye drop administration of test article formulations, positive and negative controls

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound of Formula (I): 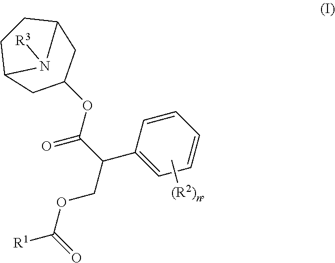
or a pharmaceutically acceptable salt thereof, wherein:
R³ is selected from H, C₁₋₆ alkyl, and C₁₋₆ haloalkyl;
each R² is independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino;
n is selected from 0, 1, 2, 3, 4, and 5; and
R¹ is selected from C₇₋₃₄ alkyl, C₇₋₃₄ alkenyl, C₇₋₃₄ alkynyl, C₃₋₁₀ cycloalkyl, 4-10 membered heterocycloalkyl, 5-14 membered heteroaryl, and C₆₋₁₀ aryl, wherein said C₇₋₃₄ alkyl, C₇₋₃₄ alkenyl, and C₇₋₃₄ alkynyl are each optionally substituted with 1 or 2 substituents independently selected from C₃₋₁₀ cycloalkyl, 4-10 membered heterocycloalkyl, 5-14 membered heteroaryl, and C₆₋₁₀ aryl, and each of said C₃₋₁₀ cycloalkyl, 4-10 membered heterocycloalkyl, 5-14 membered heteroaryl, and C₆-10 aryl is optionally substituted with 1, 2, or 3 substituents independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino;
with a proviso that the compound of Formula (I) is not atropine oleate or hyoscyamine oleate.

2. The compound of claim 1, wherein the compound of Formula (I) has formula: 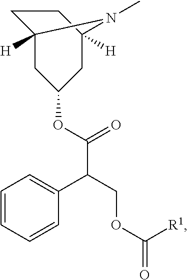
or a pharmaceutically acceptable salt thereof.

3. The compound of claim 1, wherein the compound is selected from any one of the following compounds: 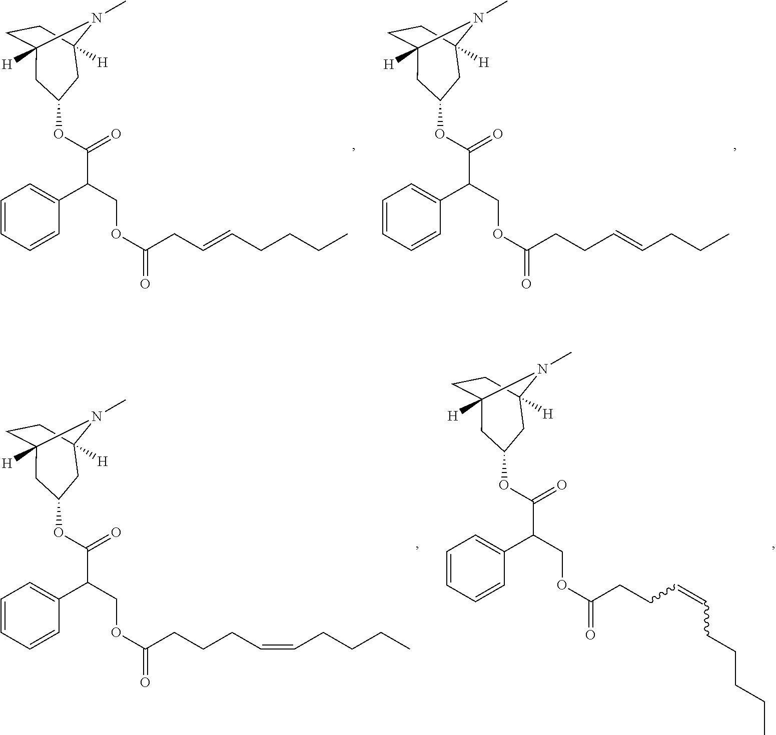 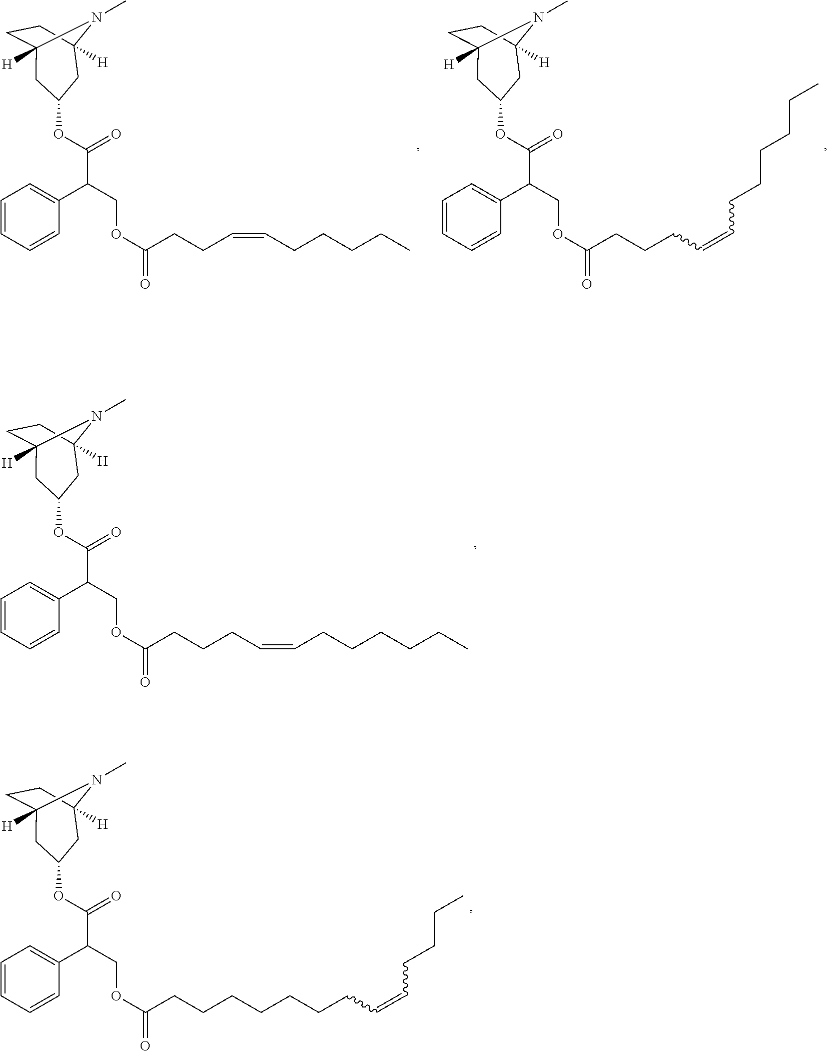 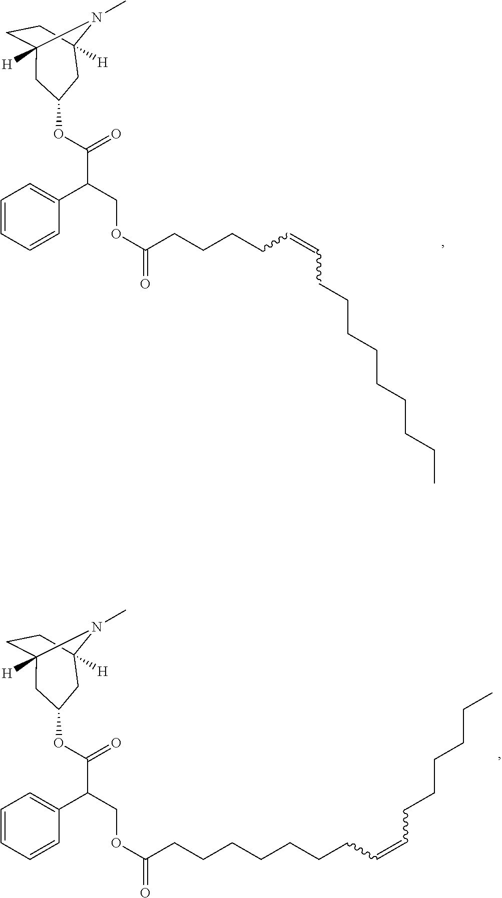 [SMILES: CCCCCCCCCC=CCCCCC(=O)OCC(C(=O)O[C@H]1C[C@H]2CC[C@@H](C1)N2C)c1ccccc1 ; CCCCCCC=CCCCCCCCC(=O)OCC(C(=O)O[C@H]1C[C@H]2CC[C@@H](C1)N2C)c1ccccc1] 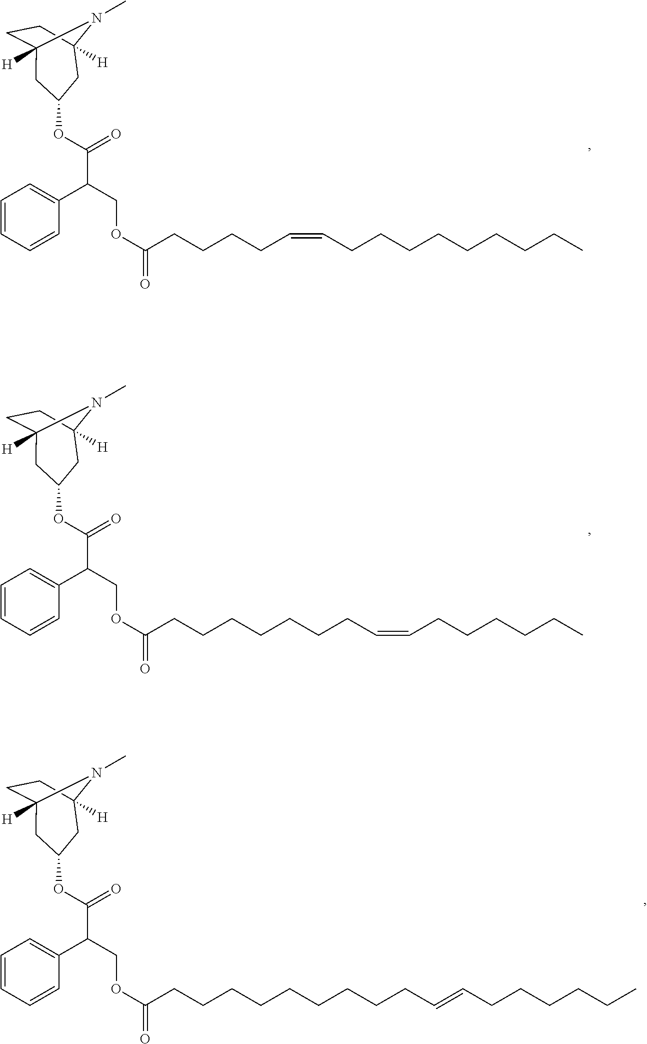 [SMILES: CCCCCCCCC/C=C\CCCCC(=O)OCC(C(=O)O[C@H]1C[C@H]2CC[C@@H](C1)N2C)c1ccccc1 ; CCCCCC/C=C\CCCCCCCC(=O)OCC(C(=O)O[C@H]1C[C@H]2CC[C@@H](C1)N2C)c1ccccc1 ; CCCCCC/C=C/CCCCCCCCCC(=O)OCC(C(=O)O[C@H]1C[C@H]2CC[C@@H](C1)N2C)c1ccccc1] 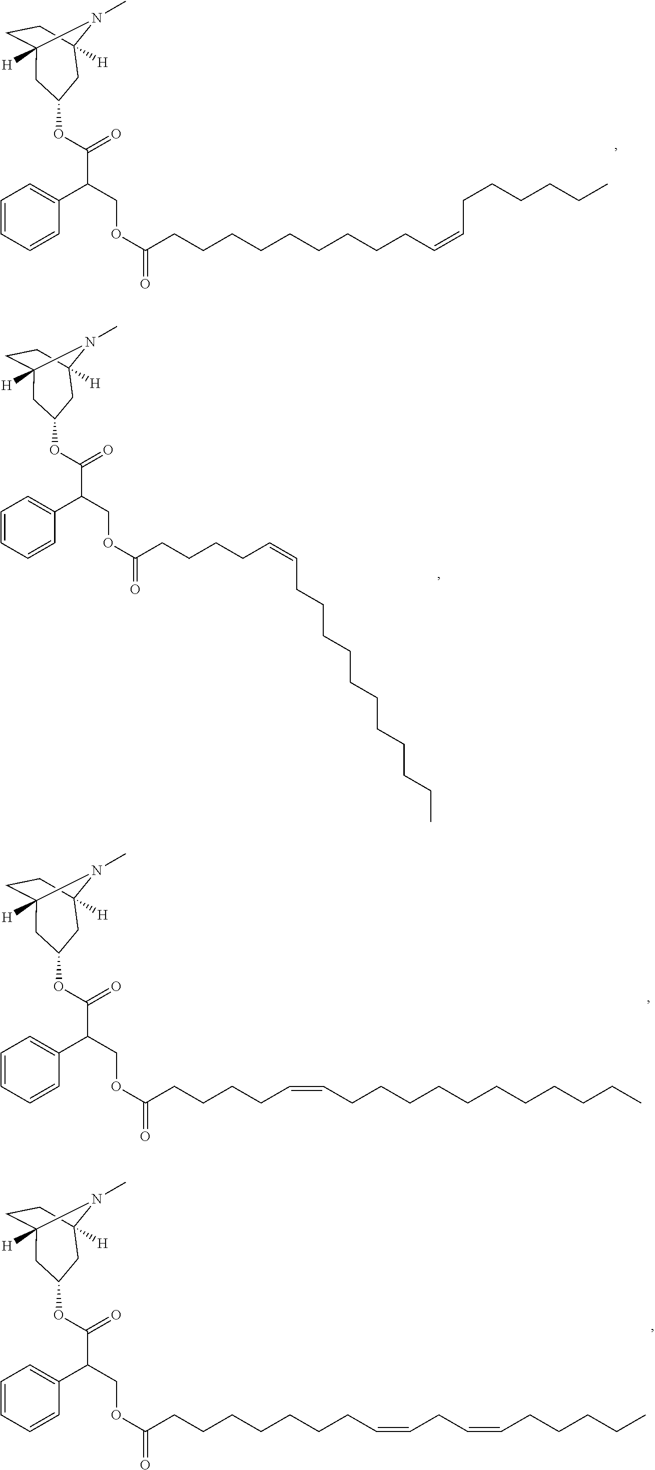 [SMILES: CCCCCC/C=C\CCCCCCCCCC(=O)OCC(C(=O)O[C@H]1C[C@H]2CC[C@@H](C1)N2C)c1ccccc1 ; CCCCCCCCCCC/C=C\CCCCC(=O)OCC(C(=O)O[C@H]1C[C@H]2CC[C@@H](C1)N2C)c1ccccc1 ; CCCCCCCCCCC/C=C\CCCCC(=O)OCC(C(=O)O[C@H]1C[C@H]2CC[C@@H](C1)N2C)c1ccccc1 ; CCCCC/C=C\C/C=C\CCCCCCCC(=O)OCC(C(=O)O[C@H]1C[C@H]2CC[C@@H](C1)N2C)c1ccccc1] 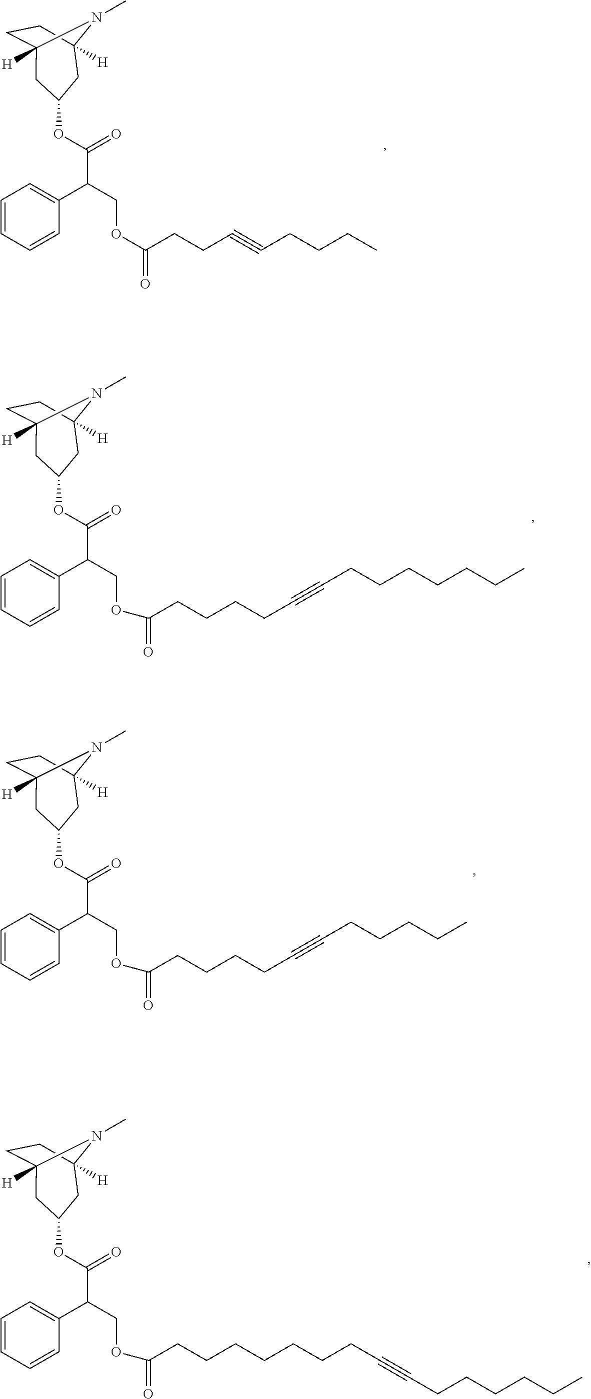 [SMILES: CCCCC#CCCC(=O)OCC(C(=O)O[C@H]1C[C@H]2CC[C@@H](C1)N2C)c1ccccc1 ; CCCCCCCC#CCCCCC(=O)OCC(C(=O)O[C@H]1C[C@H]2CC[C@@H](C1)N2C)c1ccccc1 ; CCCCCC#CCCCCC(=O)OCC(C(=O)O[C@H]1C[C@H]2CC[C@@H](C1)N2C)c1ccccc1 ; CCCCCCC#CCCCCCCCC(=O)OCC(C(=O)O[C@H]1C[C@H]2CC[C@@H](C1)N2C)c1ccccc1] 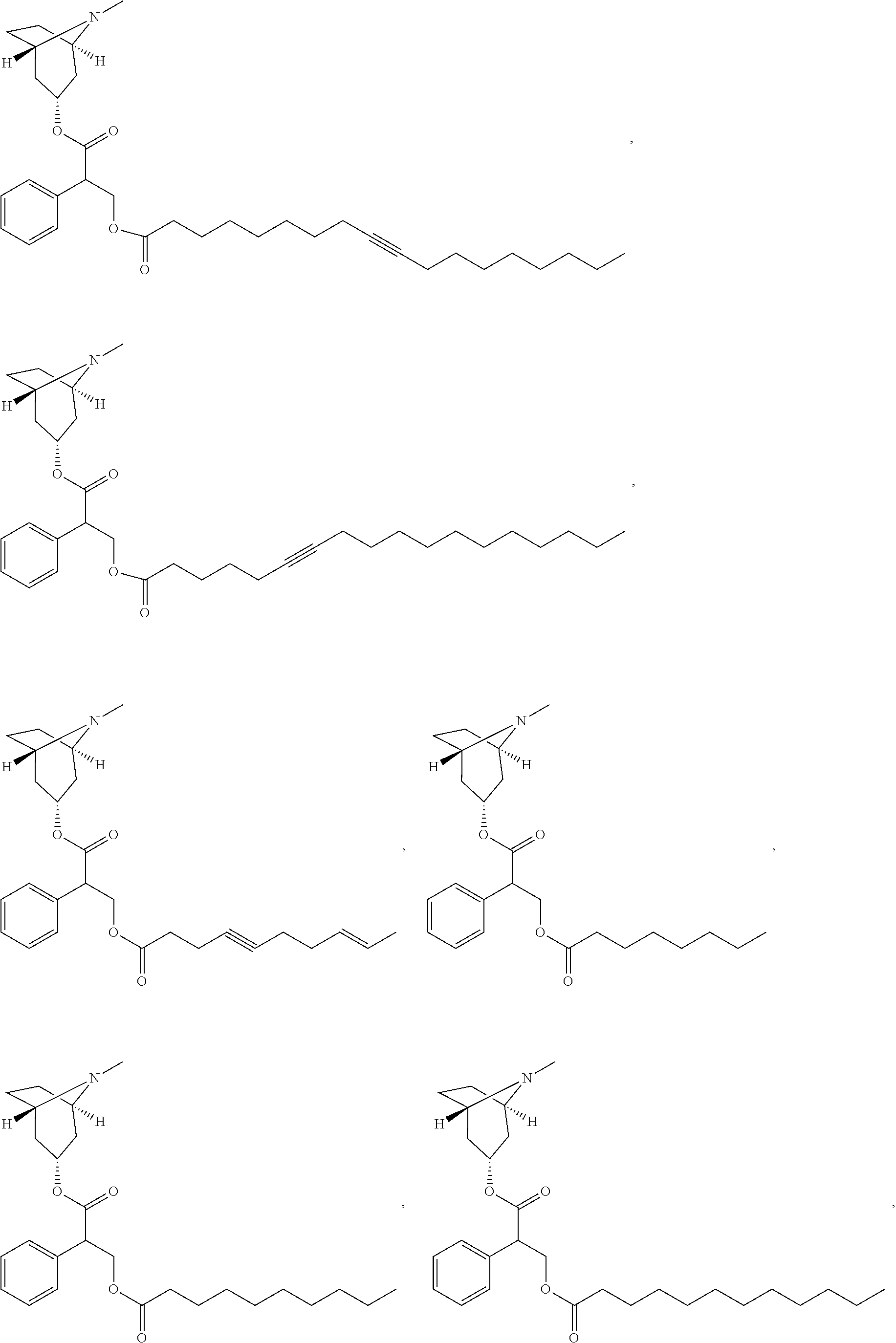 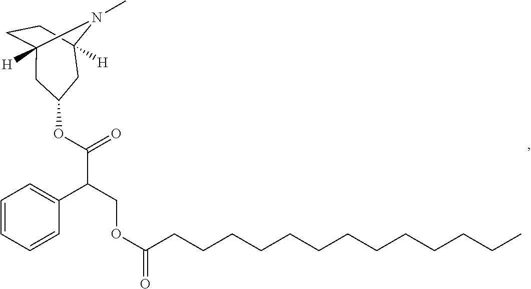 [SMILES: CCCCCCCCCCCCCC(=O)OCC(C(=O)O[C@H]1C[C@H]2CC[C@@H](C1)N2C)c1ccccc1] 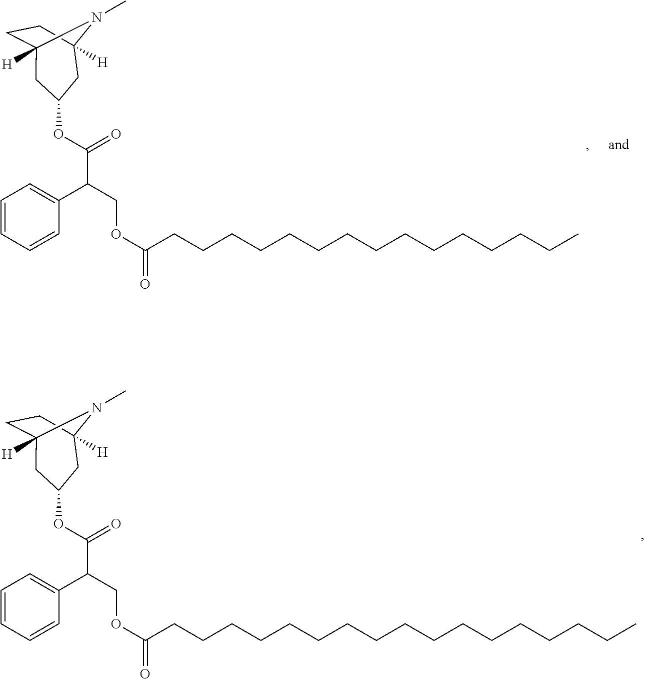 [SMILES: CCCCCCCCCCCCCCCC(=O)OCC(C(=O)O[C@H]1C[C@H]2CC[C@@H](C1)N2C)c1ccccc1 ; CCCCCCCCCCCCCCCCCC(=O)OCC(C(=O)O[C@H]1C[C@H]2CC[C@@H](C1)N2C)c1ccccc1]
or a pharmaceutically acceptable salt thereof.

4. A pharmaceutical composition comprising a compound of claim 1, or a pharmaceutically acceptable salt thereof, and a pharmaceutically acceptable carrier.

5. The pharmaceutical composition of claim 4, wherein the pharmaceutically acceptable carrier comprises a semifluorinated alkane.

6. A method of treating or preventing a disease or condition where muscarinic acetylcholine receptor is implicated, the method comprising administering to a subject in need thereof a therapeutically effective amount of a compound of claim 1, or a pharmaceutically acceptable salt thereof.

7. The method of claim 6, wherein the compound of claim 1, or a pharmaceutically acceptable salt thereof, is used for causing cycloplegic refraction in the eye of the subject, is used for causing mydriasis in the eye of the subject, is used to relieve the eye floater symptoms, is used as a patching therapy for children with amblyopia or lazy eye, or is used to reduce salivation and bronchial secretions.

8. The method of claim 6, wherein the disease or condition is a painful ciliary muscle spasm in the eye, myopia progression, a heart condition, hyperhidrosis, or poisoning.

9. A method for causing a condition selected from cycloplegic refraction and mydriasis in the eye of the subject, for relieving an eye floater symptom in a subject, for patching therapy for children with amblyopia or lazy eye, or for treating or preventing a condition selected from painful ciliary muscle spasm in the eye and a myopia progression, the method comprising administering to an eye of the subject an ophthalmic composition comprising a compound of Formula (I): 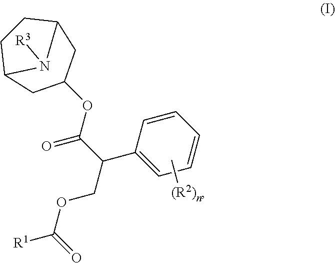
or a pharmaceutically acceptable salt thereof, wherein:
R³ is selected from H, C₁₋₆ alkyl, and C₁₋₆ haloalkyl;
each R² is independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino;
n is selected from 0, 1, 2, 3, 4, and 5; and
R¹ is selected from C₇₋₃₄ alkyl, C₇₋₃₄ alkenyl, C₇₋₃₄ alkynyl, C₃₋₁₀ cycloalkyl, 4-10 membered heterocycloalkyl, 5-14 membered heteroaryl, and C₆₋₁₀ aryl, wherein said C₇₋₃₄ alkyl, C₇₋₃₄ alkenyl, and C₇₋₃₄ alkynyl are each optionally substituted with 1 or 2 substituents independently selected from C₃₋₁₀ cycloalkyl, 4-10 membered heterocycloalkyl, 5-14 membered heteroaryl, and C₆₋₁₀ aryl, and each of said C₃₋₁₀ cycloalkyl, 4-10 membered heterocycloalkyl, 5-14 membered heteroaryl, and C₆-10 aryl is optionally substituted with 1, 2, or 3 substituents independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino.

10. A pharmaceutical composition comprising:
i) a compound of Formula (I): 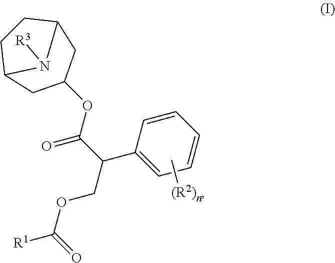
or a pharmaceutically acceptable salt thereof, wherein:
R³ is selected from H, C₁₋₆ alkyl, and C₁₋₆ haloalkyl;
each R² is independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino;
n is selected from 0, 1, 2, 3, 4, and 5; and
R¹ is selected from C₇₋₃₄ alkyl, C₇₋₃₄ alkenyl, C₇₋₃₄ alkynyl, C₃₋₁₀ cycloalkyl, 4-10 membered heterocycloalkyl, 5-14 membered heteroaryl, and C₆₋₁₀ aryl, wherein said C₇₋₃₄ alkyl, C₇₋₃₄ alkenyl, and C₇₋₃₄ alkynyl are each optionally substituted with 1 or 2 substituents independently selected from C₃₋₁₀ cycloalkyl, 4-10 membered heterocycloalkyl, 5-14 membered heteroaryl, and C₆₋₁₀ aryl, and each of said C₃₋₁₀ cycloalkyl, 4-10 membered heterocycloalkyl, 5-14 membered heteroaryl, and C₆-10 aryl is optionally substituted with 1, 2, or 3 substituents independently selected from C₁₋₃ alkyl, C₁₋₃ haloalkyl, C₁₋₃ alkoxy, C₁₋₃ haloalkoxy, OH, NO₂, CN, halo, amino, C₁₋₃ alkylamino, di(C₁₋₃ alkyl)amino, carbamyl, carboxy, aminosulfonyl, and aminosulfonylamino; and
ii) a pharmaceutically acceptable carrier comprising a semifluorinated alkane.

11. A method of treating or preventing a disease or condition where muscarinic acetylcholine receptor is implicated, the method comprising administering to a subject in need thereof a therapeutically effective amount of a pharmaceutical composition of claim 10.

12. A method of treating or preventing a disease or condition where muscarinic acetylcholine receptor is implicated, the method comprising administering to a subject in need thereof a therapeutically effective amount of a pharmaceutical composition of claim 4.
